(19) United States
(12) Patent Application Publication
(10) Pub. No.: US 2011/0038855 A1
(43) Pub. Date: Feb. 17, 2011

(54) TRAIL AND METHODS OF MODULATING T CELL ACTIVITY AND ADAPTIVE IMMUNE RESPONSES USING TRAIL

(75) Inventors: Stephen P. Schoenberger, Encinitas, CA (US); Douglas R. Green, Germantown, TN (US); Edith M. Janssen, San Diego, CA (US); Nathalie M. Droin, Memphis, TN (US)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: LA JOLLA INSTITUTE FOR ALLERGY AND IMMUNOLOGY, La Jolla, CA (US)

(21) Appl. No.: 12/417,487

(22) Filed: Apr. 2, 2009

Related U.S. Application Data
(62) Division of application No. 11/361,307, filed on Feb. 24, 2006, now abandoned.
(60) Provisional application No. 60/656,561, filed on Feb. 24, 2005.

(51) Int. Cl. A61K 39/395; C12N 5/0783; A61P 37/00; A61P 25/00

(52) U.S. Cl. 424/133.1; 435/372.3; 424/173.1; 424/144.1

(57) ABSTRACT

Methods of modulating a T cell response are provided. Methods include, among other things, contacting a T cell that expresses TNF-related apoptosis-inducing ligand (TRAIL, Apo-2L) or TRAIL receptor (DR4 or DR5) with a molecule that binds to TRAIL (Apo-2L), a molecule that binds to TRAIL receptor (DR4 or DR5), or with a soluble TRAIL (Apo-2L) reagent.

### RELATED APPLICATIONS

This application is claims the benefit of priority of U.S. Application Ser. No. 60/656,561, filed Feb. 24, 2005, which is expressly incorporated herein by reference.

### GOVERNMENT SPONSORSHIP

This work was supported in part by a grant from National Institutes of Health (RO1CA81261). The government may have certain rights in the invention.

### TECHNICAL FIELD

The invention relates to modulating TRAIL (Apo-2L) or TRAIL receptor (DR4 or DR5), expression or activity in vitro, ex vivo and in vivo.

### INTRODUCTION

The “help” provided by CD4⁺ T lymphocytes during priming of CD8⁺ T lymphocytes confers a key feature of immune memory: the capacity for autonomous secondary expansion following re-encounter with antigen. Once primed in the presence of CD4+ T cells, helped CD8⁺ T cells acquire the ability to undergo a second round of clonal expansion autonomously upon restimulation in the absence of T help. Helpless CD8⁺ T cells primed in the absence of CD4⁺ T cells, in contrast, can mediate effector functions such as and cytokine secretion upon restimulation but do not undergo a second round of clonal expansion. These disparate responses have features of being “programmed,” i.e. guided by signals transmitted to naïve CD8⁺ T cells during priming which encode specific fates for their clonal progeny.

Control of viral infections such as HIV typically involves synergistic activities of various components of the immune system. In addition to specific antibodies, CD8⁺ T cells are believed to be critical for controlling the infection.

### SUMMARY

Methods of modulating T cell responses in vitro, ex vivo and in vivo are provided. In one embodiment, a method includes contacting a T cell that expresses TNF-related apoptosis-inducing ligand (TRAIL, Apo-2L) or TRAIL receptor (DR4 or DR5) with a molecule that binds to TRAIL (Apo-2L), a molecule that binds to TRAIL receptor (DR4 or DR5), or with a soluble TRAIL (Apo-2L) reagent, for example, a molecule selected from Table 2. Exemplary T cell responses that can be modulated include a memory response, expression or secretion of a chemokine or cytokine, or expression of a receptor that binds to a chemokine or cytokine, cytotoxicity, T cell proliferation, activation-induced cell death (AICD) or apoptosis. An additional exemplary T cell response that can be modulated is AICD or apoptosis due to a secondary exposure of CD8+ T cells to an antigen, where the CD8+ T cells were primed with the antigen in the absence of CD4+ T cell help.

Methods of rescuing T cells (e.g., CD8+ T cells) primed in the absence of CD4+ cell help from apoptosis are also provided. In one embodiment, a method includes contacting T cells (e.g., CD8+ T cells) with an amount of an inhibitor of TRAIL (Apo-2L) or TRAIL receptor (DR4 or DR5) expression or activity sufficient to rescue T cells (e.g., CD8+ T cells) primed in the absence of CD4+ cell help from apoptosis.

Methods of promoting or inducing apoptosis or death of T cells are further provided. In one embodiment, a method includes contacting T cells with an amount of an activator of TRAIL receptor (DR4 or DR5) expression or activity sufficient to promote or induce apoptosis or death of T cells.

Methods of treating a physiological condition, disorder, illness, disease or symptom of a subject that is ameliorated by promoting or inducing T cell apoptosis or death are additionally provided. In one embodiment, a method includes administering an amount of an activator of TRAIL receptor (DR4 or DR5) expression or activity effective to promote or induce T cell apoptosis or death, thereby ameliorating the physiological condition, disorder, illness, disease or symptom. Exemplary conditions, disorders, illness, diseases or symptoms ameliorated by promoting or inducing T cell apoptosis or death include undesirable or aberrant immune responses, undesirable or aberrant inflammatory responses and inflammation, which include, for example, autoimmune disorders and diseases, transplant rejection and graft-versus-host disease.

Methods of inhibiting or preventing activation-induced T cell death (AICD) in a subject having or at risk of having aberrant or undesirable activation-induced T cell death are moreover provided. In one embodiment, a method includes administering to a subject an amount of an inhibitor of TRAIL (Apo-2L) expression or activity sufficient to inhibit or prevent activation-induced T cell death.

Methods of treating a subject that is HIV positive are yet also provided. In one embodiment, a method includes treating a subject that has TRAIL (Apo-2L) producing CD8+ cells, and administering an effective amount of an inhibitor of TRAIL (Apo-2L) expression or activity to the subject to treat HIV, or a physiological condition, disorder, illness, disease or symptom caused by or associated with HIV. Additional exemplary methods embodiments are where CD8+ cells are specific for an antigen (e.g., a bacteria, virus such as HIV, fungi, parasite, prion, cancer or tumor antigen), where antigen specific CD8+ cells are specific for an HIV antigen, where antigen specific CD8+ cells produce TRAIL upon re-encounter of the antigen to which the CD8+ cells were primed. Further exemplary methods embodiments result in an improved or increased immune response against HIV, such as cytotolytic T lymphocyte (CTL) response, following treatment.

Methods of vaccinating a subject are yet further provided. In one embodiment, a method includes administering an inhibitor of TRAIL (Apo-2L) expression or activity prior to, concurrently with or following vaccination of a subject with an antigen. Additional exemplary methods embodiments are where the subject has CD8+ cells specific for an antigen (e.g., a bacteria, virus such as HIV, hepatitis, or herpesvirus, fungi, parasite, prion, cancer or tumor antigen), or where antigen specific CD8+ cells are specific for an HIV antigen, or where antigen specific CD8+ cells produce TRAIL upon re-encounter of the antigen to which the CD8+ cells were primed.

Methods of increasing a cytotolytic T lymphocyte (CTL) response in a subject are yet additionally provided. In one embodiment, a method includes administering an amount of an inhibitor of TRAIL (Apo-2L) expression or activity sufficient to increase the CTL response in the subject. Additional exemplary methods embodiments further include administering an antigen prior to, concurrently with or following administering the inhibitor of TRAIL (Apo-2L) expression or activity to the subject. Further exemplary methods embodiments are where the subject has TRAIL (Apo-2L) producing CD8+ cells, or where the subject has TRAIL (Apo-2L) producing CD8+ cells specific for an antigen.

Identification, screening, monitoring and diagnostic methods are provided. In an identification/screening embodiment, a method of identifying/screening an agent that modulates a T cell response mediated at least in part by TRAIL (Apo-2L), includes: contacting a T cell with a test agent; measuring a T cell response mediated at least in part by TRAIL (Apo-2L) in the presence of the test agent; and determining whether the test agent modulates the T cell response. Modulation of the T cell response identifies the test agent as an agent that modulates a T cell response mediated at least in part by TRAIL (Apo-2L). In another identification/screening embodiment, a method of identifying/screening an agent that inhibits or prevents apoptosis of CD8+ T cells primed in the absence of CD4+ cells, wherein said apoptosis is mediated at least in part by TRAIL (Apo-2L), includes: contacting a CD8+ T cell with a test agent; measuring CD8+ T cell numbers in the presence of the test agent; and determining whether the test agent inhibits or prevents apoptosis of CD8+ T cells. Inhibiting or preventing reduced numbers of CD8+ T cells, or increasing numbers of CD8+ T cells, identifies the test agent as an agent that inhibits or prevents apoptosis of CD8+ T cells. In a further identification/screening embodiment, a method of identifying/screening the presence of non-memory cells, T cells primed without CD4+ T cell help, includes determining TRAIL (Apo-2L) expression or secretion by T cells is TRAIL (Apo-2L). Expression or secretion by T cells identifies the presence of non-memory cells, T cells primed without CD4+ T cell help.

In yet another identification/screening embodiment, a method of identifying/screening non-memory cells, T cells primed without CD4+ T cell help, includes: obtaining cells from a subject; contacting the cells with an antigen; and determining TRAIL (Apo-2L) expression or secretion by the cells. TRAIL (Apo-2L) expression on the cells or soluble TRAIL (Apo-2L) secreted by the cells identifies the non-memory cells. In still another identification/screening embodiment, a method of identifying/screening a subject (e.g., immunosuppressed, is HIV positive, has reduced numbers of Cd4+ T cells, has reduced numbers of antigen-specific CD8+ cells, or is suffering from a progressive reduction in CD4+ cell numbers) that is a candidate for TRAIL (Apo-2L) suppressive therapy, includes: providing a biological sample comprising lymphocytes from a subject; and assaying the sample for CD8+ cells that produce TRAIL (Apo-2L). The presence of CD8+ cells that produce TRAIL (Apo-2L) identifies the subject as a candidate for TRAIL (Apo-2L) suppressive therapy. In still a further identification/screening embodiment, a method of identifying/screening a subject that is a candidate for vaccination or immunization with an antigen, includes: providing a biological sample comprising lymphocytes from a subject; and assaying the sample to determine if CD8+ cells specific for the antigen produce TRAIL (Apo-2L). CD8+ cells specific for the antigen that do not produce TRAIL (Apo-2L) identifies the subject as a candidate for vaccination or immunization with the antigen.

In a monitoring embodiment, a method includes monitoring amounts of non-memory cells, T cells primed without CD4+ T cell help includes determining TRAIL (Apo-2L) expression of CD8+ T cells. The amount of TRAIL (Apo-2L) indicates amounts of non-memory cells, T cells primed without CD4+ T cell help.

In a diagnostic embodiment, a method of diagnosing a physiological disorder or disease associated with undesirable or abnormal high or low amounts of non-memory cells, T cells primed without CD4+ T cell help, includes determining an amount of TRAIL (Apo-2L) produced by CD8+ T cells. The amount of TRAIL (Apo-2L) indicates the presence of or predisposition towards a physiological disorder or disease associated with undesirable or abnormal high or low amounts of non-memory cells, T cells primed without CD4+ T cell help. In another diagnostic embodiment, a method of diagnosing a subject having a deficient immune response against an antigen, includes: providing a biological sample comprising lymphocytes from a subject; and assaying the sample to determine if CD8+ cells specific for the antigen produce TRAIL (Apo-2L). Detecting CD8+ cells specific for the antigen that produce TRAIL (Apo-2L) diagnoses the subject as having a deficient immune response against the antigen.

Exemplary modulators of TRAIL (Apo-2L) or TRAIL receptor (DR4 or DR5) include molecules selected from Table 2. Additional molecules include antibodies and binding fragments thereof, antisense nucleic acid (e.g., RNA, a DNA, triplex forming nucleic acid, RNAi), dominant negative polypeptides, a soluble forms of TRAIL receptor (DR4 or DR5).

Exemplary subjects (e.g., mammals such as humans) include immunocompromised subjects, such as those having or at risk of having relative low or reduced numbers of activity of CD4+ T cells (e.g., ability to provided CD8+ T cells help during priming with an antigen). Exemplary subjects also include those having reduced numbers of antigen-specific CD8+ cells, or is suffering from a progressive reduction or loss of CD4+ cell numbers, or has less than 600/cubic millimeter (mm3) blood CD4+ cells, or less than 300/cubic millimeter (mm3) blood CD4+ cells, or less than 200/cubic millimeter (mm3) blood CD4+ cells, or less than 40% CD4+ cells as a percentage of all lymphocytes in blood, or less than 25% CD4+ cells as a percentage of all lymphocytes in blood, or less than 15% CD4+ cells as a percentage of all lymphocytes in blood. Exemplary subjects further include those having TRAIL (Apo-2L) producing CD8+ cells, for example, TRAIL (Apo-2L) producing CD8+ cells specific for an antigen (e.g., a bacteria, virus such as HIV or hepatitis, fungi, parasite, prion, cancer or tumor antigen), and which antigen specific CD8+ cells produce TRAIL upon re-encounter of the antigen (i.e., a secondary or subsequent exposure to the antigen to which the CD8+ cells were primed). Exemplary subjects additionally include subjects that have been or are a candidate for immunization or vaccination against an antigen, such as a microorganism antigen (infectious agent or pathogen), or a tumor or cancer antigen. Exemplary subjects moreover include subjects that are afflicted with a chronic or acute bacterial, viral (e.g., HIV, hepatitis or herpesvirus), fungal, parasite or prion infection.

### DRAWING DESCRIPTIONS

FIG. 1a-1b. Caspase inhibitors restore secondary expansion in helpless CD8⁺ T cells. (a) CFSE dilution profile of IFN-γ-producing E1B₁₉₂₋₂₀₀-specific CD8⁺ T cells from immunizes intact (helped) and CD4-depleted (helpless) mice were measured directly ex vivo (open histogram) and after stimulation in vitro (shaded histogram). Results are representative histograms from two independent studies (4-5 mice per group). (b) Secondary expansion of IFN-γ-producing E1B₁₉₂₋₂₀₀-specific CD8⁺ T cells from immunized intact (helped) and CD4-depleted (helpless) mice determined by intracellular IFN-γ staining after in vitro restimulation. Expansion of E1B₁₉₂₋₂₀₀-specific helped (filled bars) and helpless CD8⁺ T cells (open bars) was calculated as the fold-increase in the absolute number of specific CD8⁺ T cells. Data are shown as mean±sem (n=4-5 mice/group).

FIG. 2a-2e. TRAIL expression prevents secondary expansion of helpless CD8⁺ T cells. (a) mRNA levels for the apoptosis-related genes Bcl-2, Bcl-xL, FasL, TNF-α; TRAIL, DR5, and c-FLIP were determined by real-time RT-PCR at indicated time points following in vitro E1B₁₉₂₋₂₀₀-peptide restimulation of purified CD8⁺ T cells obtained from immunized intact (filled circle) and CD4-depleted mice (open circle). (b) The frequency of IFN-γ-producing E1B-specific CD8⁺ T cells of immunized intact (filled circle) and CD4-depleted mice (open circle) was determined directly ex vivo and again following coculture with Tap^{+/+}Ad5E1-MEC. Fold expansion of IFN-γ producing E1B₁₉₂₋₂₀₀-specific CD8⁺ T cells was calculated as the increase in the absolute number of specific CD8⁺ T cells. (c) Immunized intact (black bars) and CD4-depleted mice (white bars) were re-challenged with act-mOVA/K^{b-/-}-splenocytes and the absolute number of OVA₂₅₇₋₂₆₄-specific CD8⁺ T cells per spleen was determined by intracellular IFN-γ staining. (d) Purified CD8⁺ T cells from Tap^{-/-}Ad5E1-MEC immunized intact (black bars) and CD4-depleted mice (white bars) were cultured with Tap^{+/+}Ad5E1-MEC in the presence or absence of the indicated soluble death receptors. Fold expansion of IFN-γ producing E1B₁₉₂₋₂₀₀-specific CD8⁺ T cells was calculated as the increase in the absolute number of specific CD8+ T cells. (e) Purified CD8⁺ T cells from Tap^{-/-}Ad5E1-MEC-immunized intact (filled bars) and CD4-depleted mice (open bars) were cotransfected with empty pGLOW or pGLOW containing the bp −397 of human TRAIL promoter in presence of the β-galactosidase-expressing vector. Data are shown as mean±sem (n=4-5 mice/group).

FIG. 3a-3c. TRAIL-mediated defective secondary expansion of LCMV-specific helpless CD8⁺ T cells. (a) mRNA levels for Bcl-2, Bcl-xL, FasL, TNF, TRAIL, DR5, and c-FLIP were determined by real-time RT-PCR at indicated time points following in vitro GP₃₃₋₄₁-peptide restimulation of purified CD8⁺ T cells obtained from immunized intact (filled circle) and CD4-depleted mice (open circle). (b) Purified CD8⁺ T cells from immunized intact (filled bars) and CD4-depleted mice (open bars) were cultured with LCMV-infected thioglycollate-induced macrophages in the presence or absence of soluble death receptors. Fold expansion was calculated as the increase in the absolute number of GP₃₃₋₄₁-specific CD8⁺ T cells. (c) Purified CD8⁺ T cells from intact (filled bars) and CD4-depleted mice infected 28 days earlier (open bars) were cotransfected with empty pGLOW or pGLOW containing the bp −397 of human TRAIL promoter in presence of the β-galactosidase-expressing vector. GFP mRNA was analyzed. Data are shown as mean±sem (n=5-6 mice/group).

FIG. 4a-4d. Suicide and fratricide by helpless CD8⁺ T cell-derived TRAIL. Purified CD8+ T cells from (a) intact wild type mice and (b) intact and CD4-depleted TRAIL^{-/-} mice (helped, filled bar; helpless, open bar) were cultured and the fold expansion of E1B₁₉₂₋₂₀₀-specific CD8⁺ T cells was determined. (c) Purified helped (filled bar) and helpless (open bar) CD8⁺ T cells were cultured and the fold expansion of E1B₁₉₂₋₂₀₀-specific CD8⁺ T cells was determined. (d) helped CD8⁺ T cells were cultured and the fold expansion of the helped CD8⁺ T cells was determined as the increase in the absolute number of E1B₁₉₂₋₂₀₀-specific CD8⁺ T cells. Data are shown as mean±sem (n=3 mice/group).

FIG. 5. Primary responses of helped and helpless E1B₁₉₂₋₂₀₀-specific CD8⁺ T cells in mice genetically modified for various apoptosis related genes. Immunized intact (filled circle) and CD4-depleted mice (open circle) from the indicated strains were purified and the frequency of E1B₁₉₂₋₂₀₀-specific CD8⁺ T cells was determined by intracellular IFN-γ stain. The frequency of effector CD8⁺ T cells generated in the absence of CD4 T cells is generally 40-60% of that produced in wildtype (intact) animals, as previously reported (Ehst et al., Am J Transplant 3:1355 (2003)). Data are shown as mean±sem (n=4-5 mice/group).

FIG. 6. Analysis of secondary expansion in vitro by OVA₂₅₇₋₂₆₄/K^{b} tetramers. Immunized intact (black bar) and CD4-depleted mice (white bar) from the indicated strains were stimulated with irradiated MEC.B7.Sig-OVA cells ant the expansion of OVA₂₅₇₋₂₆₄-specific CD8⁺ T cells was calculated as the fold-increase in the absolute number of specific CD8⁺ T cells. Data are shown as mean±sem (n=4 mice/group).

FIG. 7. TRAIL expression in LCMV-specific helpless CD8⁺ T cells. Immunized intact (black bar) and CD4-depleted mice (white bar) were purified by flow cytometry and the mRNA level for TRAIL was determined by real-time RT-PCR. Data are shown as mean±sem (n=2 mice/group). Purity of sorted tetramer-positive population was 97%.

FIG. 8a-8d. HIV peptides induce release of TRAIL in PBMC of HIV-infected donors with low CD4⁺ T cell counts. PBMC of 12 HIV-infected subjects with CD4⁺ T cell counts below 200 CD4⁺ T cells/mm³ (HIV⁺CD4^{low}) and 8 donors with CD4⁺ T cell counts above 200 CD4 cells/mm³ (HIV⁺CD4^{high}) as well as 22 non-HIV-infected healthy donors (HIV⁻CD4^{high}) were analyzed in a TRAIL (a) and (b) and IFN-γ (c) and (d) ELISPOT assay in the presence of an HIV peptide library (a) and (c) or of positive (CEF) or negative (HCV) control peptides (b) and (d) as specified by the symbols. Solid circles show the responses to HIV peptides in (a) and (c), open circles to CEF peptides in (b) and (d). Solid squares represent the responses to HCV peptides in (b) and (d). The cumulative number of TRAIL or IFN-γ spots induced by the individual peptides within the specific peptide library is represented by the symbols for each donor.

FIG. 9a-9b. HIV-peptide-induced TRAIL-secreting cells are CD8 positive. (a) CD8 T cell-depletion. Bulk PBMC (solid bars) and CD8 T cell-depleted PBMC (open bars, <0.01% residual CD8⁺ T cells) were analyzed in a TRAIL ELISPOT assay for reactivity to the specified peptides, as described previously. Results are shown for an individual donor showing the mean and SE of spot numbers induced by the peptide analyzed in duplicate wells. The data are representative for 121 individual HIV peptides inducing TRAIL production in six donors. (b) CD8⁺ T cell-enrichment. Bulk PBMC (solid bars) and purified CD8⁺ T cells as obtained by negative selection (hatched bars, >97% purity) were analyzed in a TRAIL ELISPOT assay, as previously described. Results are depicted for an individual donor showing the mean and SE of spot numbers induced by the specified peptides analyzed in duplicate wells. The data are representative for 72 individual HIV peptides inducing TRAIL-production in four donors.

FIG. 10a-10f. Representative ELISPOT wells showing HIV-peptide-induced production of TRAIL or IFN-γ by CD8⁺ T cells. Purified CD8⁺ T cells of HIV-peptide-induced ELISPOTs are shown on the right with the corresponding medium control on the left. (a) to (d) TRAIL and IFN-γ spots for purified CD8⁺ T cells as specified. (e) Spot size distribution histogram for TRAIL and IFN-γ spots produced by PBMC in HIV⁺CD4^{low} donors, as specified. Cumulative data obtained by ImmunoSpot analysis of 2500 spots in each category are shown. (f) Spontaneous TRAIL release in HIV⁺CD4^{low} (n=21), HIV⁺CD4^{high} (n=8) and HIV⁻CD4^{high} (n=22) control donors. Each data point represents the mean of duplicate wells for a single donor with SE<20%.

FIG. 11. Dissociated production of TRAIL and IFN-γ by HIV peptide-specific CD8⁺ T cells. PBMC from an individual HIV⁺CD4^{low} donors were analyzed for specific HIV-peptide-induced production of TRAIL (open bars) and IFN-γ (solid bars). ELISPOT assays for TRAIL and IFN-γ were performed in parallel. The mean of duplicate wells is represented for each peptide and TRAIL/IFN-γ combination (SE<20%). The data are representative for 34 individual HIV peptides analyzed in this donor, and for 938 peptides analyzed in PBMC of 21 CD4^{low} donors.

### DETAILED DESCRIPTION

The invention provides, among other things, compositions and methods embodiments for modulating a T cell response, in vitro, ex vivo or in vivo. In one embodiment, a method includes contacting a T cell that expresses TNF-related apoptosis-inducing ligand (TRAIL, also referred to as Apo-2L and TNFSF10, since TRAIL is a member of TNF super family 10) or TRAIL receptor (DR4 or DR5) with a molecule that binds to TRAIL (Apo-2L), a molecule that binds to TRAIL receptor (DR4 or DR5), or with a soluble TRAIL (Apo-2L) reagent.

The term “bind,” or “binding,” when used in reference to a molecule that binds to TRAIL (Apo-2L) or TRAIL receptor (DR4 or DR5), means that the molecule directly or indirectly contacts TRAIL (Apo-2L) or TRAIL receptor (DR4 or DR5), at least transiently. To bind or binding therefore means direct physical contact, or indirect, by binding to an intermediary. An example of an intermediary is a molecule that binds to TRAIL (Apo-2L) or TRAIL receptor (DR4 or DR5). Thus, a molecule that binds to TRAIL (Apo-2L) or TRAIL receptor (DR4 or DR5) may or may not physically contact TRAIL (Apo-2L) or TRAIL receptor (DR4 or DR5), but may bind to an intermediary molecule that, in turn, contacts TRAIL (Apo-2L) or TRAIL receptor (DR4 or DR5).

Binding molecules (e.g., agents and compounds) include molecules that can modulate an activity or expression of TRAIL (Apo-2L) or TRAIL receptor (DR4 or DR5). The term “modulate” means any change in activity or expression, for example, to increase, stimulate, induce, enhance or promote activity or expression, or to decrease, reduce, inhibit, delay, halt, eliminate or prevent activity or expression. Binding molecules therefore include agents that can increase, stimulate, induce, enhance or promote an activity or expression of TRAIL (Apo-2L) or TRAIL receptor (DR4 or DR5), and agents that can decrease, reduce, inhibit, delay, halt, eliminate or prevent an activity or expression of TRAIL (Apo-2L) or TRAIL receptor (DR4 or DR5). Binding molecules that bind to or interact with TRAIL (Apo-2L) or TRAIL receptor (DR4 or DR5) and increase, stimulate, induce, enhance or promote an activity or expression of TRAIL (Apo-2L) or TRAIL receptor (DR4 or DR5), can be referred to as TRAIL (Apo-2L) or TRAIL receptor (DR4 or DR5) agonists. Binding molecules that decrease, reduce, inhibit, delay, halt, eliminate or prevent an activity or expression of TRAIL (Apo-2L) or TRAIL receptor (DR4 or DR5) can be referred to as TRAIL (Apo-2L) or TRAIL receptor (DR4 or DR5) antagonists.

Exemplary non-limiting examples of molecules that bind to TRAIL (Apo-2L) or TRAIL receptor (DR4 or DR5), including inhibitors (e.g., antagonists) and activators (e.g., agonists) of TRAIL (Apo-2L) and TRAIL receptor (DR4 or DR5) expression or activity, are set forth in Table 2. Additional examples include, for example, molecules that bind to TRAIL (Apo-2L) or TRAIL receptor (DR4 or DR5), such as soluble TRAIL (Apo-2L) reagent (e.g., soluble form of TRAIL receptor (DR4 or DR5)), or an antibody (e.g., polyclonal or monoclonal human, humanized, primatized or chimeric), that binds to TRAIL (Apo-2L), such as N2B1 and antibody N2B2 (Kayagaki et al., J. Immuonol. 163:1906 (1999)) exemplified herein; or an antibody that binds to TRAIL receptor (DR4 or DR5). Further non-limiting examples include, a TRAIL (Apo-2L) or a TRAIL receptor (DR4 or DR5) antisense nucleic acid, a dominant negative TRAIL (Apo-2L) or TRAIL receptor (DR4 or DR5) polypeptide, or a soluble TRAIL receptor (DR4 or DR5).

Methods embodiments include methods of contact or administration, in vitro (in solution in solid phase or in culture), ex vivo and in vivo, with molecules that bind to TRAIL (Apo-2L) or TRAIL receptor (DR4 or DR5), including inhibitors (e.g., antagonists) and activators (e.g., agonists) of TRAIL (Apo-2L) or TRAIL receptor (DR4 or DR5) expression or activity. Such methods modulate, among other things, T cells or a T cell response, for example. T cell responses that can be modulated in accordance with various embodiments include, for example, a memory response, T cell cytotoxicity, T cell proliferation, activation-induced cell death (AICD) or apoptosis, cytokine or chemokine expression or secretion, or cytokine or chemokine receptor expression or secretion.

The term “contact” means direct or indirect binding or interaction between two or more entities (e.g., between TRAIL (Apo-2L) or TRAIL receptor (DR4 or DR5), and a molecule (e.g., an antibody) that binds to TRAIL (Apo-2L) or TRAIL receptor (DR4 or DR5). Contacting as used herein includes in solution, in solid phase, in culture, in vitro, ex vivo, in a cell and in vivo. Contacting in vivo can be referred to as administering, or administration.

The invention also provides, among other things, compositions and methods embodiments for decreasing, reducing, inhibiting, preventing or rescuing T cells primed in the absence of sufficient CD4+ cell help from apoptosis or cell death (e.g., CD8+ T cells). In one embodiment, a method includes contacting T cells (e.g., CD8+ cells, CD8+ T cells primed in the absence of CD4+ T cell help, antigen-specific CD8+ T cells that produce TRAIL upon antigen re-encounter) with an amount of an inhibitor of TRAIL (Apo-2L) or TRAIL receptor (DR4 or DR5) activity or expression sufficient to rescue T cells (e.g., CD8+ T cells, CD8+ T cells primed in the absence of CD4+ T cell help, antigen-specific CD8+ T cells that produce TRAIL upon antigen re-encounter) primed in the absence of CD4+ cells from apoptosis.

The invention further provides, among other things, compositions and methods embodiments for increasing, stimulating, inducing, enhancing or promoting apoptosis or death of T cells (e.g., CD4+, CD8+, activation-induced CD8+ T cell death, CD8+ T cells primed in the absence of CD4+ T cell help, antigen-specific CD8+ T cells that produce TRAIL upon antigen re-encounter). In one embodiment, a method includes contacting T cells with an amount of an activator of TRAIL receptor (DR4 or DR5) expression or activity sufficient to promote or induce apoptosis or death of T cells.

The invention additionally provides, among other things, compositions and methods embodiments for decreasing, reducing, inhibiting, or preventing apoptosis or death of T cells (e.g., CD4+, CD8+, activation-induced CD8+ T cell death, CD8+ T cells primed in the absence of CD4+ T cell help, antigen-specific CD8+ T cells that produce TRAIL upon antigen re-encounter). In one embodiment, a method includes contacting T cells in a subject at risk of undesirable activation-induced T cell death with an amount of an inhibitor of TRAIL (Apo-2L) activity or expression sufficient to inhibit or prevent activation-induced T cell death. In one aspect, activation-induced T cell death is caused by increased apoptosis or cell death following a secondary antigen exposure of CD8+ T cells initially primed with the antigen in the absence of sufficient CD4+ T cell help.

The invention moreover provides, among other things, compositions and methods embodiments for treating a physiological condition, disorder, illness, disease or symptom that is ameliorated by increasing, stimulating, inducing, enhancing or promoting T cell apoptosis or death (e.g., CD4+, CD8+, activation-induced CD8+ T cell death CD8+ T cells primed in the absence of CD4+ T cell help, antigen-specific CD8+ T cells that produce TRAIL upon antigen re-encounter). In one embodiment, a method includes administering an amount of an activator of TRAIL receptor (DR4 or DR5) expression or activity to a subject effective to promote or induce T cell apoptosis or death, thereby ameliorating the physiological condition, disorder, illness, disease or symptom.

The invention still further provides, among other things, compositions and methods embodiments for treating a subject that is HIV positive, wherein the subject has TRAIL (Apo-2L) producing CD8+ T cells (e.g., antigen-specific CD8+ T cells). In one embodiment, a method includes administering an effective amount of an inhibitor of TRAIL (Apo-2L) expression or activity to the subject to treat HIV, or a physiological condition, disorder, illness, disease or symptom caused by or associated with HIV.

The invention still additionally provides, among other things, compositions and methods embodiments for immunizing or vaccinating a subject with an antigen (e.g., a subject that has TRAIL (Apo-2L) producing CD8+ T cells that are specific against the antigen or etiological agent that comprises the antigen). In various embodiments, a molecule that binds to and modulates TRAIL (Apo-2L) or TRAIL receptor (DR4 or DR5) expression or activity, such as an inhibitor (e.g., antagonist), or an activator (e.g., agonist) is administered prior to, concurrently with, or following immunization or vaccination with an antigen (e.g., bacterial, viral such as HIV or hepatitis, fungal, parasite, tumor or cancer, or prion antigen). In one embodiment, a method includes administering an inhibitor of TRAIL (Apo-2L) expression or activity prior to, concurrently with or following immunization or vaccination of a subject with an antigen.

The invention moreover provides, among other things, compositions and methods embodiments for increasing a cytotolytic T lymphocyte (CTL) response in a subject. In one embodiment, a method includes administering an amount of an inhibitor of TRAIL (Apo-2L) expression or activity sufficient to increase the CTL response in the subject. In another embodiment, a method includes administering an amount of an inhibitor of TRAIL (Apo-2L) expression or activity and an antigen (e.g., bacterial, viral such as HIV or hepatitis, fungal, parasite, prion, or tumor or cancer antigen) sufficient to increase the CTL response in the subject. In various aspects, the antigen is administered prior to, concurrently with or following administering the inhibitor of TRAIL (Apo-2L) expression or activity to the subject.

In various compositions and methods embodiments, the treated subject is immunosuppressed (e.g., is HIV positive, has an immunosuppressive condition, disorder, disease or illness, or is undergoing, has undergone or is a candidate for immunosuppressive treatment or therapy), has reduced numbers of CD4+ cells, has reduced numbers of antigen-specific CD8+ cells (e.g., that produce TRAIL (Apo-2L) upon re-encounter with the antigen), or is suffering from a progressive reduction or loss in CD4+ cell numbers, or has less than 600/cubic millimeter (mm3) blood CD4+ cells, or less than 300/cubic millimeter (mm3) blood CD4+ cells, or less than 200/cubic millimeter (mm3) blood CD4+ cells, or has less than 40% CD4+ cells as a percentage of all lymphocytes in blood, or less than 25% CD4+ cells as a percentage of all lymphocytes in blood, or less than 15% CD4+ cells as a percentage of all lymphocytes in blood. In further compositions and methods embodiments, the treated subject has antigen specific CD8+ T cells that produce TRAIL (Apo-2L), for example, a bacterial, viral, fungal, parasite, prion, or tumor or cancer antigen.

The term “treatment” refers to contact or administration to a subject that has a physiological condition, disorder, illness or disease (e.g., an HIV infection), or exhibits one or more symptoms or pathologies associated with or caused by the physiological condition, disorder, illness or disease (e.g., HIV infection). The term “therapeutic,” when used in reference to treatment, means that the treatment is practiced on a subject that has a physiological condition, disorder, illness or disease, or exhibits one or more symptoms or pathologies associated with or caused by the physiological condition, disorder, illness or disease (e.g., HIV infection). Treatment methods are intended to provide an objective or subjective (perceived) effect or benefit, e.g., an improvement in a physiological condition, disorder, illness, disease or symptom of a subject, but due to variability in responsiveness, not all subjects will respond to treatment.

“Prophylaxis” and grammatical variations thereof refer to contact, administration or in vivo delivery to a subject prior to a known physiological condition, disorder, illness, disease or symptom thereof (e.g., an HIV infection). In situations where it is not known if a subject has the physiological condition, disorder, illness, disease, or symptom, contact with, administration or ex vivo or in vivo delivery of a molecule that binds to TRAIL (Apo-2L), or a molecule that binds to TRAIL receptor (DR4 or DR5) to a subject occurs prior to manifestation or onset of a symptom of the physiological condition, disorder, illness or disease. In such a method, the effect of contact with, administration, ex vivo or in vivo delivery of a molecule that binds to TRAIL (Apo-2L), TRAIL receptor (DR4 or DR5) can be to eliminate, prevent, inhibit, decrease or reduce the probability of or susceptibility towards developing a physiological condition, disorder, illness, disease or a symptom thereof.

Methods embodiments include treating physiological conditions, disorders, illnesses, diseases and symptoms treatable by administering or contact ex vivo or in vivo with a molecule that binds to TRAIL (Apo-2L), or a molecule that binds to TRAIL receptor (DR4 or DR5). Treating physiological conditions, disorders, illnesses, diseases and symptoms ameliorated by administering or contact with a molecule that binds to TRAIL (Apo-2L), or a molecule that binds to TRAIL receptor (DR4 or DR5) are also included.

Non-limiting examples include physiological conditions, disorders, illnesses, diseases and symptoms caused by or associated with undesirable numbers or activated CD8+ T cells (naïve or memory) or insufficient or reduced numbers or activity (e.g., interferon production) of CD8+ T cells (naïve or antigen-primed, such as in the absence of CD4+ T cells) that contributes to, stimulates, enhances or mediates the physiological condition, disorder, illnesses, disease or symptom. Non-limiting examples also include physiological conditions, disorders, illnesses, diseases and symptoms caused by undesirable numbers or activated CD4+ T cells, or insufficient or reduced numbers or activity of CD4+ T cells that contributes to, stimulates, enhances or mediates the physiological condition, disorder, illness, disease or symptom. Non-limiting examples further include autoimmune disorders or disease, transplant rejection and graft-versus-host disease. Non-limiting examples additionally include multiple sclerosis, autoimmune diabetes, autoimmune hepatitis, primary biliary cirrhosis, myelodysplastic syndrome, aplastic anemia and polymyostitis. Non-limiting examples moreover include physiological conditions, disorders, illnesses, diseases and symptoms resulting in immunosuppression, such as acute or chronic infections (bacterial, viral such as HIV, fungal, parasite, prion) immunosuppressive therapy or treatment, cancers and tumors.

As used herein, the term “associated with,” when used in reference to the relationship between a physiological condition, disorder, illness, disease, or symptom, and an effect or consequence of the physiological condition, disorder, illness, disease, symptom, means that the effect or consequence is caused by the condition, disorder or disease, or is a secondary effect or consequence of the physiological condition, disorder, illness, disease, or symptom. A symptom that is present in a subject may therefore be a direct result of or caused by the condition, or may be an indirect result of the physiological condition, disorder, illness, disease, or symptom. For example, certain physiological conditions, disorders, illnesses, diseases, and symptoms that occur during HIV are due in part to weakness of the immune system of the infected subject.

Methods of treatment embodiments extend to one or more symptoms, pathologies, or side effects of a physiological condition, disorder, illness, disease, symptom or an effect or consequence of the physiological condition, disorder, illness, disease or symptom. A symptom, pathology or side effect that is present in a subject may be the direct result of or caused by the physiological condition, disorder, illness or disease, or may be due at least in part to a secondary or subsequent effect, such as the subject reacting or responding to (e.g., an immunological response) the physiological condition, disorder, illness or disease. Such secondary effects are considered to be associated with the condition, disorder, illness, disease or symptom.

Methods embodiments, including, for example, treatment methods, are applicable to treating any physiological condition, disorder, illness, disease and symptom treatable by administering or contact with a molecule that binds to TRAIL (Apo-2L), or a molecule that binds to TRAIL (Apo-2L) receptor (DR4 or DR5). Although not wishing to be bound by any particular theory, in immunocompromised (immunosuppressed) subjects with relatively reduced numbers of CD4+ T cells, naïve CD8+ T cells primed with antigen in the absence of CD4+ T cell help are rendered “helpless.” Upon re-encounter of the antigen, the antigen-specific CD8+ T cells typically do not undergo clonal expansion, exhibit reduced cytokine secretion, produce TRAIL (Apo-2L), and undergo apoptosis (AICD) or death, leading to an impaired immune response against the antigen (e.g., an impaired CTL response), and a weakened ability to control the infection (bacterial, viral, fungal, parasite, prion, etc.) or disease (e.g., tumors or cancers). The methods embodiments therefore include treatment of immunocompromised (immunosuppressed) subjects generally, as well as subjects having particular physiological conditions, disorders, illnesses, diseases and symptoms, or therapies that can cause or result in transient or longer term immunosuppression.

Although not wishing to be bound by any particular theory, in immunosuppressed subjects, such as HIV positive (+) subjects with a relative reduction in CD4+ T cell numbers, CD8+ T cells primed with antigen (e.g., HIV antigen) in the absence of CD4+ T cell help produce TRAIL (Apo-2L) upon re-encounter of the antigen (e.g., HIV antigen), which can lead to apoptosis of antigen-specific CD8+ T cell and other (nearby) cells (e.g., other lymphocytes, such as CD4+ T cells) thereby impairing the subject's immune response against the antigen-bearing agent (e.g., HIV), other opportunistic infections (bacterial, viral, fungal, parasite, prion, etc.), disorders or diseases (e.g., tumors or cancers). CD8+ T cells primed with other antigens (e.g., from exposure to an opportunistic infection or disease, or vaccination or immunization) in the absence of CD4+ T cell help, upon re-exposure to the antigen, may produce TRAIL (Apo-2L) and become committed to activation induced cell death (AICD). This reduction or loss of antigen-specific CD8+ T cells may lead to ineffective responses against opportunistic infections, disorders and diseases, as well as ineffective immunization or vaccination in subjects with reduced CD4+ T cell numbers. The methods embodiments therefore include treatment of immunosuppressed/immunocompromised subjects generally, and any physiological condition, disorder, illness, disease, symptom, and therapy or treatment that causes or results in reduction or loss of CD4+ T cells, such as immunosuppressive therapies and treatments, chronic infections such as HIV, hepatitis and herpesvirus, immunosuppressive-associated opportunistic infections (bacterial, viral, fungal, parasite, prion, etc.), disorders and diseases (e.g., tumors or cancers), and vaccination or immunization protocols.

HIV includes any strain or isolate or subtype or species of HIV, or combination of strains or isolates or subtypes or species of HIV. Particular examples are HIV-1 and HIV-2. Specific non-limiting examples of HIV-1 groups include Groups M, N and O. Additional examples are drug resistant HIV types, groups, subtypes or isolates. Specific non-limiting examples of HIV-1 subtypes include A, B, A/B, A/E, A/G, C, D, F, G, H, J and K subtypes, and mixtures thereof.

Methods embodiments can result in a beneficial effect or improvement in a subjects' physiological condition, disorder, illness, disease or symptom. An example of a beneficial effect or improvement is an objective or subjective reduction, decrease, inhibition, delay, ameliorating or prevention of onset, progression, severity, duration, frequency or probability of one or more symptoms or pathologies associated with or caused by a.

Methods embodiments therefore include, among other things, treatment methods that result in a beneficial effect. A beneficial effect includes modulating numbers or activity (e.g., a T cell response) of lymphocytes (e.g., CD4, CD8+ T cells, CD8+ T cells primed in the absence of CD4+ T cell help, antigen-specific CD8+ T cells that produce TRAIL upon antigen re-encounter). For example, increasing or stabilizing or preventing or inhibiting a reduction or loss of lymphocyte (e.g., CD4+ or CD8+ T cells) numbers or activity (e.g., a T cell response) can be used to treat immunocompromised (immunosuppressed) subjects (HIV + subjects, immunosuppressed subjects, subjects undergoing an immunosuppressive therapy or treatment, subjects with a tumor or cancer, etc.). Decreasing, stabilizing, or preventing or inhibiting increases in lymphocyte numbers or activity can be used to treat undesirable or aberrant immune responses, conditions, disorders, or diseases such as autoimmunity.

Lymphocytes include CD8+ (naïve and CD8+ T cells primed in the absence of CD4+ T cell help, antigen-specific CD8+ T cells that produce TRAIL upon antigen re-encounter) and CD4+ T cells. Typical numbers of CD8+ T cells range from about 150-1,000 cells/cubic millimeter (mm3) blood in normal (non-immunosuppressed subjects). Typical numbers of CD4+ T cells range from about 500-1,500 cells/cubic millimeter (mm3) blood in normal (non-immunosuppressed subjects). Typical numbers of CD4+ T cells, expressed as a percentage of total lymphocytes, range from about 30-70%. In a normal (non-immunosuppressed) subject T-cell ratio, CD4+/CD8+, is usually between 0.9 and 6.0.

Methods embodiments include increasing or stabilizing, or preventing or inhibiting a reduction or loss of, lymphocyte numbers or activity (e.g., CD8+, naïve or CD8+ T cells primed in the absence of CD4+ T cell help, antigen-specific CD8+ T cells that produce TRAIL upon antigen re-encounter, or CD4+ T cells and responses thereof) in a subject. In one embodiment, a method includes administering a sufficient amount of an inhibitor (e.g., antagonist) of TRAIL (Apo-2L) or TRAIL receptor (DR4 or DR5) expression or activity to increase or stabilize, or prevent or inhibit a reduction of, numbers or activity (e.g., a T cell response) of CD8+ (naïve or CD8+ T cells primed in the absence of CD4+ T cell help, antigen-specific CD8+ T cells that produce TRAIL upon antigen re-encounter) or CD4+ T cells in the subject (e.g., an immunosuppressed or HIV+ subject). In various aspects, CD8+ or CD4+ T cell counts less than 600, 500, 400, 300, or 200 cells/cubic millimeter (mm3) blood are increased or stabilized, CD8+ or CD4+ T cell counts less than 200 cells/cubic millimeter (mm3) blood are increased or stabilized, or the percentage of CD8+ or CD4+ T cells less than 60%, 50%, 40%, 25%, or 15% of all lymphocytes is increased or stabilized in the subject (e.g., an immunosuppressed or HIV+subject). In further aspects, CD8+ (naïve or CD8+ T cells primed in the absence of CD4+ T cell help, antigen-specific CD8+ T cells that produce TRAIL upon antigen re-encounter) or CD4+ T cell activity (e.g., a memory response, T cell cytotoxicity, T cell proliferation, cytokine or chemokine or receptor expression or secretion) is increased or stabilized in the subject (e.g., an immunosuppressed or HIV+ subject).

Methods embodiments further include decreasing or stabilizing or preventing or inhibiting increases of, lymphocyte numbers or activity (e.g., CD8+ naïve or CD8+ T cells primed in the absence of CD4+ T cell help, antigen-specific CD8+ T cells that produce TRAIL upon antigen re-encounter) in a subject. In one embodiment, a method includes administering a sufficient amount of an activator (e.g., agonist) of TRAIL (Apo-2L) or TRAIL receptor (DR4 or DR5) expression or activity (e.g., a T cell response) to decrease or stabilize, or prevent or inhibit increases of, numbers of CD8+ (naïve or CD8+ T cells primed in the absence of CD4+ T cell help, antigen-specific CD8+ T cells that produce TRAIL upon antigen re-encounter) or CD4+ T cells in the subject (e.g., with an undesirable or aberrant immune response, condition, disorder, illness, or disease such as autoimmunity). In various aspects, CD8+ (naïve or CD8+ T cells primed in the absence of CD4+ T cell help, antigen-specific CD8+ T cells that produce TRAIL upon antigen re-encounter) or CD4+ T cell activity (e.g., a memory response, T cell cytotoxicity, T cell proliferation, cytokine or chemokine or receptor expression or secretion) is decreased or stabilized in the subject (e.g., with an undesirable or aberrant immune response, condition, disorder, or disease such as autoimmunity).

Methods embodiments of treatment therefore include providing a beneficial effect to a subject, for example, reducing, decreasing, inhibiting, delaying, ameliorating or preventing onset, progression, severity, duration, frequency or probability of HIV infection or pathogenesis or one or more symptoms or pathologies associated with or caused by HIV infection or pathogenesis; reducing, decreasing, inhibiting, delaying or preventing increases in HIV titer, viral load, replication, proliferation, or an amount of a viral protein of one or more HIV strains or isolates or subtypes. Stabilizing the infection, a symptom or pathology thereof, or preventing, inhibiting or delaying a worsening or progression of the infection or a symptom or pathology associated with or caused by HIV infection or pathogenesis, or progression of the underlying HIV infection, are also included in various embodiments of the treatment methods.

Specific non-limiting examples of symptoms and pathologies associated with or caused by HIV infection or pathogenesis (e.g., illness), whose onset, progression, severity, frequency, duration or probability can be reduced, decreased, inhibited, delayed ameliorated or prevented include, for example, fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, an opportunistic disorder, nerve damage, encephalopathy, dementia and death.

Specific non-limiting examples of symptoms and pathologies associated with or caused by HIV infection or pathogenesis (e.g., illness), whose onset, progression, severity, frequency, duration or probability can be reduced, decreased, inhibited, delayed ameliorated or prevented also include, for example, opportunistic disorders (e.g., bacterial, viral, fungal, and parasitic infections), cancers and tumors. Non-limiting examples of opportunistic disorders include Candidiasis of bronchi, trachea, lungs or esophagus, cervical cancer, Coccidioidomycosis, Cryptococcosis, Cryptosporidiosis, Bacillary Angiomatosis, Cytomegalovirus (CMV), Cytomegalovirus retinitis, Herpes virus, Hepatitis virus, papilloma virus, Histoplasmosis, Isosporiasis, Kaposi's sarcoma, Burkitt's lymphoma, immunoblastic lymphoma, Mycobacterium avium, Mycobacterium tuberculosis, Pneumocystis carinii, Pneumonia, progressive multifocal leukoencephalopathy (PML), Salmonelosis, Toxoplasmosis, Wasting syndrome and Lymphoid interstitial pneumonia/pulmonary lymphoid type. Other symptoms and pathologies of HIV infection or pathogenesis (e.g., illness), are known in the art and treatment thereof in accordance with the methods is provided.

For HIV, a beneficial effect or improvement therefore includes providing a subject with partial or complete protection against infection or pathogenesis (e.g., illness), or a symptom caused by an HIV infection or pathogenesis. Particular non-limiting examples of beneficial effects also include reducing, decreasing, inhibiting, delaying or preventing HIV infection or pathogenesis, and reducing, decreasing, inhibiting, ameliorating or preventing onset, severity, duration, progression, frequency or probability of one or more symptoms or pathologies associated with an HIV infection or pathogenesis. Additional non-limiting examples of beneficial effects include reducing, decreasing, amounts of, or inhibiting, delaying or preventing increases in HIV titer or load, proliferation or replication. Further non-limiting particular examples of beneficial effects include reducing, decreasing, inhibiting, delaying, ameliorating or preventing onset, progression, severity, duration, frequency, probability or susceptibility of a subject to an HIV infection or pathogenesis (e.g., illness), or accelerating, facilitating or hastening recovery of a subject from an HIV infection or pathogenesis or one or more associated symptoms, pathologies or adverse side effects.

Specific non-limiting improvements for HIV can therefore be, for example, in any of fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, opportunistic infections disorders and diseases, nerve damage, encephalopathy, dementia, death, CD8+ (naïve or CD8+ T cells primed in the absence of CD4+ T cell help, antigen-specific CD8+ T cells that produce TRAIL upon antigen re-encounter) or CD4+ T cell numbers, activity or percentages of CD8+ (naïve or CD8+ T cells primed in the absence of CD4+ T cell help, antigen-specific CD8+ T cells that produce TRAIL upon antigen re-encounter) or CD4+ T cell numbers relative to all lymphocytes, an improved or increased cytotolytic T lymphocyte (CTL) response against HIV or opportunistic disorders, etc., to any degree or for any duration of time (hours, days, weeks, months, years, or cure).

An additional example of a beneficial effect or improvement includes increasing or stabilizing, or inhibiting reductions of, numbers or activity of CD8+ (naïve or CD8+ T cells primed in the absence of CD4+ T cell help, antigen-specific CD8+ T cells that produce TRAIL upon antigen re-encounter) or CD4+ T cells, (e.g., greater than 600, 500, 400, 300 or 200 cells/cubic millimeter (mm3) blood). A further symptom that may be treated includes increasing the percentage of CD8+ or CD4+ T cells relative to other lymphocytes, or stabilizing the percentage of CD8+ or CD4+ T cells relative to other lymphocytes (e.g., greater than 40%, 25%, or 15%).

Additional non-limiting examples of a beneficial effect or improvement include reducing or decreasing probability, susceptibility or likelihood that the subject so treated will manifest one or more symptoms of the physiological condition, disorder, illness, or disease. Symptoms and physiological or psychological responses caused by or associated with the various physiological conditions, disorders, illnesses, and diseases are set forth herein and are known in the art and, therefore, improvements in these and other adverse symptoms or physiological or psychological responses are included in the various treatment embodiments.

Methods embodiments also include treatment of a cell-proliferative or hyperproliferative condition, disorder, illness, or disease. Non-limiting examples include metastatic or non-metastatic tumors, cancers, malignancies and neoplasia of any cell, organ or tissue origin. The terms “tumor,” “cancer,” “malignancy,” and “neoplasia” are used interchangeably and refer to a cell or population of cells whose growth, proliferation or survival is greater than growth, proliferation or survival of a normal counterpart cell, e.g. a cell proliferative or differentiative disorder. Such disorders can affect virtually any cell or tissue type, e.g., carcinoma, sarcoma, melanoma, neural, and reticuloendothelial or haematopoietic neoplastic disorders (e.g., myeloma, lymphoma or leukemia). Tumors and cancers arise from a multitude of tissues and organs, including but not limited to breast, lung, thyroid, head and neck, brain, lymphoid, gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, which may or may not metastasize to other secondary sites.

The tumor or cancer may be in any stage, e.g., early or advanced, such as a stage I, II, III, IV or V tumor. The tumor or cancer may have been subject to a prior treatment, be stabilized (non-progressing) or in remission.

Cells comprising a tumor or cancer may be aggregated in a cell mass or be dispersed. A “solid tumor” refers to neoplasia or metastasis that typically aggregates together and forms a mass. Specific non-limiting examples include visceral tumors such as melanomas, breast, pancreatic, uterine and ovarian cancers, testicular cancer, including seminomas, gastric or colon cancer, hepatomas, adrenal, renal and bladder carcinomas, sarcomas, lung, head and neck cancers and brain tumors/cancers.

Melanoma, which refers to malignant tumors of melanocytes and other cells derived from pigment cell origin may arise in the skin, the eye (including retina), or other regions of the body, and may include the cells derived from the neural crest that also gives rise to the melanocyte lineage. A pre-malignant form of melanoma, known as dysplastic nevus or dysplastic nevus syndrome, is associated with melanoma development.

Carcinomas, which refer to malignancies of epithelial or endocrine tissue, include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the uterine cervix, lung, prostate, breast, head and neck, colon, pancreas, testes, adrenal, kidney, esophagus, stomach, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma and oligodendrocytoma

A “liquid tumor,” refers to neoplasia that is diffuse in nature as they do not typically form a solid mass. Particular examples include neoplasia of the reticuloendothelial or haematopoetic system, such as lymphomas, myelomas and leukemias. Non-limiting examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML). Lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Particular examples of a beneficial effect or improvement for a cell-proliferative or hyperproliferative condition, disorder, illness, or disease include a reduction in cell volume (e.g., tumor or cancer size or cell mass), inhibiting an increase in cell volume, a slowing or inhibition of hyperproliferative disorder worsening or progression, stimulating cell lysis or apoptosis, reducing or inhibiting tumor metastasis, reduced mortality, prolonging lifespan. Adverse symptoms and complications associated with a hyperproliferative disorder (e.g., tumor, neoplasia, and cancer) that can be reduced or decreased include, for example, pain, nausea, lack of appetite, weakness and lethargy. Thus, inhibiting or delaying an increase in tumor cell mass or metastasis (stabilization of a disease) can increase lifespan (reduce mortality) even if only for a few days, weeks or months, even though complete ablation of the tumor has not resulted. A reduction in the occurrence, frequency, severity, progression, or duration of the underlying condition, disorder, illness, or disease, or a symptom of the condition, disorder, illness, or disease, such as an improvement in subjective feeling (e.g., increased energy, appetite, reduced nausea, improved mobility or psychological well being, etc.), are all examples of beneficial effect or improvement.

Particular non-limiting examples of a beneficial effect or improvement for an infection (e.g., an infectious agent such as a bacteria, virus, fungi, parasite, prion, etc.) include reducing or decreasing occurrence, frequency, severity, progression, or duration of one or more symptoms or complications of infection. Additional particular non-limiting examples of a beneficial effect or improvement for an infection include reducing, inhibiting, decreasing or preventing increases in infectious agent titer, infectious agent replication, infectious agent proliferation, or an infectious agent protein or nucleic acid sequence. Further particular non-limiting examples of beneficial effect or improvement for an infectious agent include stabilizing the condition (i.e., preventing or inhibiting a worsening or progression of a symptom or complication associated with infection, or progression of the infection). Symptoms or complications associated with infection whose occurrence, frequency, severity, progression, or duration can be reduced, decreased or prevented are known in the art. A beneficial effect or improvement can also include hastening or accelerating recovery from pathogen infection.

For a bacterial, viral, fungal, parasite or prion infection or pathogenesis, a beneficial effect or improvement in titer, bacterial or viral load, replication, bacterial, viral, fungal, parasite or prion proliferation, or an amount of a bacterial, viral, fungal, parasite or prion protein can be a result of treatment. A beneficial effect or improvement would also include reducing, inhibiting or preventing increases in titer, bacterial or viral load, replication, bacterial, viral, fungal, parasite or prion proliferation, or an amount of a bacterial, viral, fungal, parasite or prion protein of one or more bacterial, viral, fungal, parasite or prion strains or isolates or subtypes or species (e.g., HIV-1, HIV-2, etc.). A beneficial effect or improvement would further include stabilizing a symptom or pathology associated with or caused by the infection or pathogenesis, or inhibiting, decreasing, delaying or preventing a worsening or progression of the symptom or pathology associated with or caused by the infection or pathogenesis, or a progression of the underlying infection.

Method embodiments include treatment of physiological conditions, disorders, illnesses, diseases or symptoms, caused by or associated with undesirable and aberrant immune responses, immune disorders and immune diseases. In various embodiments, methods include treating chronic and acute forms of undesirable or aberrant inflammatory responses and inflammation; treating chronic and acute forms of undesirable or aberrant proliferation, survival, differentiation, death, or activity of a lymphocyte, such as a T cell (e.g., CD8+ T cell, CD8+ T cells primed in the absence of CD4+ T cell help, antigen-specific CD8+ T cells that produce TRAIL upon antigen re-encounter). Methods include contact or administering a molecule that binds to TRAIL (Apo-2L) or TRAIL receptor (DR4 or DR5).

As used herein, an “undesirable immune response” or “aberrant immune response” refers to any immune response, activity or function that is greater or less than desired or physiologically normal. An undesirable immune response, function or activity can be a normal response, function or activity. Thus, normal immune responses so long as they are undesirable, even if not considered aberrant, are included within the meaning of these terms. An undesirable immune response, function or activity can also be an abnormal response, function or activity. An abnormal (aberrant) immune response, function or activity deviates from normal. Undesirable and aberrant immune responses can be humoral, cell-mediated or a combination thereof, either chronic or acute.

A non-limiting example of an undesirable or aberrant immune response is where the immune response is hyper-responsive, such as in the case of an autoimmune condition, disorder, illness or disease. Another example of an undesirable or aberrant immune response is where an immune response leads to acute or chronic inflammatory response or inflammation systemically, regionally or locally, in any tissue or organ. Yet another example of an undesirable or aberrant immune response is where an immune response leads to destruction of cells, tissue or organ, such as a transplant, as in graft vs. host disease. Still another example of an undesirable or aberrant immune response is where the immune response is hypo-responsive, such as where response to an antigen is less than desired. For example, a reduced CTL response to a pathogen (e.g., HIV) can result in increased susceptibility or a more severe infection. Tolerance to a tumor-associated antigen (TAA) is thought to contribute to the ability of tumors to evade immune surveillance thereby surviving and proliferating in afflicted subjects.

The terms “immune disorder” and “immune disease” mean an immune function or activity that is greater than (e.g., autoimmunity) or less than (e.g., immunodeficiency) desired. Immune disorders and diseases can be characterized by different physiological symptoms or abnormalities, depending upon the disorder or disease.

Particular non-limiting examples of immune disorders and diseases to which the methods embodiments apply include autoimmune disorders and immunodeficiencies. Methods embodiments for treating autoimmune and immunosuppressed conditions, disorders, illnesses, diseases or symptoms are therefore provided.

Autoimmune disorders are generally characterized as an undesirable or aberrant increased or inappropriate response, activity or function of the immune system. Immunodeficiencies are generally characterized by decreased or insufficient humoral or cell-mediated immune responsiveness or memory, or undesirable tolerance.

Exemplary autoimmune disorders treatable in various methods embodiments include rheumatoid arthritis (RA), juvenile rheumatoid arthritis, osteoarthritis, psoriatic arthritis, diabetes mellitus, multiple sclerosis (MS), encephalomyelitis, myasthenia gravis, systemic lupus erythematosus (SLE), autoimmune thyroiditis, atopic dermatitis, eczematous dermatitis, psoriasis, Sjögren's Syndrome, Crohn's disease, inflammatory bowel disease (IBD), aphthous ulcer, iritis, conjunctivitis, keratoconjunctivitis, ulcerative colitis, asthma, allergic asthma, cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus (IDDM, type I diabetes), insulin-resistant diabetes mellitus (type II diabetes), immune-mediated infertility, autoimmune Addison's disease, pemphigus vulgaris, pemphigus foliaceus, dermatitis herpetiformis, autoimmune alopecia, Vitiligo, autoimmune hemolytic anemia, autoimmune thrombocytopenic purpura, pernicious anemia, Guillain-Barre syndrome, Stiff-man syndrome, acute rheumatic fever, sympathetic ophthalmia, Goodpasture's syndrome, systemic necrotizing vasculitis, antiphospholipid syndrome and allergies (e.g., allergic asthma).

Exemplary immunodeficiency treatable in accordance with the invention include severe combined immunodeficiency (SCID) such as recombinase activating gene (RAG 1/2) deficiency, adenosine deaminase (ADA) deficiency, interleukin receptor γ chain (γ_{C}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency; predominantly antibody deficiencies such as X-linked agammaglobulinemia (Bruton's tyrosine kinase deficiency); autosomal recessive agammaglobulinemia such as Mu heavy chain deficiency; surrogate light chain (γ5/14.1) deficiency; Hyper-IgM syndrome either X-linked (CD40 ligand deficiency) and others; Ig heavy chain gene deletion; IgA deficiency; deficiency of IgG subclasses (with or without IgA deficiency); common variable immunodeficiency (CVID); antibody deficiency with normal immunoglobulins; transient hypogammaglobulinemia of infancy; interferon γ receptor (IFNGR1, IFNGR2) deficiency; interleukin 12 and interleukin 12 receptor deficiency; immunodeficiency with thymoma; Wiskott-Aldrich syndrome (WAS protein deficiency); ataxia telangiectasia (ATM deficiency); X-linked lymphoproliferative syndrome (SH2D1A/SAP deficiency); and hyper IgE syndrome). Exemplary immunodeficiencies also include disorders associated with or secondary to another disease (e.g., chromosomal instability or defective repair such as Bloom syndrome, Xeroderma pigmentosum, Fanconi anemia, ICF syndrome, Nijmegen breakage syndrome and Seckel syndrome; chromosomal defects such as Down syndrome (Trisomy 21), Turner syndrome and Deletions or rings of chromosome 18 (18p- and 18q-); skeletal abnormalities such as short-limbed skeletal dysplasia (short-limbed dwarfism) and cartilage-hair hypoplasia (metaphyseal chondroplasia); immunodeficiency associated with generalized growth retardation such as Schimke immuno-osseous dysplasia, Dubowitz syndrome, Kyphomelic dysplasia with SCID, Mulibrey's nannism, Growth retardation, facial anomalies and immunodeficiency and Progeria (Hutchinson-Gilford syndrome); immunodeficiency with dermatologic defects such as ectrodactyly-ectodermal dysplasia-clefting syndrome, immunodeficiency with absent thumbs, anosmia and ichthyosis, partial albinism, Dyskeratosis congenita, Netherton syndrome, Anhidrotic ectodermal dysplasia, Papillon-Lefevre syndrome and congenital ichthyosis; hereditary metabolic defects such as acrodermatitis enteropathica, transcobalamin 2 deficiency, type 1 hereditary orotic aciduria, intractable diarrhea, abnormal facies, trichorrhexis and immunodeficiency, methylmalonic acidemia, biotin dependent carboxylase deficiency, mannosidosis, glycogen storage disease, type 1b, Chediak-Higashi syndrome; hypercatabolism of immunoglobulin such as familial hypercatabolism, intestinal lymphangiectasia; chronic muco-cutaneous candidiasis; hereditary or congenital hyposplenia or asplenia; and Ivermark syndrome.

Additional examples of immune conditions, disorders, illnesses, diseases and symptoms to which the methods embodiment apply include inflammatory responses and inflammation. Inflammatory responses and inflammation are generally characterized as an undesirable or aberrant increased or inappropriate inflammatory response, or an activity or function of the immune system that causes or is associated with inflammation.

Exemplary inflammatory responses and inflammation treatable in accordance with the invention include inflammatory responses and inflammation caused by or associated with proliferation, survival, differentiation, death, or activity of T cells, antigen presenting cells (e.g., dendritic cells) or B cells. Methods (e.g., treatment) include reducing, inhibiting or preventing occurrence, progression, severity, frequency or duration of a symptom or characteristic of an inflammatory response or inflammation. At the whole body, regional or local level, an inflammatory response or inflammation is generally characterized by swelling, pain, headache, fever, nausea, skeletal joint stiffness or lack of mobility, rash, redness or other discoloration, or tissue or cell damage. At the cellular level, an inflammatory response or inflammation is characterized by one or more of cell infiltration of the region, production of antibodies (e.g., autoantibodies), production of cytokines, lymphokines, chemokines, interferons and interleukins, cell growth and maturation factors (e.g., differentiation factors), cell proliferation, cell differentiation, cell accumulation or migration and cell, tissue or organ damage. Methods embodiments include treatment at the whole body, regional or local level, as well as at the cellular level.

Undesirable or aberrant inflammation or an inflammatory response, mediated by cellular or humoral immunity, may cause, directly or indirectly, cell, tissue or organ damage, either to multiple cells, tissues or organs, or specifically to a single cell type, tissue type or organ. Exemplary tissues and organs that can exhibit damage include epidermal or mucosal tissue, gut, bowel, pancreas, thymus, liver, kidney, spleen, skin, or a skeletal joint (e.g., knee, ankle, hip, shoulder, wrist, finger, toe, or elbow). Treatment can result in reducing, inhibiting or preventing progression or worsening of tissue damage. For example, treatment can result in reducing, inhibiting or preventing damage to a transplanted cell, tissue or organ (e.g., graft vs. host disease). Such treatment methods can be performed prior to, concurrently with, immediately following or after transplant of a cell, tissue or organ in a subject.

As used herein, the terms “transplant,” “transplantation” and grammatical variations thereof mean grafting, implanting, or transplanting a cell, tissue or organ from one part of the body to another part, or from one individual or animal to another individual or animal. The transplanted cell, tissue or organ may therefore be an allograft or xenograft. Exemplary transplant cells include neural cells. Exemplary transplant tissues include skin, blood vessel, eye and bone marrow. Exemplary transplant organs include heart, lung, liver and kidney. The term also includes genetically modified cells, tissue and organs, e.g., by ex vivo gene therapy in which the transformed cells, tissue and organs are obtained or derived from a subject (e.g., human or animal) and then reintroduced into the same or a different subject (e.g., human or animal).

Treatment embodiments also include reducing or eliminating a need, dosage amount or frequency of another treatment, such as another drug or other agent used for treatment. For example, a subject having or at risk of having HIV infection or pathogenesis or a symptom or pathology associated with or caused by HIV infection or pathogenesis may no longer require or may require less of another treatment for HIV infection or pathogenesis, a symptom or pathology associated with or caused by HIV, or an adverse side effect caused by HIV.

Non-limiting exemplary HIV treatments that may be eliminated or used at reduced doses or frequencies of administration include protease inhibitors, reverse transcriptase inhibitors, virus fusion inhibitors and virus entry inhibitors. Additional non-limiting exemplary HIV treatments that may be eliminated or reduced are as set forth herein or known in the art.

A treatment that provides a beneficial effect or improvement need not be complete ablation of any particular physiological condition, disorder, illness, disease, or symptom caused by or associated with the physiological condition, disorder, illness, disease, or symptom. Rather, that provides a beneficial effect or improvement may be any objective or subjective measurable or detectable effect, benefit or improvement in the physiological condition, disorder, illness, disease, or symptom, of a treated subject. A detectable beneficial effect or improvement includes a subjective or objective reduction in the occurrence, frequency, severity, progression, or duration of a physiological condition, disorder, illness, disease, or symptom thereof, including the underlying cause or a consequence of the physiological condition, disorder, illness, disease, or symptom thereof, or a reversal of the physiological condition, disorder, illness, disease, or symptom thereof. A treatments that provides a beneficial effect or improvement, “ameliorate” is used synonymously, therefore need not be complete ablation of any or all adverse symptoms or complications associated with the physiological condition, disorder, illness, disease, or symptom, but is any measurable or detectable, objectively or subjectively, effect, benefit or improvement in the physiological condition, disorder, illness, disease, or symptom thereof. Thus, reducing, inhibiting, decreasing, eliminating, delaying, halting or preventing a progression or worsening of the physiological condition, disorder, illness, disease, or symptom is a satisfactory outcome.

For example, a molecule that binds to and modulates TRAIL (Apo-2L) or TRAIL receptor (DR4 or DR5) expression or activity, such as an inhibitor (e.g., antagonist), may stabilize or increase or inhibit a reduction of CD8+ (naïve or CD8+ T cells primed in the absence of CD4+ T cell help, antigen-specific CD8+ T cells that produce TRAIL upon antigen re-encounter) or CD4+ T cell numbers or activity, or percentages of CD8+ (naïve or CD8+ T cells primed in the absence of CD4+ T cell help, antigen-specific CD8+ T cells that produce TRAIL upon antigen re-encounter) or CD4+ T cell numbers relative to all lymphocytes, to any degree or for any duration of time, but not affect fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, opportunistic infections, disorders and diseases, nerve damage, encephalopathy, dementia or ultimately death. Another example is where a molecule that binds to and modulates TRAIL (Apo-2L) or TRAIL receptor (DR4 or DR5) activity or expression, such as an inhibitor (e.g., antagonist), reduces or inhibits one symptom, e.g., fatigue or headache, without detectably improving one or more other symptoms. Thus, a treatment is achieved when there is an incremental improvement in the subject's condition or a partial reduction or a stabilization of a physiological condition, disorder, illness, disease, or adverse symptom (stabilizing one or more symptoms or pathologies), over a short or long duration (hours, days, weeks, months, years, or cure).

In embodiments of the methods in which there is a desired outcome, for example, a treatment method that provides an objective or subjective beneficial effect or improvement in a physiological condition, disorder, illness, disease, or symptom, or adverse side effect, a composition, e.g., a molecule that binds to TRAIL (Apo-2L) or TRAIL receptor (DR4 or DR5), can be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable, measurable or desirable (subjective or objective) outcome, such as a detectable improvement or measurable beneficial or desirable effect for a given subject, of any degree or for any time period or duration (e.g., for minutes, hours, days, months, years, or cured).

A “sufficient amount” or “effective amount” therefore includes decreasing, reducing, inhibiting, preventing, or delaying onset; decreasing, reducing, inhibiting, delaying, or preventing a progression or worsening of a physiological condition, disorder, illness, disease, or adverse symptom; or reducing, relieving, ameliorating, or alleviating, severity, frequency, duration, susceptibility or probability of a physiological condition, disorder, illness, disease, or symptom. In addition, hastening a subject's recovery from a physiological condition, disorder, illness, disease, or symptom is considered a sufficient or effective amount. Various beneficial effects and indicia of therapeutic and prophylactic benefit are as set forth herein and are known to the skilled artisan.

Amounts, frequencies or duration also considered sufficient and effective are those that result in the elimination or a reduction in amount, frequency or duration of another compound, agent, treatment or therapeutic regimen. For example, a treatment method is considered as having a beneficial or therapeutic effect if contact, administration or delivery in vivo results in the use of a lesser amount, frequency or duration of another compound, agent, treatment or therapeutic regimen to treat the physiological condition, disorder, illness, disease, or symptom.

A sufficient amount or an effective amount can but need not be provided in a single administration and can but need not be administered alone (i.e., without a second drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, since additional doses, amounts, frequency or duration of administration above and beyond such doses, or additional compounds, agents, treatments or therapeutic regimens may be included in order to be effective or sufficient in a given subject.

A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or less response to embodiments of the methods than other subjects.

Any compound, agent, treatment or other therapeutic regimen having a desired, beneficial, additive, synergistic or complementary activity or effect can be formulated or used in a combination with or in addition to embodiments of the methods. Methods embodiments therefore include additional treatments, protocols and therapies, which include any other composition, treatment, protocol or therapeutic regimen. In various aspects, the compound, agent, treatment or therapeutic regimen is for providing a subject with protection against, treatment of, decreasing susceptibility towards, treating an associated disorder (e.g., opportunistic infection or disease) caused by or associated with the physiological condition, disorder, illness, disease, or symptom.

Thus, in methods embodiments in vitro, ex vivo and in vivo, compositions and methods include a molecule that binds to TRAIL (Apo-2L) or TRAIL receptor (DR4 or DR5), combinations of molecules that bind to TRAIL (Apo-2L) or TRAIL receptor (DR4 or DR5), a molecule that binds to TRAIL (Apo-2L) or TRAIL receptor (DR4 or DR5) combined with other agents or treatments as set forth herein. In various embodiments, compositions and methods include a molecule that binds to TRAIL (Apo-2L) or TRAIL receptor (DR4 or DR5), combinations of molecules that bind to TRAIL (Apo-2L) or TRAIL receptor (DR4 or DR5), a molecule that binds to TRAIL (Apo-2L) or TRAIL receptor (DR4 or DR5) combined with other agents or treatments, for modulating a T cell response; rescuing T cells (e.g., CD8+ T cells) primed in the absence of CD4+ cell help from apoptosis; promoting or inducing apoptosis or death of T cells; inhibiting or preventing apoptosis or death of T cells (e.g., activation-induced CD8+ T cell death); treating physiological conditions, disorders, illness, disease, or symptoms ameliorated by rescuing T cells (e.g., CD8+ T cells) primed in the absence of CD4+ cell help from apoptosis (e.g., immunosuppressed or HIV+), or ameliorated by promoting or inducing T cell apoptosis or death (e.g., autoimmunity); increasing or inducing a cytolytic T lymphocyte (CTL) response (e.g., immunosuppressed or HIV+); decreasing or inhibiting a cytolytic T lymphocyte (CTL) response (e.g., autoimmunity).

For HIV, combination methods embodiments include, for example, anti-HIV drugs, such as protease inhibitors, reverse transcriptase inhibitors, virus fusion inhibitors and virus entry inhibitors, HIV antibodies, etc.), and include contact with, administration in vitro or in vivo, with another compound (e.g., another molecule that binds to TRAIL (Apo-2L) or TRAIL receptor (DR4 or DR5)), agent, treatment or therapeutic regimen appropriate for the condition, disorder, illness, disease or symptom to be treated. The compound (e.g., another molecule that binds to TRAIL (Apo-2L) or TRAIL receptor (DR4 or DR5)), agent, treatment or therapeutic regimen may be used in accordance with any method embodiment, as set forth herein, prior to, concurrently or following contacting or administering a molecule that binds to TRAIL (Apo-2L) or TRAIL receptor (DR4 or DR5) in vitro, ex vivo or in vivo.

Non-limiting examples of combination embodiments include protease inhibitors, reverse transcriptase inhibitors, virus fusion inhibitors and virus entry inhibitors, live or attenuated HIV, HIV proteins and antibodies that bind to HIV proteins. A pool of protease inhibitors, reverse transcriptase inhibitors, virus fusion inhibitors and virus entry inhibitors, live or attenuated HIV, HIV proteins or HIV binding antibodies (e.g., monoclonal or polyclonal) can be combined with a molecule that binds to TRAIL (Apo-2L) or TRAIL receptor (DR4 or DR5) or administered separately (prior to, concurrently with or following) administration of a molecule that binds to TRAIL (Apo-2L) or TRAIL receptor (DR4 or DR5). Specific non-limiting examples of combination embodiments include treatments such as AK602, AMD070, APV, ATV, ATZ, AVX754, AZT, Abacavir, Acyclovir, Adefovir dipivoxil, Adriamycin, Agenerase, Aldesleukin, Alovudine, AmBisome, Amdoxovir, Amphocin, Amphotec, Amphotericin B, Ampligen, Amprenavir, Androderm, Androgel, Aptivus, Atazanavir, Azithromycin, BMS-488043, Bactrim, Baraclude, Biaxin, BufferGel, C31G, CD4-IgG2, CPV, CS, Calanolide A, Capravirine, Carbopol 974P, Carrageenan, Carraguard, Cellulose sulfate, Clarithromycin, Combivir, Copegus, Cotrimoxazole, Crixivan, Cyanovirin-N, Cytovene, DAPD, DLV, DPC 817, DS, Delavirdine, Depo-Testosterone, Dextran sulfate, Didanosine, Diflucan, Doxil, Doxorubicin, Dronabinol, EFV, Efavirenz, Elvucitabine, Emtricitabine, Emtriva, Enfuvirtide, Entecavir, Epivir, Epoetin alfa, Epogen, Epzicom, Etopophos (phosphate salt), Etoposide, Etravirine, Fluconazole, Fortovase, Fosamprenavir, Fungizone, Fuzeon, GSK-873,140 (aplaviroc), GW433908, Gammar-P, Ganciclovir, Growth hormone, Human growth hormone, HEC, Hepsera, Hivid, Hydroxyethyl cellulose, IDV, IGIV, Interleukin-2 (IL-2), INH, Immune Globulin, Indinavir, Interferon alfa-2, Intron A (2b), Invirase, Isoniazid, Itraconazole, KP-1461, Kaletra, L-000870810, LPV/RTV, Lamivudine, Lexiva, Marinol, Megace, Megestrol, Mycobutin, NFV, NVP, Naphthalene 2-sulfonate polymer, Nebupent, Nelfinavir, Neutrexin, Nevirapine, New-Fill, Norvir, Nydrazid, Onxol, PA-457, PMPA, PRO 2000, PRO542, Paclitaxel, Paxene, Pegasys (2a), Pentamidine, Peptide T, Poly(I)-Poly(C12U), Poly-L-lactic acid, Polygam S/D, Procrit, Proleukin, RCV, RTV, RVT, Racivir, Rebetol, Rescriptor, Retrovir, Reverset, Reyataz, Ribavirin, Rifabutin, Rifadin, Rifampin, Rimactane, Ritonavir, Roferon-A (2a), SCH-C, SCH-D (vicriviroc), SQV, Saquinavir, Savvy, Sculptra, Septra, Serostim, Somatropin, Sporanox, Stavudine, Sulfamethoxazole, Sustanon, Sustiva, T-20, TDF, THC, TMC114, TMC125, TNX-355, Taxol, Tenofovir, Tenofovir disoproxil fumarate, Testosterone, Tipranavir, Toposar, Trimethoprim, Trimetrexate, Trizivir, Truvada, UC-781, UK-427,857 (maraviroc), Ushercell, Valcyte, Valganciclovir, Valproic acid, VePesid, Vicriviroc, Videx, Viracept, Viramune, Virazole, Viread, Vitrasert, ZDV, Zalcitabine, Zerit, Ziagen, Zidovudine, Zithromax, Zovirax, D4T, ddC, β-LFddC, P-LFd4C, DDI, f-APV, 3TC, and human erythropoietin (EPO). Still additional non-limiting exemplary treatments include cytokines, chemokines, interferons and interleukins.

Further additional exemplary treatments include HIV protein (e.g., present on one or more of HIV-1 or HIV-2, such as envelope protein gp160, gp120 or gp41, gag protein, pol protein, p7, p17, p24, tat, rev, nef, vif, vpr, vpu, reverse transcriptase, integrase, or protease), an antibody that binds to an HIV protein (e.g., present on one or more of HIV-1 or HIV-2, such as envelope protein gp160, gp120 or gp41, gag protein, pol protein, p7, p17, p24, tat, rev, nef, vif, vpr, vpu, reverse transcriptase, integrase, or protease). HIV proteins and binding antibodies include those present on or that bind to one or more of HIV-1 (e.g., Groups M, N and O, or subtypes include A, B, A/B, A/E, A/G, C, D, F, G, H, J and K subtypes, and mixtures thereof) or HIV-2, drug resistant HIV types, groups, subtypes or isolates.

Still additional examples of combination embodiments include immune system enhancing and anti-cell proliferative treatments (e.g., for treatment of tumors or cancers). Specific non-limiting examples include cytokines, chemokines, interferons and interleukins; cytokine, chemokine, interferon, and interleukin receptors; internal or external radiotherapy (e.g., ionizing or non-ionizing radiation); surgical resection; hyperthermia; and chemotherapeutic drugs and agents.

For a cell proliferative or hyperproliferative condition, disorder, illness, or disease, embodiments include anti-proliferative, anti-tumor, anti-cancer, anti-neoplastic treatments or therapies can inhibit, decrease, retard, slow, reduce or prevent a cell proliferative or hyperproliferative condition, disorder, illness, or disease, such as tumor, cancer or neoplastic growth, progression, metastasis, proliferation or survival, in vitro or in vivo. Particular non-limiting examples of an anti-proliferative (e.g., tumor) therapy include chemotherapy, immunotherapy, radiotherapy (ionizing or chemical), local thermal (hyperthermia) therapy and surgical resection. Any composition, treatment, protocol, therapy or regimen having an anti-cell proliferative activity or effect can be used in combination with a composition or method embodiment.

Anti-proliferative or anti-tumor compositions, therapies, protocols or treatments can operate by biological mechanisms that prevent, disrupt, interrupt, inhibit or delay cell cycle progression or cell proliferation; stimulate or enhance apoptosis or cell death, inhibit nucleic acid or protein synthesis or metabolism, inhibit cell division, or decrease, reduce or inhibit cell survival, or production or utilization of a necessary cell survival factor, growth factor or signaling pathway (extracellular or intracellular). Non-limiting examples of chemical agent classes having anti-cell proliferative and anti-tumor activities include alkylating agents, anti-metabolites, plant extracts, plant alkaloids, nitrosoureas, hormones, nucleoside and nucleotide analogues. Specific examples of drugs having anti-cell proliferative and anti-tumor activities include cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds such as decitabine (5-aza-2′ deoxycytidine), cytarabine, 1-beta-D-arabinofuranosyl-5-azacytosine and dihydro-5-azacytidine, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol.

Additional agents can be employed, for example, monoclonal antibodies that bind tumor cells or oncogene products, such as Rituxan® and Herceptin (Trastuzumab)(anti-Her-2 neu antibody), Bevacizumab (Avastin), Zevalin, Bexxar, Oncolym, 17-1A(Edrecolomab), 3F8 (anti-neuroblastoma antibody), MDX-CTLA4, Campath®, Mylotarg, IMC-C225 (Cetuximab), aurinstatin conjugates of cBR96 and cAC10 (Doronina et al., Nat Biotechnol 21:778 (2003)).

Yet additional examples of combination embodiments include anti-microbial agents and treatments (e.g., for treatment of bacteria, virus, fungi, parasites or prions). Specific non-limiting examples are as set forth herein and are also known in the art.

Anti-inflammatory agents useful in methods embodiments include cytokines and chemokines. Particular non-limiting examples of cytokines include anti-inflammatory cytokines such as IL-4 and IL-10. Anti-cytokines and anti-chemokines, such as antibodies that bind to pro-inflammatory cytokines, TNFα, IFNγ, IL-1, IL-2, IL-5, IL-6, IL-9, IL-13, IL-16, growth factors such as granulocyte/macrophage colony-stimulating factor can be employed, etc. Additional non-limiting examples of agents useful for treating inflammation include antibodies, such as anti-IgE (e.g., rhuMAb-E25 omalizumab), -IgA and -IgG antibodies, receptors and receptor ligands.

Additional non-limiting examples of agents or treatments to include in methods embodiments include immunosuppressive agents such as corticosteroids (steroid receptor agonists) such as budesonide, prednisone, flunisolide, flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers.

A “peptide,” “polypeptide” or “protein” refers to two or more amino acids covalently linked by an amide bond or non-amide equivalent. Polypeptides include full length native polypeptide, and “modified” forms such as subsequences, variant sequences, fusion/chimeric sequences and dominant-negative sequences. Specific non-limiting examples of polypeptides include forms of TRAIL (Apo-2L) or TRAIL receptor (DR4 or DR5)) having antagonistic and agonistic activity on TRAIL (Apo-2L) or TRAIL receptor (DR4 or DR5)) expression or activity. Exemplary human and mouse TRAIL (Apo-2L) polypeptides, respectively, are as follows:

(Human, SEQ ID NO: 1)
MAMMEVQGGP SLGQTCVLIV IFTVLLQSLC VAVTYVYFTN
ELKQMQDKYS KSGIACFLKE DDSYWDPNDE ESMNSPCWQV
KWQLRQLVRK MILRTSEETI STVQEKQQNI SPLVRERGPQ
RVAAHITGTR GRSNTLSSPN SKNEKALGRK INSWESSRSG
HSFLSNLHLR NGELVIHEKG FYYIYSQTYF RFQEEIKENT
KNDKQMVQYI YKYTSYPDPI LLMKSARNSC WSKDAEYGLY
SIYQGGIFEL KENDRIFVSV TNEHLIDMDH EASFFGAFLV
G
(Mouse, SEQ ID NO: 2)
MPSSGALKDL SFSQHFRMMV ICIVLLQVLL QAVSVAVTYM
YFTNEMKQLQ DNYSKIGLAC FSKTDEDFWD STDGEILNRP
CLQVKRQLYQ LIEEVTLRTF QDTISTVPEK QLSTPPLPRG
GRPQKVAAHI TGITRRSNSA LIPISKDGKT LGQKIESWES
SRKGHSFLNH VLFRNGELVI EQEGLYYIYS QTYFRFQEAE
DASKMVSKDK VRTKQLVQYI YKYTSYPDPI VLMKSARNSC
WSRDAEYGLY SIYQGGLFEL KKNDRIFVSV TNEHLMDLDQ
EASFFGAFLI N

Subsequences and fragments refer to polypeptides having one or more fewer amino acids in comparison to a reference (e.g., native) polypeptide sequence. A variant peptide can have a sequence with 50%, 60%, 70%, 75%, 80%, 85%, 90%, 95%, 96%, 97%, 98%, or more identity to a reference sequence. Variant sequences include naturally occurring alterations of sequence, due to intra-species polymorphisms or different species, as well as artificially produced alterations of sequence. Sequence homology between species is in the range of about 70-80%.

Peptides include L- and D-isomers, and combinations thereof. Peptides can include modifications typically associated with post-translational processing of proteins, for example, cyclization (e.g., disulfide or amide bond), phosphorylation, glycosylation, carboxylation, ubiquitination, myristylation, or lipidation. Modified peptides can have one or more amino acid residues substituted with another residue, added to the sequence or deleted from the sequence. Specific examples include one or more amino acid substitutions, additions or deletions (e.g., 1-3, 3-5, 5-10, 10-20, or more).

Peptides synthesized and expressed as fusion proteins have one or more additional domains linked thereto, and are also referred to as chimeric polypeptides. The additional domain(s) may confer an additional function upon the sequence. For example, TRAIL (Apo-2L)-IgG (Fc) or TRAIL receptor (DR4 or DR5))-IgG (Fc) fusion proteins can have antagonistic activity or agonist activity.

The term “fusion,” when used in reference to two or more molecules (e.g., polypeptides) means that the molecules are covalently attached. A particular example for attachment of two protein sequences is an amide bond or equivalent. The term “chimeric,” and grammatical variations thereof, when used in reference to a protein, means that the protein is comprised of one or more heterologous amino acid residues from one or more different proteins.

The term “heterologous,” when used in reference to a polypeptide, means that the polypeptide is not normally contiguous with the other polypeptide in its natural environment. Thus, a chimeric polypeptide means that a portion of the polypeptide does not exist fused with the other polypeptide in normal cells. In other words, a chimeric polypeptide is a molecule that does not normally exist in nature, i.e., such a molecule is produced by the hand man, e.g., artificially produced through recombinant DNA technology.

Antibodies include proteins that bind to molecules (antigens) via heavy and light chain variable domains, V_{H} and V_{L}, respectively. An antibody is any polyclonal or monoclonal immunoglobulin molecule, or mixture thereof, such as IgM, IgG, IgA, IgE, IgD, and any subclass thereof, such as IgG₁, IgG₂, IgG₃, IgG₄, etc. An antibody also includes a functional (e.g., binding activity) or active (e.g., agonist or antagonist activity) fragment or subsequence, such as, for example, Fab, Fab′, F(ab′)₂, Fv, Fd, single-chain Fv (scFv), disulfide linked Fv, light chain variable (VL) and heavy chain variable (VH) unless otherwise expressly stated.

A monoclonal antibody refers to an antibody that is based upon, obtained from or derived from a single clone, including any eukaryotic, prokaryotic, or phage clone. A “monoclonal” antibody is therefore not defined by the method in which it is produced.

Antibodies are useful for modulating expression or activity of TRAIL (Apo-2L) or TRAIL receptor (DR4 or DR5). Particular antibodies decrease, reduce, inhibit, delay or prevent (antagonist) expression or activity of TRAIL (Apo-2L) or TRAIL receptor (DR4 or DR5), or increase, stimulate, enhance, promote or induce (agonist) expression or activity of TRAIL (Apo-2L) or TRAIL receptor (DR4 or DR5). Antibodies are also useful for detecting TRAIL (Apo-2L) or TRAIL receptor (DR4 or DR5), for example, expressed or secreted by CD8+ T cells, as set forth herein for the screening, identifying and diagnostic methods embodiments.

Antibodies include those specific or selective for binding to TRAIL (Apo-2L) or TRAIL receptor (DR4 or DR5). That is, binding to molecules other than TRAIL (Apo-2L) or TRAIL receptor (DR4 or DR5) is such that the binding does not significantly interfere with detection of TRAIL (Apo-2L) or TRAIL receptor (DR4 or DR5) unless such other molecules have a similar or same epitope the TRAIL (Apo-2L) or TRAIL receptor (DR4 or DR5) that is recognized by the antibody. Selective binding can be distinguished from non-selective binding using specificity, affinity and other binding assays, competitive and non-competitive, known in the art.

Exemplary antibodies include N2B1 and N2B2. Additional non-limiting examples of antibodies are as set forth in Table 2.

Antibodies include “human” forms, which mean that the amino acid sequence of the antibody is fully human or can or do exist in a human antibody. An antibody that is non-human may be made fully human by substituting non-human amino acid residues with amino acid residues that can or do exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk, J. Mol. Biol. 186:651 (1987); Padlan, Mol. Immunol. 31:169 (1994); and Padlan, Mol. Immunol. 28:489 (1991)).

Antibodies include “humanized” forms, which means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Antibodies referred to as “primatized” are within the meaning of “humanized” as used herein, except that the acceptor immunoglobulin molecule and framework region amino acid residues may be any primate amino acid residue (e.g., ape, gibbon, gorilla, chimpanzees orangutan, macaque), in addition to any human residue.

Antibodies include “chimeric” forms, which means that the amino acid sequence of the antibody contains one or more portions that are derived from, obtained or isolated from, or based upon two or more different species. That is, for example, a portion of the antibody may be human (e.g., a constant region) and another portion of the antibody may be non-human (e.g., a murine heavy or light chain variable region). Thus, a chimeric antibody is a molecule in which different portions of the antibody are of different species origins. Unlike a humanized antibody, a chimeric antibody can have the different species sequences in any region of the antibody.

Human antibodies can be produced by immunizing human transchromosomic KM Mice™ (WO 02/43478) or HAC mice (WO 02/092812). KM Mice™ and HAC mice express human immunoglobulin genes. Using conventional hybridoma technology, splenocytes from immunized mice that respond to the antigen can be isolated and fused with myeloma cells. A monoclonal antibody can be obtained that binds to the antigen. An overview of the technology for producing human antibodies is described in Lonberg and Huszar (Int. Rev. Immunol. 13:65 (1995)). Transgenic animals with one or more human immunoglobulin genes (kappa or lambda) that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Mol. Immunol. 28:489 (1991); Studnicka et al., Protein Engineering 7:805 (1994); Roguska et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have been used to humanize antibodies (Carter et al., Proc. Natl. Acad. Sci. USA 89:4285 (1992); and Presta et al., J. Immunol. 151:2623 (1993)).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Protein suitable for generating antibodies can be produced by any of a variety of standard protein purification or recombinant expression techniques known in the art. For example, TRAIL (Apo-2L) or TRAIL receptor (DR4 or DR5) peptides can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Monoclonal antibodies can be readily generated using techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988). Suitable techniques that additionally may be employed in the method including antigen affinity purification, non-denaturing gel purification, HPLC or RP-HPLC, purification on protein A column, or any combination of these techniques. Antibody isotype can be determined using an ELISA assay, for example, a human Ig can be identified using mouse Ig-absorbed anti-human Ig.

Animals which may be immunized include mice, rabbits, rats, sheep, cows or steer, goats, or guinea pigs; such animals include those genetically modified to include human IgG gene loci. Such animals can therefore be used to produce antibodies. Additionally, to increase the immune response, antigen can be coupled to another protein such as ovalbumin or keyhole limpet hemocyanin (KLH), thyroglobulin and tetanus toxoid, or mixed with an adjuvant such as Freund's complete or incomplete adjuvant. Initial and any optional subsequent immunization may be through intraperitoneal, intramuscular, intraocular, or subcutaneous routes. Subsequent immunizations may be at the same or at different concentrations of antigen preparation, and may be at regular or irregular intervals.

TRAIL (Apo-2L) and TRAIL receptor (DR4 or DR5) nucleic acids including antisense and RNAi can modulate expression or activity of TRAIL (Apo-2L) or TRAIL receptor (DR4 or DR5). Antisense includes single, double or triple stranded polynucleotides and peptide nucleic acids (PNAs) that bind RNA transcript or DNA. For example, a single stranded nucleic acid can target TRAIL (Apo-2L) or TRAIL receptor (DR4 or DR5) transcript (e.g., mRNA). Oligonucleotides derived from the transcription initiation site of the gene, e.g., between positions −10 and +10 from the start site, are one particular example. Triplex forming antisense can bind to double strand DNA thereby inhibiting transcription of the gene. “RNAi” is the use of double stranded RNA sequences for inhibiting gene expression (see, e.g., Kennerdell et al., Cell 95:1017 (1998); and Fire et al., Nature, 391:806 (1998)). Double stranded RNA sequences from an TRAIL (Apo-2L) or TRAIL receptor (DR4 or DR5) coding region may therefore be used to inhibit or prevent TRAIL (Apo-2L) or TRAIL receptor (DR4 or DR5) expression or activity.

Antisense and RNAi can be produced based upon the TRAIL (Apo-2L) or TRAIL receptor (DR4 or DR5) sequences known in the art. Exemplary human and mouse TRAIL (Apo-2L) mRNA sequences, respectively, are as follows:

```
(Human, SEQ ID NO: 3)
      1 cctcactgac tataaaagaa tagagaagga agggcttcag tgaccggctg cctggctgac
    61 ttacagcagt cagactctga caggatcatg gctatgatgg aggtccaggg gggacccagc
  121 ctgggacaga cctgcgtgct gatcgtgatc ttcacagtgc tcctgcagtc tctctgtgtg
  181 gctgtaactt acgtgtactt taccaacgag ctgaagcaga tgcaggacaa gtactccaaa
  241 agtggcattg cttgtttctt aaaagaagat gacagttatt gggaccccaa tgacgaagag
  301 agtatgaaca gcccctgctg gcaagtcaag tggcaactcc gtcagctcgt tagaaagatg
  361 attttgagaa cctctgagga aaccatttct acagttcaag aaaagcaaca aaatatttct
  421 cccctagtga gagaaagagg tcctcagaga gtagcagctc acataactgg gaccagagga
  481 agaagcaaca cattgtcttc tccaaactcc aagaatgaaa aggctctggg ccgcaaaata
  541 aactcctggg aatcatcaag gagtgggcat tcattcctga gcaacttgca cttgaggaat
  601 ggtgaactgg tcatccatga aaaagggttt tactacatct attcccaaac atactttcga
  661 tttcaggagg aaataaaaga aaacacaaag aacgacaaac aaatggtcca atatatttac
  721 aaatacacaa gttatcctga ccctatattg ttgatgaaaa gtgctagaaa tagttgttgg
  781 tctaaagatg cagaatatgg actctattcc atctatcaag ggggaatatt tgagcttaag
  841 gaaaatgaca gaatttttgt ttctgtaaca aatgagcact tgatagacat ggaccatgaa
  901 gccagttttt tcggggcctt tttagttggc taactgacct ggaaagaaaa agcaataacc
  961 tcaaagtgac tattcagttt tcaggatgat acactatgaa gatgtttcaa aaaatctgac
1021 caaaacaaac aaacagaaaa cagaaaacaa aaaaacctct atgcaatctg agtagagcag
1081 ccacaaccaa aaaattctac aacacacact gttctgaaag tgactcactt atcccaagaa
1141 aatgaaattg ctgaaagatc tttcaggact ctacctcata tcagtttgct agcagaaatc
1201 tagaagactg tcagcttcca aacattaatg caatggttaa catcttctgt ctttataatc
1261 tactccttgt aaagactgta gaagaaagcg caacaatcca tctctcaagt agtgtatcac
1321 agtagtagcc tccaggtttc cttaagggac aacatcctta agtcaaaaga gagaagaggc
1381 accactaaaa gatcgcagtt tgcctggtgc agtggctcac acctgtaatc ccaacatttt
1441 gggaacccaa ggtgggtaga tcacgagatc aagagatcaa gaccatagtg accaacatag
1501 tgaaacccca tctctactga aagtgcaaaa attagctggg tgtgttggca catgcctgta
1561 gtcccagcta cttgagaggc tgaggcagga gaatcgtttg aacccgggag gcagaggttg
1621 cagtgtggtg agatcatgcc actacactcc agcctggcga cagagcgaga cttggtttca
1681 aaaaaaaaaa aaaaaaaaaa cttcagtaag tacgtgttat ttttttcaat aaaattctat
1741 tacagtatgt caaaaaaaaa aaaaaaaaa
(Mouse, SEQ ID NO: 4)
      1 aactgtgacc ttctcaggca ctgctgctgg gctgcaagtc tgcattggga agtcagacct
    61 ggacagcagt atgccttcct caggggccct gaaggacctc agcttcagtc agcacttcag
  121 gatgatggtg atttgcatag tgctcctgca ggtgctcctg caggctgtgt ctgtggctgt
  181 gacttacatg tacttcacca acgagatgaa gcagctgcag gacaattact ccaaaattgg
  241 actagcttgc ttctcaaaga cggatgagga tttctgggac tccactgatg gagagatctt
  301 gaacagaccc tgcttgcagg ttaagaggca actgtatcag ctcattgaag aggtgacttt
  361 gagaaccttt caggacacca tttctacagt tccagaaaag cagctaagta ctcctccctt
  421 gcccagaggt ggaagacctc agaaagtggc agctcacatt actgggatca ctcggagaag
  481 caactcagct ttaattccaa tctccaagga tggaaagacc ttaggccaga agattgaatc
  541 ctgggagtcc tctcggaaag ggcattcatt tctcaaccac gtgctcttta ggaatggaga
  601 gctggtcatc gagcaggagg gcctgtatta catctattcc caaacatact tccgatttca
  661 ggaagctgaa gacgcttcca agatggtctc aaaggacaag gtgagaacca aacagctggt
  721 gcagtacatc tacaagtaca ccagctatcc ggatcccata gtgctcatga agagcgccag
  781 aaacagctgt tggtccagag atgccgagta cggactgtac tccatctatc agggaggatt
  841 gttcgagcta aaaaaaaatg acaggatttt tgtttctgtg acaaatgaac atttgatgga
  901 cctggatcaa gaagccagct tctttggagc ctttttaatt aactaaatga ccagtaaaga
  961 tcaaacacag ccctaaagta cccagtaatc ttctaggttg aaggcatgcc tggaaagcga
1021 ctgaactggt taggatatgg cctggctgta gaaacctcag gacagatgtg acagaaaggc
1081 agctggaact cagcagcgac aggccaacag tccagccaca gacactttcg gtgtttcatc
1141 gagagacttg ctttctttcc gcaaaatgag atcactgtag cctttcaatg atctacctgg
1201 tatcagtttg cagagatcta gaagacgtcc agtttctaaa tatttatgca acaattgaca
1261 attttcacct ttgttatctg gtccaggggt gtaaagccaa gtgctcacag gctgtgtgca
1321 gaccaggata gctatgaatg caggtcagca taaaaatcac agaatatctc acctaccaaa
1381 tcagagtggg tgtgcccctg tgtgtatatg cgtgtctgtg tgtgtgtgca tgtatgtgtg
1441 tgtgtgtgtg actgttcttt atggtaactg gttatgtttt tctcaagtga aaaacataac
1501 tctatacatg ataacataat atcccatcat cagtggaacc ttgcccaaag aatgtatgaa
1561 atctccaggc aatgaatgag ggcagcccaa gaaagaggcc cgcagagcca taccacaggg
1621 ctgccccacc ctgctggagc tcagatcctg ccactgctgc aggccctggg taccaggtgt
1681 agagttggag gaggtcttgc ctgtgggtct aggtctttgg tgcctacctc cttgatatgg
1741 ccccagtcct cctttgcttg tttgctagtt ttatcatgtt tcccaggccg gcctcaagtc
1801 caatatgtag tcaagagtga tctctaactg tgcaacctcc tgcctccaag atctgctgag
1861 attataggca tgtgcccccc tgtctgattt gtgtagagcc aggcttcttg tacatgtgac
1921 aaccatgcca ccctcagctc tgtcccagct ccatttcttc ctttctgaat gcaagcattt
1981 actttgtgtc cctatattct agaatgtgca acagtgaaga atttgctctg actttcagga
2041 taaagtttga actaggttca ccatgcttgc tttgtccaga ttgcgactgt cacccagtcc
2101 tctggctctt ccatctgtct gtccactcca cctaccaaga tgttgaacac ttgttctttt
2161 taagatgttg gtgcctggag tttcattaga gtaacacaaa actaactaaa accaaacaac
2221 tccaaaggag cccatatgtg ttttaatgaa acatttttta agcctattgg gggcctgaag
2281 agattgctca gaggaaaaca gcacttccag aggacccagg ttcaattctc atcgctgatg
2341 tgatagttaa cagctgtaac ttcagttcca aggggtctga ctttctgccc tttgcttgca
2401 atgcatgtat gtgatacaca gacatacatt ctgacaaaat atatccatac acaaaagtat
2461 ttttttaaaa gcttatttga atgtaagagt atggctagct gtcacttctg ataccccttc
2521 ttattttttt atgactcaag cccttataaa ctagcaaata gaagtcacag ctaccacttg
2581 aatataagca cttgaatacc tcctctcact agaatacaac atagcttaat agtaaaaatc
2641 ttgccttagt aaagtacttg catgtcatgt ctacatgaac caaatgaatg tattaattaa
2701 taatagacat aatgatcaca tcggaaaggc tgtgagaaat aatggagaac atttgaaagc
2761 tcaagatgga agggaaaggc acttgtcaaa aatcttgaca acctgaattt gacctttggc
2821 agggctgaaa actaaaccca gggtcttact cccagtaggc atgaaccccc acactgagcg
2881 gcaccacagc cctaaggttt taaatggcag aaacaactga gcgcttttct aattcctggt
2941 ccccaagtca tcactgatgg caaaattcac agcacttcat cttgtctgcc aagctggcaa
3001 gcgaaatggt gtctgatttc ttttttgttt ttttaaagat ttattcatta ttatatctaa
3061 gtacactgta gctgtcttca gatgcaccag aaaagggcgt cagatctcat tacggatggt
3121 tgtgagccac catgtggttg ctgggatttg aactcagaaa cttcagaaga gcagtcggtg
3181 ctcttaacca ctgagccacc tctccagccc cagtgtctga tttctaatgt ttgtgttgaa
3241 cgtatacatt ctttgtctgc tgttaattct atagtaatct cctgtgaaag attttataac
3301 gcccaggcct ggattcaacc acaatccaac aggatcggaa tcctggggtg gtgctcagag
3361 atctttttgt gcgtgcattt tagtttttta aagtaattag caggcagcct gcaggacaca
3421 tctttgccaa aatgtcaaat gctgtgactt ataaattaaa tatttaagaa tgcagcatac
3481 ccagagaaat cattagatgt tcaaagattt aattcgtgtg caacattaaa tgccatgact
3541 tgtttaaaca agcacaccta aatattcttt cttctgctag aatctgaagc ctgtgatgca
3601 ggaagcaaga atggagagaa tattctagaa taagaacttt ctgggcgatt gtgagccctt
3661 taataaaagg catgaatact tatctgtttg aaagagaaat caatttgggg tgggggcggg
3721 ggttgtcctt tctttgctga gttctagccc aacaagccat ctgacttgaa gactaagacg
3781 agcctgccac ctgggcaagc acagggacaa tgatcccagg gcagaggcca gctctgctgt
3841 tttgagaagt gtctaggcaa aagctttctt tctttttttt tttttttcag accaaaaggg
3901 gcctctttaa tgtcacacac cattagtgcc accaggtggc gaggttccct taatttcttc
3961 agtatcaagt cttttaaaag tgtatgagta taaacatctt caaattctcg ggtttaaaaa
4021 caaaatcaaa aatgaaaaaa ggtcgcttac ctttagacca taggcaactg gagctaaaga
4081 caagagataa gctggtgtac cccgatggag cagcaggctt taactgaatc tgtgaggaac
4141 agggtggggc ttcacatggt aaaactgaat ctgaggtgtg ttcttttgcc tgtttgggtt
4201 gttgctgttg ttactgcttt gttttgtttt tttgttttta agtgaaacag aatttaagtc
4261 ttttggaaga cttttgtaaa ttctcttggt cccaagggag ggctctgtga cgtctggagt
4321 ctcaacagtt tggtctttgc cctgtctcct gttattatag actgtggcct ttgccactcc
4381 atcactgcct actgtttttc ttggagacag ggtctctccc attacgggca gccagcagtg
4441 ccctgagagt ctcttgacat gtggccatga aggcttttta caaggatgct ggggatttga
4501 acccgggttc tcatgcttga gccataactg ctcttaccct agaactgtct ccccagtcca
4561 atcactgcta ctattttttt ttcttggcaa atcaattcag ttctttagtt ttctttcaag
4621 tattgtttta cctatttatc atatttatca ctttcattat caatgttaga aattttgttt
4681 agccagcctt tttacttatt cttgaaataa agggttaaat agatattttt tcagttgtga
4741 gacatgaaaa tcttagaaag aatttttctt ttctttttcc tttcatttta atagcagtga
4801 atctagaaag agaattattt ttcatggtga ttatttcaaa tgtattgata ccattttgtg
4861 ggctgggaaa tgttaaactt tgtaaactct gaaacgcaca aagtgttgct ttgaatttca
4921 ccttaataaa aataacatca agta
```

Exemplary human and mouse TRAIL receptor (DR4 or DR5) mRNA sequences, respectively, are as follows:

```
(Human NFRSF10A, TRAILR-1 (DR4), SEQ ID NO: 5)
      1 atggcgccac caccagctag agtacatcta ggtgcgttcc tggcagtgac tccgaatccc
    61 gggagcgcag cgagtgggac agaggcagcc gcggccacac ccagcaaagt gtggggctct
  121 tccgcgggga ggattgaacc acgaggcggg ggccgaggag cgctccctac ctccatggga
  181 cagcacggac ccagtgcccg ggcccgggca gggcgcgccc caggacccag gccggcgcgg
  241 gaagccagcc ctcggctccg ggtccacaag accttcaagt ttgtcgtcgt cggggtcctg
  301 ctgcaggtcg tacctagctc agctgcaacc atcaaacttc atgatcaatc aattggcaca
  361 cagcaatggg aacatagccc tttgggagag ttgtgtccac caggatctca tagatcagaa
  421 cgtcctggag cctgtaaccg gtgcacagag ggtgtgggtt acaccaatgc ttccaacaat
  481 ttgtttgctt gcctcccatg tacagcttgt aaatcagatg aagaagagag aagtccctgc
  541 accacgacca ggaacacagc atgtcagtgc aaaccaggaa ctttccggaa tgacaattct
  601 gctgagatgt gccggaagtg cagcacaggg tgccccagag ggatggtcaa ggtcaaggat
  661 tgtacgccct ggagtgacat cgagtgtgtc cacaaagaat caggcaatgg acataatata
  721 tgggtgattt tggttgtgac tttggttgtt ccgttgctgt tggtggctgt gctgattgtc
  781 tgttgttgca tcggctcagg ttgtggaggg gaccccaagt gcatggacag ggtgtgtttc
  841 tggcgcttgg gtctcctacg agggcctggg gctgaggaca atgctcacaa cgagattctg
  901 agcaacgcag actcgctgtc cactttcgtc tctgagcagc aaatggaaag ccaggagccg
  961 gcagatttga caggtgtcac tgtacagtcc ccaggggagg cacagtgtct gctgggaccg
1021 gcagaagctg aagggtctca gaggaggagg ctgctggttc cagcaaatgg tgctgacccc
1081 actgagactc tgatgctgtt ctttgacaag tttgcaaaca tcgtgccctt tgactcctgg
1141 gaccagctca tgaggcagct ggacctcacg aaaaatgaga tcgatgtggt cagagctggt
1201 acagcaggcc caggggatgc cttgtatgca atgctgatga aatgggtcaa caaaactgga
1261 cggaacgcct cgatccacac cctgctggat gccttggaga ggatggaaga gagacatgca
1321 aaagagaaga ttcaggacct cttggtggac tctggaaagt tcatctactt agaagatggc
1381 acaggctctg ccgtgtcctt ggagtga
(Human TNERSF10B, TRAILR-2 (DR5), SEQ ID NO: 6)
      1 atggaacaac ggggacagaa cgccccggcc gcttcggggg cccggaaaag gcacggccca
    61 ggacccaggg aggcgcgggg agccaggcct gggccccggg tccccaagac ccttgtgctc
  121 gttgtcgccg cggtcctgct gttggtctca gctgagtctg ctctgatcac ccaacaagac
  181 ctagctcccc agcagagagc ggccccacaa caaaagaggt ccagcccctc agagggattg
  241 tgtccacctg gacaccatat ctcagaagac ggtagagatt gcatctcctg caaatatgga
  301 caggactata gcactcactg gaatgacctc cttttctgct tgcgctgcac caggtgtgat
  361 tcaggtgaag tggagctaag tccctgcacc acgaccagaa acacagtgtg tcagtgcgaa
  421 gaaggcacct tccgggaaga agattctcct gagatgtgcc ggaagtgccg cacagggtgt
  481 cccagaggga tggtcaaggt cggtgattgt acaccctgga gtgacatcga atgtgtccac
  541 aaagaatcag gcatcatcat aggagtcaca gttgcagccg tagtcttgat tgtggctgtg
  601 tttgtttgca agtctttact gtggaagaaa gtccttcctt acctgaaagg catctgctca
  661 ggtggtggtg gggaccctga gcgtgtggac agaagctcac aacgacctgg ggctgaggac
  721 aatgtcctca atgagatcgt gagtatcttg cagcccaccc aggtccctga gcaggaaatg
  781 gaagtccagg agccagcaga gccaacaggt gtcaacatgt tgtcccccgg ggagtcagag
  841 catctgctgg aaccggcaga agctgaaagg tctcagagga ggaggctgct ggttccagca
  901 aatgaaggtg atcccactga gactctgaga cagtgcttcg atgactttgc agacttggtg
  961 ccctttgact cctgggagcc gctcatgagg aagttgggcc tcatggacaa tgagataaag
1021 gtggctaaag ctgaggcagc gggccacagg gacaccttgt acacgatgct gataaagtgg
1081 gtcaacaaaa ccgggcgaga tgcctctgtc cacaccctgc tggatgcctt ggagacgctg
1141 ggagagagac ttgccaagca gaagattgag gaccacttgt tgagctctgg aaagttcatg
1201 tatctagaag gtaatgcaga ctctgccatg tcctaa
(Mouse Tnfrsf10b, TRAIL-R2 (DR5), SEQ ID NO: 7)
      1 gtcgccgcga caagaatcca gaacttttct gggagtgagg aaatccagag aactttttta
    61 ggagtgaggg gacagccatc cttcgtggct tttgggagct gaagccgcag ggtttcggat
  121 gagctgacac catggagcct ccaggaccca gcacgcccac agcctctgcc gctgcccggg
  181 cagatcacta caccccaggc ctccggccac tcccgaagcg cagacttcta tatagctttg
  241 cgttgctgct tgctgtgcta caggctgtct ttgttccagt aacagctaac ccagcccata
  301 atcgtccagc tggcctacag cggccggagg agagcccatc aagaggaccc tgtctagcag
  361 gccagtacct gtcagaaggg aactgcaagc cttgcagaga gggtattgac tacaccagcc
  421 attccaacca ttctctggat tcatgtattc tctgcacagt ctgtaaggaa gataaagtcg
  481 tagaaacccg atgcaacata accacaaata cggtgtgtcg atgcaaacca ggcacctttg
  541 aagataaaga ctcccctgag atctgccagt catgctctaa ctgcactgac ggggaagagg
  601 aactgacttc ctgtaccccc agagaaaacc ggaagtgtgt ctccaaaacg gcttgggcat
  661 cttggcataa gctaggcctc tggataggac tcctggttcc agtagtgctg ctgattggag
  721 ctctgcttgt ctggaagact ggagcatgga ggcaatggtt gctctgtata aaaagaggct
  781 gtgaacggga tcccgaaagt gcgaactctg tgcattcgtc tctcttggac cgacagacat
  841 ctagcacgac aaatgactct aaccacaaca cggaacctgg caagactcag aaaacaggaa
  901 agaagttgct ggttccggta aacggaaacg actcagctga cgacctgaag tttatcttcg
  961 agtattgttc ggacatagtg ccctttgact cctggaaccg tctcatgcgg cagttgggcc
1021 tcacagacaa tcaaatccaa atggtcaaag ccgaaacact ggtcacacgt gaggccctgt
1081 accaaatgct gctcaagtgg cgccaccaga ctgggcgaag tgcctccatc aaccatctgc
1141 tggatgcctt ggaagccgtg gaagagagag atgccatgga gaaaattgaa gactacgcag
1201 tgaaatccgg gaggtttact tatcagaacg ctgcagccca accagagaca gggccaggag
1261 gatctcagtg cgtttgaagt cagcctgatc tacttagtga actcaggaca gccaaggcta
1321 tgtagagagc cccgaagatg caggctcttc agtattatga gaatgtactt aattttttct
1381 tgtagtagtt agtgtatcat attattgtat tatttatatt attactgtta agtactatgt
1441 tctcttatta gaagttgaac acagaacctc tgagaacaca tatgctacaa gtgttctaac
1501 acacctccag catcccggat tacctttgtt cctgaacaag gcacaattgg tagggtatga
1561 tagggcctgc ctatcatcct aacactccgg tgatggagcc aggaagatca agagttcgag
1621 gccagctggt tcacataaga tcccatataa tgtgcaggat ggctaaactt gctgagagct
1681 gactctgtgg tctcctgtcc cagattctag cgatattcat tactaagacc cttgtccaga
1741 gacaaaagac cacctctgta acagagggaa gaataaaaca gccctagggt ggaaactcct
1801 tgtgaacaca gccactgctg tttactgtta gactactgct cagcactaca cagctgcacg
1861 gcacctccct gtgccaggtg ctagtgggca gcctactgag ggtacatcta acttgaatct
1921 aacacacttg aagtgagttt gctggtttgg acacagaggg agcattaagt gctacctggg
1981 gtgacccttg aggaccacgc cccctgtaag catttgacca ttgtgagagt aaacactgaa
2041 actcaccatt gtcctgcctc agcctttcta gagctggaat cataggtatg ctgcaccgga
2101 tccagaagga gaaataacta cctttagata ctgtgatagg gatttctaga aagctgccac
2161 atacagattt ttgtcctgtg tgaattccta ttgttttttg tttgttttta attttttatt
2221 atttattttc ttcattcaca tttcaaatgc tatcccaaaa gtcccctatg ccctcccccc
2281 ccctgccccc cgccctgctc ccctacccac tcactcactc ccactttttt tttttttttt
2341 gagacagggt ttctctgtgt agccctggct gtcctggaac tcactctgta gaccaggctg
2401 gcctcgaact cagaaattca cctgcctctg cctcccaagt gctgggatta aaggtgtgtg
2461 ccaccatgcc cggccttttt tttttttttt tttaattagg tatttatttc atttaaattt
2521 ccaatgctat cccaaaagtc ccccacatgc tcccccaccc actccccacc tgactttata
2581 tgcctcactc ccacttcttg gccctggtgt tcccctgtac tggggcatat aacgtttgca
2641 agaccaaggg gcgtctcttt ccactgatgg ccgactaggc catcttctga tacatatgca
2701 gctagagaca cgagctctgg gggggtactg gttagttcat attgttgttc cacctatagg
2761 gttgccgacc ccttcagctc cttgggtact ttctctagct cctccactag gggccctgtg
2821 ttctatccaa tagatgactg tgagcatcca cttctgtatt tgccaggcac tggcatagcc
2881 tcatacgaga cagctatatc agggtccagt tttgttttgt tttgttttaa actagcatgc
2941 tggagaggta gctcagcggt taagagcagt ggctgctctt ccagaggtcc tgagttccaa
3001 ttcccagcag ctacatagtg gctcacaacc ttctctaatg ggatctgatg tccttttcta
3061 gtgtgtttga agccagtggc agtgtaatta catacacaaa ataaataaat ctttttaaaa
3121 aagaaaaaaa aaaaaaaaaa aa
```

Dominant negative molecules that can directly or indirectly modulate TRAIL (Apo-2L) or TRAIL receptor (DR4 or DR5) signaling include decoy TRAIL receptors. Such receptors are capable of binding TRAIL (Apo-2L) but do not transmit the signal. Such receptors are therefore useful for inhibiting TRAIL (Apo-2L) activity. Exemplary human TRAIL decoy receptor mRNA sequences are as follows:

```
(Human NFRSF10C, TRAILR-3 (DCR1), SEQ ID NO: 8)
      1 gctgtgggaa cctctccacg cgcacgaact cagccaacga tttctgatag atttttggga
    61 gtttgaccag agatgcaagg ggtgaaggag cgcttcctac cgttagggaa ctctggggac
  121 agagcgcccc ggccgcctga tggccgaggc agggtgcgac ccaggaccca ggacggcgtc
  181 gggaaccata ccatggcccg gatccccaag accctaaagt tcgtcgtcgt catcgtcgcg
  241 gtcctgctgc cagtcctagc ttactctgcc accactgccc ggcaggagga agttccccag
  301 cagacagtgg ccccacagca acagaggcac agcttcaagg gggaggagtg tccagcagga
  361 tctcatagat cagaacatac tggagcctgt aacccgtgca cagagggtgt ggattacacc
  421 aacgcttcca acaatgaacc ttcttgcttc ccatgtacag tttgtaaatc agatcaaaaa
  481 cataaaagtt cctgcaccat gaccagagac acagtgtgtc agtgtaaaga aggcaccttc
  541 cggaatgaaa actccccaga gatgtgccgg aagtgtagca ggtgccctag tggggaagtc
  601 caagtcagta attgtacgtc ctgggatgat atccagtgtg ttgaagaatt tggtgccaat
  661 gccactgtgg aaaccccagc tgctgaagag acaatgaaca ccagcccggg gactcctgcc
  721 ccagctgctg aagagacaat gaacaccagc ccagggactc ctgccccagc tgctgaagag
  781 acaatgacca ccagcccggg gactcctgcc ccagctgctg aagagacaat gaccaccagc
  841 ccggggactc ctgccccagc tgctgaagag acaatgacca ccagcccggg gactcctgcc
  901 tcttctcatt acctctcatg caccatcgta gggatcatag ttctaattgt gcttctgatt
  961 gtgtttgttt gaaagacttc actgtggaag aaattccttc cttacctgaa aggttcaggt
1021 aggcgctggc tgagggcggg gggcgctgga cactctctgc cctgcctccc tctgctgtgt
1081 tcccacagac agaaacgcct gcccctgccc caaaaaaaaa aaaaaaaaaa aaaaaaaaaa
1141 aaaaaaaaaa aaaaaaaaaa aaaaaaaaaa aaaaaaaaaa
(Human TNFRSF10D, TRAILR-4 (DCR2), SEQ ID NO: 9)
      1 cgagaacctt tgcacgcgca caaactacgg ggacgatttc tgattgattt ttggcgcttt
    61 cgatccaccc tcctcccttc tcatgggact ttggggacaa agcgtcccga ccgcctcgag
  121 cgctcgagca gggcgctatc caggagccag gacagcgtcg ggaaccagac catggctcct
  181 ggaccccaag atccttaagt tcgtcgtctt catcgtcgcg gttctgctgc cggtccgggt
  241 tgactctgcc accatccccc ggcaggacga agttccccag cagacagtgg ccccacagca
  301 acagaggcgc agcctcaagg aggaggagtg tccagcagga tctcatagat cagaatatac
  361 tggagcctgt aacccgtgca cagagggtgt ggattacacc attgcttcca acaatttgcc
  421 ttcttgcctg ctatgtacag tttgtaaatc aggtcaaaca aataaaagtt cctgtaccac
  481 gaccagagac accgtgtgtc agtgtgaaaa aggaagcttc caggataaaa actcccctga
  541 gatgtgccgg acgtgtagaa cagggtgtcc cagagggatg gtcaaggtca gtaattgtac
  601 gccccggagt gacatcaagt gcaaaaatga atcagctgcc agttccactg ggaaaacccc
  661 agcagcggag gagacagtga ccaccatcct ggggatgctt gcctctccct atcactacct
  721 tatcatcata gtggttttag tcatcatttt agctgtggtt gtggttggct tttcatgtcg
  781 gaagaaattc atttcttacc tcaaaggcat ctgctcaggt ggtggaggag gtcccgaacg
  841 tgtgcacaga gtccttttcc ggcggcgttc atgtccttca cgagttcctg gggcggagga
  901 caatgcccgc aacgagaccc tgagtaacag atacttgcag cccacccagg tctctgagca
  961 ggaaatccaa ggtcaggagc tggcagagct aacaggtgtg actgtagagt cgccagagga
1021 gccacagcgt ctgctggaac aggcagaagc tgaagggtgt cagaggagga ggctgctggt
1081 tccagtgaat gacgctgact ccgctgacat cagcaccttg ctggatgcct cggcaacact
1141 ggaagaagga catgcaaagg aaacaattca ggaccaactg gtgggctccg aaaagctctt
1201 ttatgaagaa gatgaggcag gctctgctac gtcctgcctg tgaaagaatc tcttcaggaa
1261 accagagctt ccctcattta ccttttctcc tacaaaggga agcagcctgg aagaaacagt
1321 ccagtacttg acccatgccc caacaaactc tactatccaa tatggggcag cttaccaatg
1381 gtcctagaac tttgttaacg cacttggagt aatttttatg aaatactgcg tgtgataagc
1441 aaacgggaga aatttatatc agattcttgg ctgcatagtt atacgattgt gtattaaggg
1501 tcgttttagg ccacatgcgg tggctcatgc ctgtaatccc agcactttga taggctgagg
1561 caggtggatt gcttgagctc gggagtttga gaccagcctc atcaacacag tgaaactcca
1621 tctcaattta aaaagaaaaa aagtggtttt aggatgtcat tctttgcagt tcttcatcat
1681 gagacaagtc tttttttctg cttcttatat tgcaagctcc atctct
```

The term “subject,” also referred to as “patient,” means an animal, typically mammalian animal, such as but not limited to non-human primates (apes, gibbons, gorillas, chimpanzees, orangutans, macaques), domestic animals (dogs and cats), a farm animals (chickens, ducks, horses, cows, goats, sheep, pigs), experimental animal (mouse, rat, rabbit, guinea pig) and humans. Subjects include animal models, for example, a model of autoimmunity (BXSB mouse for lupus, experimental autoimmune encephalomyelitis (EAE) for multiple sclerosis, rheumatoid arthritis and inflammatory bowel disease, NOD mouse for insulin-dependent diabetes, collagen induced arthritis (CIA) for rheumatoid arthritis, etc.), immunosuppression (Nude mice), HIV infection (e.g., a primate SIV model). Subjects include naturally occurring or non-naturally occurring mutated or non-human genetically engineered (e.g., transgenic or knockout) animals.

Subjects include animals having or at risk of having a chronic or acute physiological condition, disorder, illness, disease or symptom as set forth herein. Subjects can be any age. For example, a subject (e.g., human) can be a newborn, infant, toddler, child, teenager, adult, or elderly e.g., 50 years or older.

Subjects include those in need of or those that may benefit from a method embodiment, e.g., in need of or benefit from a therapeutic or prophylactic treatment. A subject is considered to be in need of a method of the invention where a method may provide an objective or subjective benefit to the subject. Various benefits or improvement provided to a subject by various methods embodiments are as set forth herein and known in the art for the various physiological conditions, disorders, illnesses, diseases and symptoms set forth herein.

Subjects further include immunocompromised subjects due to an immunological condition, disorder, illness, disease (e.g., autoimmunity) or symptom, or an immune-suppressing condition, disorder, illness, disease, treatment or therapy (e.g., cyclophosphamide). Subjects therefore include those with undesirable or abnormal low or reduced numbers of CD8+ T cells (e.g., CD8+ T cells, CD8+ T cells primed in the absence of CD4+ T cell help, antigen-specific CD8+ T cells that produce TRAIL upon antigen re-encounter), and those with undesirable or abnormal low or reduced numbers of CD4+ T cells or CD8+ T cells. Appropriate subjects therefore also include those having reduced numbers of CD4+ T cells, those having reduced numbers of CD8+ T cells due to activation-induced cell death or apoptosis (e.g., CD8+ T cells primed in the absence of CD4+ T cell help, antigen-specific CD8+ T cells that produce TRAIL upon antigen re-encounter), those in need of stabilizing or increasing or inhibiting a progressive reduction or loss of CD8+ (naïve or CD8+ T cells primed in the absence of CD4+ T cell help, antigen-specific CD8+ T cells that produce TRAIL upon antigen re-encounter) or CD4+ T cell numbers or activity, as set forth herein, regardless of the reason for the need. As a non-limiting example, a subject in need of treatment would include subjects having a CD4+ T cell count less than 600, 500, 400, 300 cells/cubic millimeter (mm3) blood, or less than 200 cells/cubic millimeter (mm3) blood, or the percentage of CD4+ T cells in the subject is less than 40%, 25%, or 15% of all lymphocytes, regardless of the reason why the subject has reduced CD4+ T cell numbers.

Subjects also include those receiving or candidates for a cellular, tissue or organ transplant. Such subjects can exhibit an undesirable or aberrant immune response that leads to destruction of a transplanted cell(s), tissue or organ, as in graft vs. host disease. Treating such a subject in accordance with a method embodiment can result in reducing, inhibiting or preventing damage to or rejection of transplanted cell, tissue or organ.

For HIV, appropriate subjects include those having HIV infection or pathogenesis or having any symptom or pathology associated with or caused by HIV. Target subjects therefore include subjects that have been infected with HIV, have been diagnosed as HIV+, or that have developed one or more adverse symptoms or pathologies associated with or caused by HIV infection or pathogenesis (e.g., illness), regardless of the virus type, timing or degree of onset, progression, severity, frequency, duration of any infection, pathogenesis (e.g., illness), symptom, pathology or adverse side effect.

Subjects also include subjects those having antigen-specific CD8+ T cells (e.g., specific for a bacterial, viral (e.g., HIV or hepatitis antigen), fungal, parasite, tumor or cancer antigen), and which CD8+ T cells optionally produce or express TRAIL. For example, a subject in need of treatment would be a subject with relative low or reduced CD4+ T cell numbers or activity, which as a consequence CD8+ T cells primed with an antigen in the absence of CD4+ T cell help produce TRAIL upon antigen re-encounter. Subjects further include those having reduced numbers of CD4+ T cells, as compared to an age, gender, race, etc. matched subject. Subjects additionally include those suffering from a progressive reduction or loss of CD4+ T cells over a period of time. For example, a subject in need of treatment would include an immunosuppressed (e.g., HIV+) subject having a CD4+ T cell count less than 600, 500, 400, 300 cells/cubic millimeter (mm3) blood, or less than 200 cells/cubic millimeter (mm3) blood, or the percentage of CD4+ T cells in the subject is less than 40%, 25%, or 15% of all lymphocytes.

Subjects appropriate for treatment also include those at risk of HIV infection or pathogenesis or at risk of having or developing an HIV infection. Candidate subjects therefore include subjects that have been exposed to or contacted with HIV, or that are at risk of exposure to or contact with HIV, regardless of the type, timing or extent of exposure or contact. The invention methods are therefore applicable to a subject who is at risk of HIV infection or pathogenesis, but has not yet been exposed to or contacted with HIV. Prophylactic methods are therefore included. Subjects targeted for prophylaxis can be at increased risk (probability or susceptibility) of HIV infection or pathogenesis, as set forth herein and known in the art.

At risk subjects appropriate for treatment include subjects exposed to other subjects having HIV, or where the risk of HIV infection is increased due to changes in virus infectivity or cell tropism, immunological susceptibility (e.g., an immunocompromised subject), or environmental risk. At risk subjects appropriate for treatment therefore include human subjects exposed to or at risk of exposure to other humans that have an HIV infection (e.g., diagnosed as HIV+)

Subjects also appropriate for treatment include those vaccinated or immunized with an antigen or a candidate for immunization or vaccination with an antigen. Such subjects include vaccination or immunization with antigens of infectious agents, such as microorganisms (bacteria, viral, fungal, parasite or prion), as well as antigens present in disease, such as tumors and cancers (e.g., tumor or cancer associated antigens). A particular subject for immunization or vaccination includes those with CD8+ T cells primed in the absence of CD4+ T cell help, or antigen-specific CD8+ T cells that produce TRAIL upon antigen re-encounter. Such a subject is or may be a candidate for administration of an anti-microbial.

In the non-limiting example of HIV subjects, such subjects include candidates for and those immunized or vaccinated with any antigen (microorganism or disease), including live or attenuated HIV. Immunized or vaccinated subjects can but need not have been exposed to or contacted with the etiological agent (e.g., microorganism or disease) to which the antigen is intended to elicit an immunological response.

Subjects appropriate for treatment include those having or at risk of having a tumor or cancer (metastatic or non-metastatic), as set forth herein or known in the art. Subjects appropriate for treatment also include those undergoing or having undergone anti-tumor or -cancer therapy, including subjects where the tumor or cancer is in remission. Embodiments therefore include treating a subject who is at risk of a tumor or cancer, or a complication associated with a tumor or cancer, for example, due to tumor reappearance or re-growth following tumor or cancer reduction or remission.

“At risk” subjects include those having risk factors associated with undesirable or aberrant immune response, immune disorder or immune disease, development of cell-proliferative or hyperproliferative condition, disorders, disease or illness (e.g., a tumor), or exposure to or contact with an infectious agent (e.g., HIV). Risk factors include gender, lifestyle (diet, smoking), occupation (medical and clinical personnel), environmental factors (carcinogen exposure), family history (autoimmune disorders, diabetes, etc.), genetic predisposition, etc. For example, subjects at risk for developing melanoma include excess sun exposure (ultraviolet radiation), fair skin, high numbers of naevi (dysplastic nevus), patient phenotype, family history, or a history of a previous melanoma. Subjects at risk for developing cancer can therefore be identified by lifestyle, occupation, environmental factors, family history, and genetic screens for tumor associated genes, gene deletions or gene mutations. Subjects at risk for developing breast cancer lack Brca1, for example. Subjects at risk for developing colon cancer have early age or high frequency polyp formation, or deleted or mutated tumor suppressor genes, such as adenomatous polyposis coli (APC), for example. Subjects at risk for immunodeficiency with hyper-IgM (HIM) have a defect in the gene TNFSF5, found on chromosome X at q26, for example. Susceptibility to autoimmune disease is frequently associated with MHC genotype. For example, in diabetes there is an association with HLA-DR3 and HLA-DR4.

Subjects appropriate for treatment further include those having or at risk of having a chronic or acute bacterial, viral, fungal, parasite or prion infection. Exemplary viral infections include, for example, HIV and hepatitis (e.g., hepatitis A, B, C, D or G).

Embodiments include pharmaceutical compositions/formulations, which are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. A formulation is compatible in that it does not destroy activity of an active ingredient therein (e.g., a molecule that binds to and modulates TRAIL (Apo-2L) or TRAIL receptor (DR4 or DR5) expression or activity, such as an inhibitor (e.g., antagonist), or an activator (e.g., agonist)), or induce adverse side effects that far outweigh any prophylactic or therapeutic effect or benefit.

Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

The formulations may, for convenience, be prepared or provided as a unit dosage form. Preparation techniques include bringing into association the active ingredient and a pharmaceutical carrier(s) or excipient(s). In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient (e.g., a molecule that binds to and modulates TRAIL (Apo-2L) or TRAIL receptor (DR4 or DR5) expression or activity, such as an inhibitor (e.g., antagonist), or an activator (e.g., agonist)) in a free-flowing form such as a powder or granules, optionally mixed with a binder, lubricant, inert diluent, preservative, surface-active or dispersing agent. Molded tablets may be produced by molding, in a suitable apparatus, a mixture of powdered compound (e.g., a molecule that binds to and modulates TRAIL (Apo-2L) or TRAIL receptor (DR4 or DR5) expression or activity, such as an inhibitor (e.g., antagonist), or an activator (e.g., agonist)) moistened with an inert liquid diluent. The tablets may optionally be coated or scored and may be formulated so as to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include those set forth above and, nevirapine, delavirdine, efavirenz, saquinavir, ritonavir, indinavir, nelfinavir, amprenavir, zidovudine (AZT), stavudine (d4T), lamivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenic alkanolamide, ciclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, ketoconazole, undecylenate, silver sulfadiazine, undecylenic acid, undecylenic alkanolamide and Carbol-Fuchsin.

Pharmaceutical compositions can optionally be formulated to be compatible with a particular route of administration. Thus, pharmaceutical compositions include carriers (excipients, diluents, vehicles or filling agents) suitable for administration by various routes and delivery, locally, regionally or systemically, ex vivo or in vivo.

Exemplary routes of administration for contact or ex vivo or in vivo delivery include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, intralymphatic.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be in unit-dose or multi-dose kits, for example, ampules and vials, and may be stored in a freeze-dried (lyophilized) condition requiring addition of a sterile liquid carrier, for example, water for injections.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils or creams as generally known in the art.

For topical administration, for example, to skin, pharmaceutical compositions typically include ointments, creams, lotions, pastes, gels, sprays, aerosols or oils. Carriers which may be used include Vaseline, lanolin, polyethylene glycols, alcohols, transdermal enhancers, and combinations thereof. An exemplary topical delivery system is a transdermal patch containing an active ingredient (e.g., a molecule that binds to and modulates TRAIL (Apo-2L) or TRAIL receptor (DR4 or DR5) expression or activity, such as an inhibitor (e.g., antagonist), or an activator (e.g., agonist)).

For oral administration, pharmaceutical compositions include capsules, cachets, lozenges, tablets or troches, as powder or granules. Oral administration formulations also include a solution or a suspension (e.g., aqueous liquid or a non-aqueous liquid; or as an oil-in-water liquid emulsion or a water-in-oil emulsion).

For rectal administration, pharmaceutical compositions can be included as a suppository with a suitable base comprising, for example, cocoa butter or a salicylate. For vaginal administration, pharmaceutical compositions can be included as pessaries, tampons, creams, gels, pastes, foams or spray formulations containing in addition to the active ingredient (e.g., a molecule that binds to and modulates TRAIL (Apo-2L) or TRAIL receptor (DR4 or DR5) expression or activity, such as an inhibitor (e.g., antagonist), or an activator (e.g., agonist)) a carrier, examples of appropriate carriers which are known in the art.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Embodiments including pharmaceutical formulations can be packaged in unit dosage forms for ease of administration and uniformity of dosage. A “unit dosage faun” as used herein refers to a physically discrete unit suited as unitary dosages for treatment or administration; each unit containing a predetermined quantity of compound optionally in association with a pharmaceutical carrier (excipient, diluent, vehicle or filling agent) which, when administered in one or more doses, is calculated to produce a desired effect (e.g., a desired effect or benefit). Unit dosage forms can contain a daily dose or unit, daily sub-dose, or an appropriate fraction thereof, of an administered molecule (e.g., a molecule that binds to and modulates TRAIL (Apo-2L) or TRAIL receptor (DR4 or DR5) expression or activity, such as an inhibitor (e.g., antagonist), or an activator (e.g., agonist)). Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

Methods embodiments include contact or administration in vitro, ex vivo and in vivo at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo or in vivo delivery can be dictated by the physiological condition, disorder, illness, disease or symptom to be treated. For example, an amount can be administered to the subject substantially contemporaneously with, or within about 1-60 minutes, hours or days of the onset of a symptom of an immunosuppressive condition, disorder, illness, disease or symptom (e.g., HIV infection), or a vaccination.

Compositions and methods embodiments include contact or administration in vitro, ex vivo or in vivo. Compositions and methods embodiments may be practiced via systemic, regional or local administration, by any route. Compositions and methods embodiments may be administered as a single or multiple doses to provide the intended effect.

A subject may be administered in single bolus or in divided/metered doses, which can be adjusted to be more or less according to the various considerations set forth herein and known in the art. Doses may vary depending upon the physiological condition, disorder, illness, disease or symptom to be treated, the onset, progression, severity, frequency, duration, probability of or susceptibility of the physiological condition, disorder, illness, disease or symptom to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome or beneficial effect desired, status of the physiological condition, disorder, illness, disease or symptom, any adverse side effects of the treatment or therapy, etc. For example, once control or a particular endpoint is achieved, for example, dose amount, frequency or duration can be reduced. The skilled artisan will appreciate the factors that may influence the dosage, frequency and timing required to provide an amount sufficient or effective for treatment.

For therapeutic treatment, a method is performed as soon as practical, typically within 0-72 hours or days after a subject is exposed to, contacted or manifests the physiological condition, disorder, illness, disease or symptom. For HIV, one or more symptoms or pathologies associated with HIV infection or pathogenesis include, illness such as fever, fatigue, swollen lymph nodes, a relative or progressive reduction or loss of CD4+ T cell numbers, opportunistic infections, disorders and diseases, vaccination, as set forth herein and known in the art.

For prophylactic treatment, a method is performed immediately or within 0-72 after suspected contact with, or 0-4 weeks, e.g., 1-3 days or weeks, prior to anticipated or possible exposure to, contact with, or manifestation of the physiological condition, disorder, illness, disease or symptom. For prophylactic treatment in connection with immunization/vaccination of a subject, a molecule that binds to and modulates TRAIL (Apo-2L) or TRAIL receptor (DR4 or DR5) expression or activity, such as an inhibitor (e.g., antagonist), can be administered prior to, concurrently with or following immunization/vaccination of the subject.

Doses can be based upon current existing treatment protocols, empirically determined, determined using animal disease models or optionally in human clinical studies. For example, initial study doses can be based upon animal studies, such as primates, and the amount of compound administered to achieve a prophylactic or therapeutic effect or benefit. The dose can be adjusted according to the mass of a subject, and will generally be in a range from about 25-250, 250-500, 500-1000, 1000-2500 or 2500-5000, 5000-25,000, 5000-50,000, 50,000-100,000 pg/kg; from about 0.1-1 ug/kg, 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, of subject body weight, two, three, four, or more times per hour, day, week, month or annually. Of course, doses can be more or less, as appropriate, for example, 0.00001 mg/kg of subject body weight to about 10,000.0 mg/kg of subject body weight, about 0.001 mg/kg, to about 100 mg/kg, about 0.01 mg/kg, to about 10 mg/kg, or about 0.1 mg/kg, to about 1 mg/kg of subject body weight over a given time period, e.g., 1, 2, 3, 4, 5 or more hours, days, weeks, months, years.

The invention provides, among other things, kits including a molecule that binds to and modulates TRAIL (Apo-2L) or TRAIL receptor (DR4 or DR5) expression or activity, combination compositions thereof and pharmaceutical compositions/formulations thereof, packaged into a suitable packaging material. In one embodiment, a kit includes packaging material, a molecule that binds to and modulates TRAIL (Apo-2L) or TRAIL receptor (DR4 or DR5) expression or activity, and instructions. In various aspects, the instructions are for administering a molecule that binds to and modulates TRAIL (Apo-2L) or TRAIL receptor (DR4 or DR5) expression or activity, as set forth herein for the methods embodiments, in vitro, ex vivo and in vivo.

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.). A kit can contain a plurality of components, e.g., two or more molecules that bind to and modulates TRAIL (Apo-2L) or TRAIL receptor (DR4 or DR5) expression or activity, alone or in combination with an another treatment or drug (e.g., an immune enhancing or immune suppressing drug or agent, an anti-infectious agent, an anti-cell proliferative drug or agent, an anti-HIV drug or agent), optionally sterile.

A kit optionally includes a label or insert including a description of the components (type, amounts, doses, etc.), instructions for use in vitro, in vivo, or ex vivo, and any other components therein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, hard disk, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacturer location and date, expiration dates.

Labels or inserts can include information on a physiological condition, disorder, illness, disease or symptom, for which a kit component may be used. Labels or inserts can include instructions for a clinician or subject for using one or more of the kit components in a method, treatment protocol or therapeutic/prophylactic regimen, including the methods embodiments. Instructions can include amounts of compound, frequency or duration of administration, and instructions for carrying out any of the methods, treatment protocols or prophylactic or therapeutic regimes described herein.

Labels or inserts can also include information on any desired effect or benefit, or adverse side effects, a kit component may provide, such as a prophylactic or therapeutic effect or benefit. For example, a label or insert could provide a description of reducing, decreasing, inhibiting, ameliorating or preventing onset, severity, duration, progression, frequency or probability of the physiological condition, disorder, illness, disease or symptom.

Labels or inserts can further include information on potential adverse side effects of a method embodiment. Labels or inserts can further include warnings to the clinician or subject regarding situations or conditions where a subject should stop or reduce use of a particular kit component. Adverse side effects could also occur when the subject has, will be or is currently taking one or more other medications that may be incompatible with treatment, or the subject has, will be or is currently undergoing another treatment protocol or therapeutic regimen which would be incompatible with treatment and, therefore, labels or inserts could include information regarding such side effects or incompatibilities.

Invention kits can moreover include a buffering agent, or a preservative or a stabilizing agent in a pharmaceutical formulation containing a compound of the invention. Each component of the kit can be enclosed within an individual container and all of the various containers can be within a single package. Invention kits can be designed for cold storage.

Invention kits can additionally include components, such as devices for practicing a method of the invention or administering a molecule that binds to and modulates TRAIL (Apo-2L) or TRAIL receptor (DR4 or DR5) expression or activity, to a subject, ex vivo or in vivo. The device can be a delivery device, such as a syringe, a compressible (e.g., squeezable) tube or dermal patch for mucosal, skin/dermis or corneal delivery, or an aerosol delivery device for administration to lungs or airways.

The invention provides, among other things, methods of screening for and identifying agents that modulate a T cell response mediated at least in part by TRAIL (Apo-2L). In one embodiment, a method includes contacting a T cell with a test agent; measuring a T cell response mediated at least in part by TRAIL (Apo-2L) in the presence of the test agent; and determining whether the test agent modulates the T cell response. A test agent that modulates T cell response is an agent that modulates a T cell response mediated at least in part by TRAIL (Apo-2L). Exemplary T cell responses are forth herein or known in the art and include, for example, T cell proliferation, activation-induced cell death (AICD) or apoptosis, production of TRAIL, cytotoxicity, expression or secretion of a chemokine or cytokine, or expression of a receptor that binds to a chemokine or cytokine.

The invention provides, among other things, methods of screening for and identifying agents that inhibit or prevent apoptosis of CD8+ T cells primed in the absence of CD4+ cells, wherein said apoptosis is mediated at least in part by TRAIL (Apo-2L). In one embodiment, a method includes contacting a CD8+ T cell with a test agent; measuring CD8+ T cell numbers in the presence of the test agent; and determining whether the test agent inhibits or prevents apoptosis of CD8+ T cells, which can occur during restimulation of the CD8+ T cells with an antigen. A test agent that inhibits or prevents reduction in numbers of CD8+ T cells, or increases numbers of CD8+ T cells, is an agent that inhibits or prevents apoptosis of CD8+ T cells. In various aspects, CD8+ T cell numbers are measured following or during restimulation of the CD8+ T cells with an antigen.

The invention provides, among other things, methods of identifying the presence of non-memory cells, T cells primed without CD4+ T cell help. In one embodiment, a method includes determining TRAIL (Apo-2L) expression or secretion by T cells, wherein TRAIL (Apo-2L) expression or secretion by T cells identifies the presence of non-memory cells, T cells primed without CD4+ T cell help.

The invention provides, among other things, methods of monitoring amounts of non-memory cells, T cells primed without CD4+ T cell help. In one embodiment, a method includes determining TRAIL (Apo-2L) expression or secretion by CD8+ T cells. The amount of TRAIL (Apo-2L) indicates amounts of non-memory cells, T cells primed without CD4+ T cell help.

The invention provides, among other things, methods of diagnosing a physiological disorder or disease associated with undesirable or abnormal high or low amounts of non-memory cells, T cells primed without CD4+ T cell help. In one embodiment, a method includes determining an amount of TRAIL (Apo-2L) produced (expressed or secreted) by CD8+ T cells (e.g., CD8+ T cells primed in the absence of CD4+ T cell help, antigen-specific CD8+ T cells that produce TRAIL upon antigen re-encounter). The amount of TRAIL (Apo-2L) produced indicates the presence of or predisposition towards a physiological disorder or disease associated with undesirable or abnormal high or low amounts of non-memory cells, T cells primed without CD4+ T cell help.

The invention provides, among other things, methods of screening for and identifying non-memory cells, T cells primed without CD4+ T cell help. In one embodiment, a method includes obtaining cells from a subject; contacting the cells with an antigen; and determining TRAIL (Apo-2L) expression or secretion of the cells. TRAIL (Apo-2L) expression by the cells or soluble TRAIL (Apo-2L) secreted from the cells identifies the non-memory cells.

The invention provides, among other things, methods of screening for and identifying a subject that is a candidate for TRAIL (Apo-2L) suppressive therapy. In one embodiment, a method includes providing a biological sample comprising lymphocytes from a subject; and assaying the sample for CD8+ cells that produce TRAIL (Apo-2L). The presence of CD8+ cells that produce TRAIL (Apo-2L), for example, upon antigen re-encounter with an antigen, identifies the subject as a candidate for TRAIL (Apo-2L) suppressive therapy.

The invention provides, among other things, methods of screening for and identifying a subject that is a candidate for vaccination or immunization with an antigen. In one embodiment, a method includes providing a biological sample comprising lymphocytes from a subject; and assaying the sample for the presence of CD8+ cells that produce TRAIL (Apo-2L). Absence of CD8+ cells that produce TRAIL (Apo-2L) upon antigen re-encounter identifies the subject as a candidate for vaccination or immunization.

The invention provides, among other things, methods of diagnosing a subject having a deficient immune response against an antigen. In one embodiment, a method includes providing a biological sample comprising lymphocytes from a subject; and assaying the sample for CD8+ cells specific for the antigen that produce TRAIL (Apo-2L). Detecting CD8+ cells specific for the antigen that produce TRAIL (Apo-2L) upon antigen re-encounter diagnoses the subject as having a deficient immune response against the antigen.

In various aspects, a subject is immunosuppressed, is HIV positive, has HIV antigen specific CD8+ cells that produce TRAIL (Apo-2L) upon antigen re-encounter, has reduced numbers of CD4+ cells, is suffering from a progressive reduction or loss of CD4+ cell numbers, has less than 600/cubic millimeter (mm3) blood CD4+ cells, or less than 300/cubic millimeter (mm3) blood CD4+ cells, or less than 200/cubic millimeter (mm3) blood CD4+ cells, has less than 40% CD4+ cells as a percentage of all lymphocytes in blood, or less than 25% CD4+ cells as a percentage of all lymphocytes in blood, or less than 15% CD4+ cells as a percentage of all lymphocytes in blood. In an additional aspect, the CD8+ cells producing TRAIL (Apo-2L) are antigen specific. In further aspects, an antigen comprises a bacterial, viral (e.g., HIV or hepatitis), fungal, parasite, tumor or cancer, or prion antigen.

Unless otherwise defined, all technical and scientific terms used herein have the same meaning as commonly understood by one of ordinary skill in the art to which this invention relates. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “a molecule that binds to and modulates TRAIL (Apo-2L) or TRAIL receptor (DR4 or DR5) expression or activity” includes a plurality of molecules that bind to and modulates TRAIL (Apo-2L) or TRAIL receptor (DR4 or DR5) expression or activity; and reference to “a symptom” includes a plurality of symptoms (e.g., adverse or undesirable). Of course, this does not preclude limiting certain embodiments of the invention to specific molecules that bind to and modulates TRAIL (Apo-2L) or TRAIL receptor (DR4 or DR5) expression or activity (e.g., antagonists or agonists), particular symptoms, particular conditions, disorders or diseases, particular subjects, etc., using appropriate language.

The invention is generally disclosed herein using affirmative language to describe the numerous embodiments. The invention also includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless expressly or inherently disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes various materials and methods.

Mice and cell lines: C57BL/6J, LPR/LPR, TNFR1^{-/-}, B6.SJL-Ptpcr^{a} (B6/SJL), and B6.5JL/I-Aβ^{-/-} (all H-2b) mice were purchased from The Jackson Laboratory (Bar Harbor, Me.). Bcl-2-Tg, Bcl-X_{L}-Tg, TRAIL^{-/-}″ and act-mOVA mice on a C57BL/6J background have been previously described (Cretney et al., J Immunol 168:1356 (2002); Strasser et al., Cell 67:889 (1991); Grillot et al., J Exp Med 182:1973 (1995)). The act-mOVA/K^{b-/-} variant was generated by intercrossing with the H-2K^{b-/-} strain (Pascolo et al., J Exp Med 185:2043 (1997)). Mice were maintained by in-house breeding at the La Jolla Institute for Allergy and Immunology under specific pathogen-free conditions in accordance with guidelines by the Association for Assessment and Accreditation of Laboratory Animal Care International. Mouse embryo cell lines (MEC) expressing the human adenovirus type 5 early region 1 (Ad5E1) were produced by transfection of both C57BL/6 TAP^{+/+} and TAP^{-/-} MEC lines and have been previously described (Schoenberger et al., J Immunol 161:3808 (1998)). MEC were cultured in DMEM supplemented with 10% fetal calf serum, 50 μM 2-mercaptoethanol, 2 mM L-glutamine, 20 U/mL penicillin, and 20 μg/mL streptomycin.

Immunizations and antibody treatment: Mice were immunized subcutaneously in the right flank with 1×10⁷ irradiated (3000 rad) TAP^{-/-}-Ad5E1-MEC, 2×10⁷ irradiated (1500 rad) act-mOVA/K^{b-/-}-splenocytes, or were inoculated intraperitoneally with 2×10⁵ PFU LCMV Armstrong. Depletion of CD4⁺ cells in vivo was performed by intraperitoneal administration of 150 μg GK1.5 antibody on the first three days before immunization (CD4-depleted at the time of priming) or three days after immunization (intact at time of priming) (Janssen et al., Nature 421:852 (2003)). Administration of GK1.5 antibody was continued every 3 days in all mice for the entire length of the experiments to prevent repopulation (Dialynas et al., J Immunol 131:2445 (1983)).

Isolation and stimulation of CD8⁺ T cells: CD8⁺ T cells were purified from the spleens and lymph nodes of previously immunized mice by antibody-directed complement lysis or FACS sorting after GP₃₃₋₄₁/K^{b} tetramer staining (van Stipdonk et al., Nat Immunol 2:423 (2001)). The resulting cells were >95% pure CD8⁺ T cells, and contained less than 0.1% CD4⁺ T cells as demonstrated by FACS analysis. In some expansion studies CD8⁺ T cells were CFSE labelled before use.

For E1B-specific responses purified CD8⁺ T cells from TAP^{-/-}-Ad5E1-MEC-immunized mice were stimulated in vitro for 6 days with irradiated syngeneic TAP^{+/+}-Ad5E1-MEC (10:1 ratio). CD8⁺ T cells from mice immunized with act-mOVA/K^{b-/-}-splenocytes were stimulated for 6 days in vitro at a 10:1 ratio with irradiated MEC.B7.Sig-OVA cells (Janssen et al., Nature 421:852 (2003)). CD8⁺ T cells from LCMV-infected mice were stimulated for 7 days in vitro at a 10:1 ratio with irradiated syngeneic I-Aβ^{-/-} thioglycollate induced macrophages infected with LCMV or pulsed with GP₃₃₋₄₀ peptide (KAVYNFATC; 5 μg/ml) (Janssen et al., Nature 421:852 (2003)). Transwell studies were performed in 24 well plates using polycarbonate membrane inserts (0.4 μm pore, Corning Inc., NY). qVD-OPh (quinoline-Val-Asp-CH2-difluorophenoxy) and zVAD-fmk (benzyloxycarbonyl-Val-Ala-Asp-fluoromethyl ketone) (20 μM, ICN, CA), TNFR1-Fc, Fas-Fc, TRAIL-R-Fc (all 5 μg/ml, R&D Systems, MN, USA), or N2B2 (10 μg/ml, eBioscience, CA; Kayagaki et al., J. Immuonol. 163:1906 (1999)) and rmTRAIL (Biomol, PA, USA) were added to indicated cultures. At the end of all stimulations, viable cells were collected by Ficoll gradient (Cedarlane Laboratories, Canada).

Enumeration of antigen-specific CD8⁺ T cells: Spleen and lymph node cells were incubated for 5 hours with E1B₁₉₂₋₂₀₀ peptide (VNIRNCCYI), OVA₂₅₇₋₂₆₄ (SIINFEKL), or GP₃₃₋₄₀ (KAVYNFATC) at 5 μg/ml final concentration in the presence of Brefeldin A directly ex vivo, after CD8 purification, or following in vitro culture. Surface staining for CD8 and intracellular cytokine staining for IFN-γ and TNF-α was performed using a Cytofix/Cytoperm Kit (Pharmingen, CA) according to the manufacturer's directions. The fold expansion of specific CD8⁺ T cells was calculated by dividing the absolute number of IFN-γ⁺CD8⁺ cells after in vitro culture by the absolute number of IFN-γ⁺CD8⁺ cells at the start of the culture.

Real-time reverse transcription-PCR(RT-PCR): Purified CD8⁺ T cells were stimulated with their cognate peptides for various lengths of times. CD8⁺ T cells were either used directly or Ag-specific CD8⁺ T cells were further purified by FACS sorting in combination with cytokine staining for IFN-γ or specific peptide/MHC-tetramer. Total RNA was isolated from purified CD8⁺ T cells using TriZol (Gibco BRL, Rockville, Md.) according to the manufacturer's instructions. RNA was reverse transcribed by M-MLV reverse transcriptase (Gibco BRL) using random hexamers (Gibco BRL). Sequence specific primers for murine FasL, TNF-α, TRAIL, TRAIL-R2/DR5, GFP and LacZ and 18S were previously described (Droin et al., Mol Cell Biol 23:7638 (2003), Pinkoski et al., J Biol Chem 277:42380 (2002)). Specific primers were used for murine:

Bcl-2 (forward primer: 5′-ACTTCGCAGAGATGTCCAGTCA-3′;
(SEQ ID NO: 10)
reverse primer: 5′-TGGCAAAGCGTCCCCTC-3′;
(SEQ ID NO: 11))
Bcl-XL (forward primer: 5′-TCGGGATGGAGTAAACTGGG-3′;
(SEQ ID NO: 12)
reverse primer: 5′-CCACGCACAGTGCCCC-3′;
(SEQ ID NO: 13))
FLIP (forward primer: 5′-AGCAACCGTGGAGGACCA-3′;
(SEQ ID NO: 14)
reverse primer: 5′-CCATCAGCAGGACCCTATAATCA-3′;
(SEQ ID NO: 15))
and
L32 (forward primer: 5′-GAAACTGGCGGAAACCCA-3′;
(SEQ ID NO: 16)
reverse primer: 5′-GGATCTGGCCCTTGAACCTT-3′.
(SEQ ID NO: 17))

Real Time PCR™ was performed with AmpliTaq Gold™ polymerase in a PE Biosystems 5700 thermocycler using SyBr Green™ detection protocol as outlined by the manufacturer. Briefly, 12 ng of total cDNA, 50 nM of each primer and 1× SyBr Green mix were used in a total volume of 25 μl. L32 and 18S were used as internal controls.

Transient transfection and reporter assays: In transient expression experiments, all transfections were performed using Superfect (Qiagen, Valencia, Calif.) according to the manufacturer's instructions with 0.2 μg of each vector. 1.5×10⁶ helped or helpless CD8⁺ T cells were transfected and incubated 13 hours at 37° C. before stimulation in vitro with E1B₁₉₂₋₂₀₀ or GP₃₃₋₄₁ peptides. Due to the low efficiency of transient transfection, GFP mRNA was analyzed by real time PCR and transfection efficiencies were measured by analysis of LacZ reporter mRNA expression.

Human TRAIL promoter construct (397 bp) was generated by PCR using the 5′-CGACGCGTCCACATATGGAAGTTTCAGGTC-3′ (SEQ ID NO:18) and the 5′-GGAAGATCTTGAAAGCGAATGAGTTGTTTTTCTGGG-3′ (SEQ ID NO:19) primers. Amplified fragment was cloned directly into pGLOW vector (Invitrogen, Carlsbad, Calif.) and sequenced according to the manufacturer's instructions (Drion et al., Mol Cell Biol 23:7638 (2003)).

Subjects and Sample Collection: Peripheral blood was obtained from 29 HIV-positive (2 women, 27 men, ages between 39 and 75 years) from the Special Immunology Unit at University Hospitals (Cleveland, Ohio). The CD4⁺ T cell counts ranged from 4 to 1232 cells/mm³. The patients were categorized according to their CD4⁺ T cell counts into two groups: CD4⁺<200 (HIV⁺CD4^{low}; n=21) and CD4⁺>200 (HIV⁺CD4^{high}; n=8). The 22 healthy controls analyzed (HIV⁻CD4^{high}) (9 women, 13 men, ages between 21 and 64) were health care workers or members of our laboratory and adjoining laboratories. PBMC were isolated from 40 to 100 ml of heparinized blood by standard Ficoll density-gradient centrifugation (Isoprep, Robbins Scientific, Sunnyvale, Calif.). All studies were performed under the approval of the Institutional Review Board for Human Investigation at the University Hospitals of Cleveland.

ELISPOT assay: Depending on the cell count of the subjects, ImmunoSpot plates (Cellular Technology Limited, Cleveland, Ohio) or UNIFILTER small-volume plates (Whatman, New Jersey, NY) were coated with capture antibody diluted in PBS at 4° C. overnight. For capturing IFN-γ mAb M700A from Endogen (Woburn, Mass.) was used at a concentration of 5 μg/ml, for capturing TRAIL polyclonal rabbit anti-mouse Ab, cross-reactive to human, (abcam; Cambridge, Mass.) was used at a concentration of 1 μg/ml. The plates were then blocked with PBS containing 1% BSA for 1 hour and washed three times with PBS. Cells were plated in serum-free medium obtained from Cellular Technology Limited. When ImmunoSpot plates were used 2-3·10⁵ cells were plated per well, for the small-volume plates 1.4·10⁵ cells were plated. All results were normalized to 3·10⁵ cells. CD4-, CD8- or CD3-depleted cells as well as CD8⁺-enriched cell populations were obtained by negative selection using depletion or enrichment cocktails (RosetteSep™; StemCell Technologies, Vancouver, British Columbia, Canada) and standard Ficoll-density gradient centrifugation. Antigens used were HIV-1 gag 20mer peptides (10 μg/ml) comprising the HIV-1 subtype B region, with 10-amino acid overlaps between sequential peptides and HIV-1 gag, env, pol and nef 15mer peptides with 11-amino acid overlaps between sequential peptides (10 g/ml). As a control CEF peptides (2 μg/ml) were used (Currier et al., J Immunol Methods 260:157 (2002)). These peptides cover HLA class I restricted determinants of viruses that commonly infect humans, in particular Cytomegalovirus (CMV), Epstein-Barr Virus (EBV) and Influenza (Flu). HCV 18mer peptides (10 μg/ml) were prepared by Mimotopes. All other peptides listed above were obtained from the NIH AIDS Research & Reference Reagent Program, Division of AIDS, NIAID, NIH: CEF Control Peptide pool from DADS, NIAID. Phytohemagglutinin (PHA) was obtained from Sigma (St. Louis, Mo.; 10 μg/ml). Control wells contained PBMC with medium alone. After 24 hours of incubation at 37° C. and 7% CO₂ the plates were washed. For IFN-γ biotinylated detection mAb M701 (Endogen, 1 μg/ml) was added, for TRAIL purified detection mAb 75411.11 (Sigma; St. Louis, Mo.; 2 μg/ml). The plates were then incubated at 4° C. overnight. All antibodies were diluted in PBS containing 1% BSA and 0.025% TWEEN (Fisher Scientific International Inc., Hampton, N.H.). Subsequently, Streptavidin-AP (DakoCytomation, Glostrup, Denmark) diluted in PBS/BSA/TWEEN at 1:1000 was added to the IFN-γ plates for 1 hour at room temperature. These plates were developed the same day by using nitroblue tetrazolium/5-bromo-4-chloro-3-indolyl phosphatase substrate (Kirkegaard & Perry Laboratories, Gaitherburg, Md.) for 10 min. For the TRAIL-ELISPOT anti-mouse IgGAM (H+L) (Invitrogen, Carlsbad, Calif.) was added and the plates were incubated overnight at 4° C. The next day these plates were developed as described above. The plates were air dried overnight before conducting image analysis using an ImmunoSpot Series 3B Analyzer (Cellular Technology Limited).

Flow cytometric analysis: Flow cytometry was performed as described previously (Tary-Lehmann et al., J Exp Med 175:503 (1992)) using a Becton Dickinson FACSan and staining with FITC-labelled anti-CD8 or anti-CD3 (purified and labelled, clones OKT8 and OKT3) and PE-labelled anti-CD4 antibodies (BD-Pharmingen). Ten thousand live cells were analyzed per sample.

Statistical analysis: Mann-Whitney Rank Sum test was calculated by SigmaStat (version7.0, SPSS, Chicago, Ill.) to evaluate the statistical significance of TRAIL secretion among the different subject groups. Statistical significance was set at P<=0.05.

### Example 2

This example describes studies of CD8+ T cell proliferative response upon antigen restimulation.

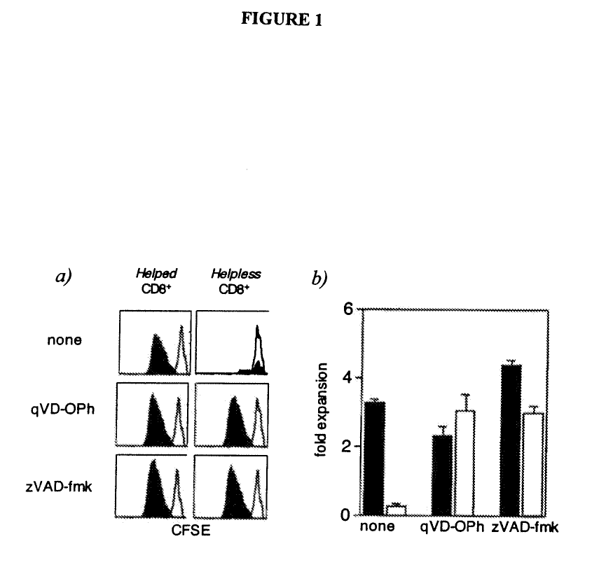

As a first step towards identifying the defect present in helpless CD8⁺ T cells, their capacity to initiate a secondary proliferative response upon restimulation was studied. This was done using an in vivo cross-priming system that features immunization with a cellular vaccine expressing an H-2D^{b}-restricted model antigen, E1B₁₉₂₋₂₀₀ (TAP^{-/-} Ad5E1-MEC) (Schoenberger et al., J Immunol 161:3808 (1998)). Seven days after in vivo priming with 10⁷ irradiated Tap^{-/-}Ad5E1-MEC, CD8⁺ T cells were obtained and labelled with the viable fluorescent dye CFSE (5,6-carboxy-succinimidyl-fluorescein-ester) to allow visualization of the clonal expansion (Lyons et al., J Immunol Methods 171:131 (1994)). CFSE dilution was assessed in E1B₁₉₂₋₂₀₀-specific IFN-γ-producing effector CD8⁺ T cells ex vivo and 4 days after in vitro restimulation on TAP^{+/+}Ad5E1-MEC⁵ in the presence of caspase inhibitors qVD-OPH and zVAD-fmk. Effector CD8⁺ T cells from control mice (helped CD8⁺ T cells) both proliferated and accumulated upon secondary encounter with antigen, with CFSE dilution profiles indicating that they underwent multiple rounds of division (FIG. 1a, top left panel). The majority of CD8⁺ T cells obtained from CD4-depleted mice (helpless CD8⁺ T cells), in contrast, either failed to divide or went through a limited number (1 or 2 rounds) of divisions (FIG. 1a, top right panel). The absolute number of E1B₁₉₂₋₂₀₀-specific cells in these cultures decreased, suggesting that these helpless cells were dying following restimulation.

The secondary expansion of IFN-γ-producing E1B₁₉₂₋₂₀₀-specific CD8⁺ T cells from intact (helped) and CD4-depleted (helpless) mice immunized 7 days earlier with 10⁷ irradiated Tap^{-/-}Ad5E1-MEC was determined by intracellular IFN-γ staining 6 days after in vitro restimulation with Tap^{+/+} Ad5E1-MEC in the presence or absence of the indicated caspase inhibitors. Addition of caspase inhibitors quinoline-Val-Asp-CH2-difluorophenoxy (qVD-OPh) and benzyloxycarbonyl-Val-Ala-Asp-fluoromethyl ketone (zVAD-fmk) restored secondary expansion in the purified helpless CD8⁺ T cells, and resulted in comparable proliferation and accumulation to that observed in the helped CD8⁺ T cells (FIGS. 1a and 1b).

These results indicate that helpless CD8⁺ T cells have the potential to proliferate and survive upon secondary stimulation, but this outcome may be blocked or suppressed by the induction of caspase-mediated activation-induced cell death (AICD).

### Example 3

This examples describes data indicating that helpless CD8⁺ T cells selectively up-regulated mRNA for TNF and TRAIL receptor DR5 (TRAIL-R2) following antigen restimulation.

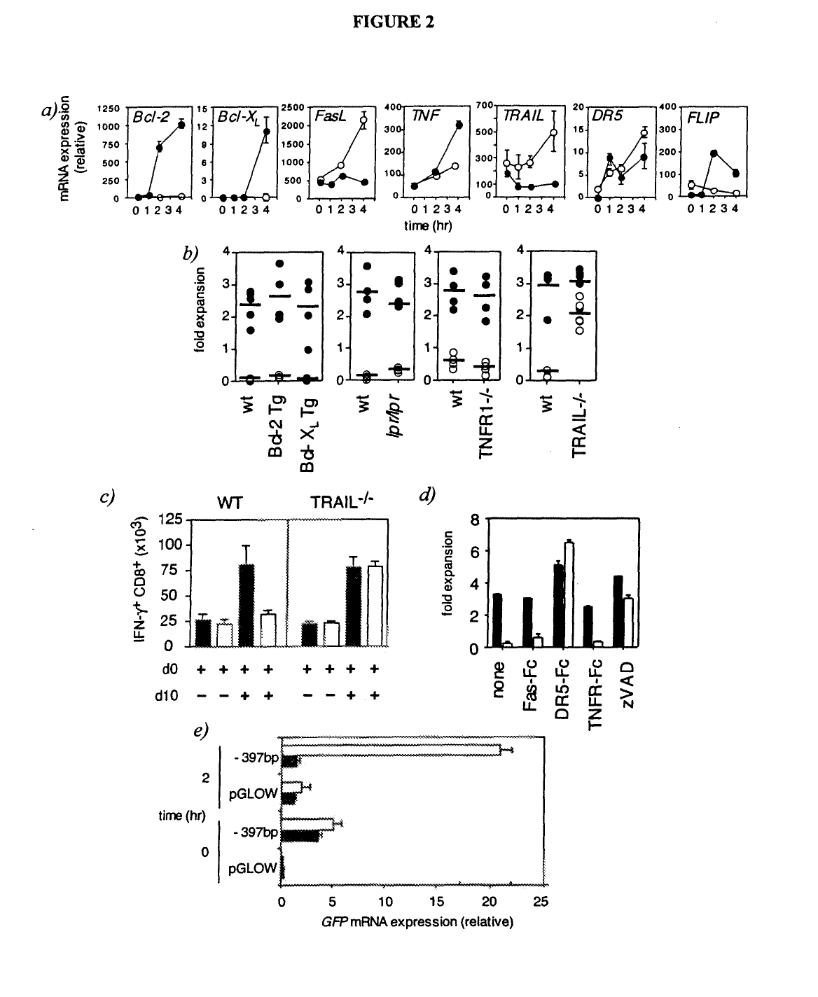

To gain insight into the molecular pathways underlying the disparate secondary responses of helped and helpless CD8⁺ T cells, a set of apoptosis-related candidate genes was analysed in the restimulated CD8⁺ T cells by real-time RT-PCR. mRNA levels for the apoptosis-related genes Bcl-2, Bcl-xL, FasL, TNF-α, TRAIL, DR5, and c-FLIP were determined by real-time RT-PCR at different time points following in vitro E1B₁₉₂₋₂₀₀-peptide restimulation of purified CD8⁺ T cells obtained from intact and CD4-depleted mice immunized 7 days earlier with 10⁷ irradiated Tap^{-/-}Ad5E1-MEC. Notably, mRNAs for Bcl-2, Bcl-xL and c-FLIP, encoding anti-apoptotic proteins, were selectively up-regulated upon activation of helped CD8⁺ T cells (FIG. 2a), cells that subsequently expanded and accumulated. In contrast, mRNAs for FasL and TNF-related apoptosis-inducing ligand (TRAIL) (also know as Apo2-ligand or Apo-2L) (Pitti et al., J Biol Chem 271:12687 (1996); Wiley et al., Immunity 3:673 (1995)), both of which encode pro-apoptotic proteins, were selectively up-regulated in helpless CD8⁺ T cells (FIG. 2a), cells which subsequently failed to expand. These results indicate that both purified helped and helpless CD8⁺ T cells up-regulated mRNA for TNF and TRAIL receptor DR5 (TRAIL-R2) (Walczak et al., Embo J 16:5386 (1997); Pan et al., Science 277:815 (1997); MacFarlane et al., J Biol Chem 272:25417 (1997)) following restimulation (FIG. 2a).

### Example 4

This example shows data demonstrating that although CD8⁺ T cells primed in the presence or absence of T help undergo primary expansion with comparable kinetics (Janssen et al., Nature 421:852 (2003)), their progeny undergo fundamentally different programs of gene expression upon secondary antigen stimulation.

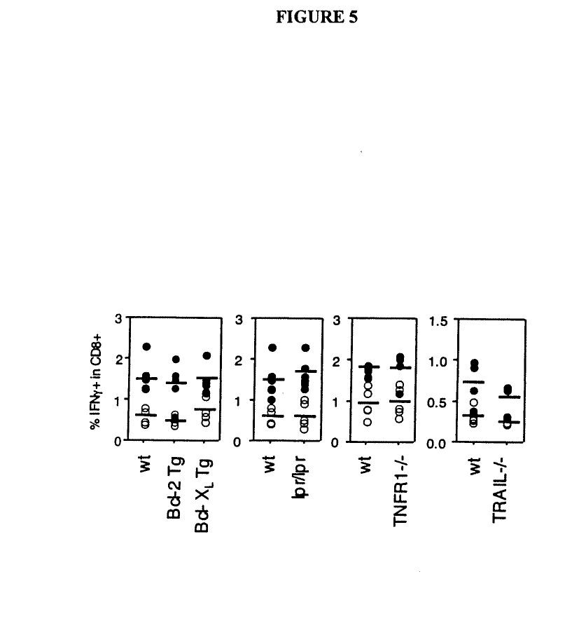

To further delineate the contribution of the up-regulated genes, primary and secondary CD8⁺ T cell responses were studied in a set of genetically-manipulated mice in which Bcl-2 and Bcl-xL are constitutively expressed, or in which TNFR, Fas, and TRAIL expression is absent. Intact and CD4-depleted mice from certain strains were immunized with Tap^{-/-}Ad5E1-MEC. Seven days later CD8⁺ T cells were purified and the frequency of E1B₁₉₂₋₂₀₀-specific CD8⁺ T cells was determined by intracellular IFN-γ stain upon stimulation with E1B₁₉₂₋₂₀₀ peptide. The frequency of E1B₁₉₂₋₂₀₀-specific CD8⁺ T cells in control and CD4-depleted mice was determined directly ex vivo (see FIG. 5 for enumeration of primary IFN-γ⁺CD8⁺effectors) and their capacity for secondary expansion was assessed following antigen restimulation in vitro.

Intact and CD4-depleted mice from certain strains were immunized with Tap^{-/-} Ad5E1-MEC. The frequency of IFN-γ-producing E1B-specific CD8⁺ T cells was determined directly ex vivo and again following a 6 day coculture with Tap^{+/+}Ad5E1-MEC. The fold expansion of IFN-γ producing E1B₁₉₂₋₂₀₀-specific CD8⁺ T cells was calculated as the increase in the absolute number of specific CD8⁺ T cells. In each of the mouse strains analyzed, helped CD8⁺ T cells underwent a similar degree of secondary expansion upon restimulation (FIG. 2b). However, neither enforced expression of Bcl-2 or Bcl-xL nor the absence of Fas (lpr/lpr) or TNFR (TNFR1^{-/-}) rescued secondary expansion in helpless CD8⁺ T cells (FIG. 2b). Effector T cells from TRAIL-deficient mice, in contrast, did not display the helpless phenotype when primed in the absence of CD4⁺ T cells, and instead underwent substantial secondary expansion following restimulation (FIG. 2b).

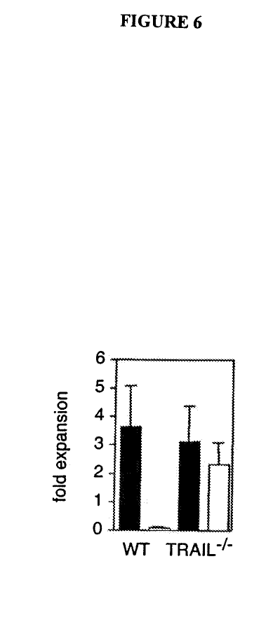

This result was also observed using a second cross-priming system in which CD8⁺ T cells specific for a different antigen were enumerated physically using peptide/MHC tetramers (FIG. 6). Intact and CD4-depleted mice from certain strains were immunized with 2×10⁷ irradiated act-mOVA/K^{b-/-}-splenocytes. Seven days later, splenocytes were stimulated with irradiated MEC.B7.Sig-OVA cells for 6 days in vitro. The expansion of OVA₂₅₇₋₂₆₄-specific CD8⁺ T cells was calculated as the fold-increase in the absolute number of specific CD8⁺ T cells. In this system, mice were immunized with splenocytes from act-mOVA/K^{b-/-} mice that express a membrane-bound form of the model antigen chicken ovalbumin (OVA), but which lack expression of the H-2K^{b} MHC class I molecule required for presentation of the immunodominant OVA epitope (OVA₂₅₇₋₂₆₄) to CD8⁺ T cells (Ehst et al., Am J Transplant 3:1355 (2003)). Taken together, these results indicate that secondary expansion of helpless CD8⁺ T cells is constrained by TRAIL.

To determine whether TRAIL exerted a similar effect in vivo, intact and CD4-depleted wild type and TRAIL^{-/-} mice were immunized with 2×10⁷ irradiated act-mOVA/K^{b-/-} splenocytes (day 0), with some groups challenged again with act-mOVA/K^{b-/-} splenocytes 10 days later. The absolute number of OVA₂₅₇₋₂₆₄-specific CD8⁺ T cells per spleen was determined 4 days later by intracellular IFN-γ staining. In this prime/boost setting, the helpless OVA₂₅₇₋₂₆₄-specific CD8⁺ T cells in TRAIL^{-/-} mice underwent secondary expansion whereas those from wild type mice did not (FIG. 2c). These results reveal that regardless of whether restimulation occurs in vitro or in vivo, helpless CD8⁺ T cells lacking TRAIL expression undergo secondary expansion.

Various TRAIL receptors have been identified, two of which are human, denoted R1or DR4 and R2 or DR5. These two receptors have been reported to have death domains within the intracellular regions. TRAIL-R1 transcript has been detected in many human tissues including spleen, peripheral blood leukocytes, small intestine and thymus. TRAIL-R2 transcript has also been detected in many human tissues including spleen, peripheral blood leukocytes and ovary.

The role of TRAIL was further studied using a soluble form of the TRAIL receptor DR5 (Dr5-Fc) to block the action of TRAIL during secondary expansion. Purified CD8⁺ T cells from Tap^{-/-}Ad5E1-MEC immunized intact (black bars) and CD4-depleted mice (white bars) were cultured with Tap^{+/+}Ad5E1-MEC in the presence or absence of the indicated soluble death receptors. After 6 days the fold expansion of IFN-γ producing E1B₁₉₂₋₂₀₀-specific CD8⁺ T cells was calculated as the increase in the absolute number of specific CD8+ T cells. The addition of DR5-Fc restored secondary expansion of helpless CD8⁺ T cells from wild-type mice (FIG. 2d). Addition of the control fusion proteins Fas-Fc or TNFR-Fc, in contrast, had no effect and none of the soluble receptors affected the secondary expansion of purified helped CD8⁺ T cells (FIG. 2d). Taken together, these results indicate the autocrine TRAIL expression prevents the secondary expansion of helpless CD8⁺ T cells.

Helpless, but not helped, CD8 + T cells express TRAIL mRNA upon restimulation (FIG. 2a), raising the possibility that TRAIL is differentially regulated at the transcriptional level in these populations. To study this, a 0.4 kB 5′-flanking sequence of the TRAIL gene was used to drive expression of a reporter in primary cells (Droin et al., Mol Cell Biol 23:7638 (2003)). Purified CD8⁺ T cells from Tap^{-/-}Ad5E1-MEC-immunized intact and CD4-depleted mice were cotransfected with empty pGLOW or pGLOW containing the bp −397 of human TRAIL promoter in presence of the β-galactosidase-expressing vector. Transfected cells were incubated 13 hours before stimulation with E1B₁₉₂₋₂₀₀ peptide. GFP mRNA was analyzed by real-time RT PCR and normalized to 18S and to LacZ mRNA (as an efficiency control) expression. Following restimulation, real-time RT-PCR was used to detect reporter expression in the rare antigen-specific transfected CD8+ T cells (FIG. 2e). Secondary stimulation led to a strong induction of the TRAIL promoter reporter in helpless CD8⁺ T cells while no induction was observed in the helped CD8⁺ T cells (FIG. 2e), indicating that the observed increase of TRAIL mRNA in helpless CD8⁺ T cells is transcriptionally regulated.

Taken together, these results demonstrate that although CD8⁺ T cells primed in the presence or absence of T help undergo primary expansion with comparable kinetics (Janssen et al., Nature 421:852 (2003)), their progeny undergo fundamentally different programs of gene expression upon secondary antigen stimulation. Of these genes, TRAIL expression is the major determinant of the outcome of secondary encounter with antigen.

### Example 5

This example includes studies indicating that helped and helpless CD8⁺ T cells are equally sensitive to TRAIL.

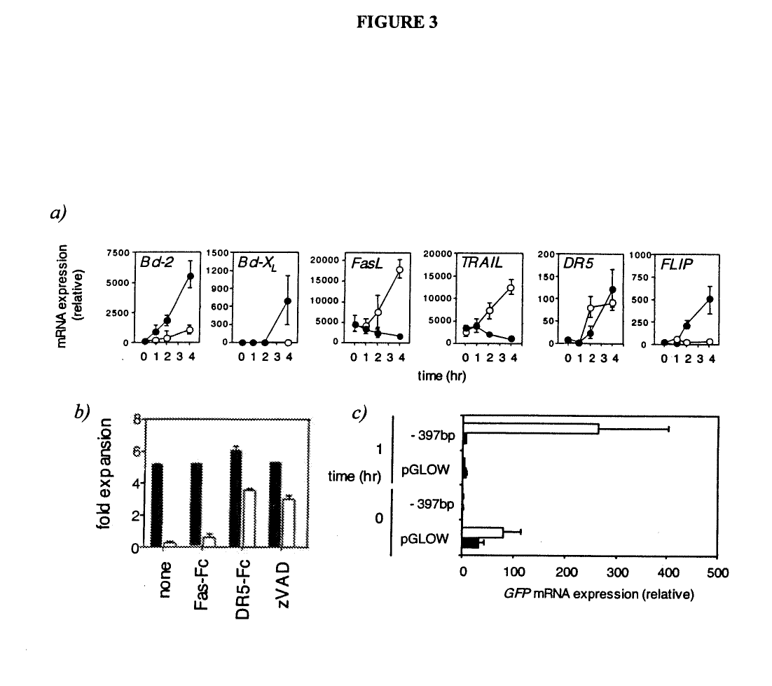

The role of TRAIL in the CD8⁺ T cell response to infection was studied using lymphochoriomeningitis virus (LCMV), a well-characterized model of viral infection. As in the cross-priming model, CD8⁺ T cells primed in the absence of T help expand and acquire effector function, but are defective in their secondary expansion (Janssen et al., Nature 421:852 (2003)). mRNA levels for Bcl-2, Bcl-xL, FasL, TNF, TRAIL, DR5, and c-FLIP were determined determined by real-time RT-PCR at different time points following in vitro GP₃₃₋₄₁-peptide restimulation of purified CD8⁺ T cells obtained from intact and CD4-depleted mice immunized 28 days earlier with 2×10⁵ PFU LCMV (Armstrong). Following antigen restimulation, LCMV-specific helped CD8⁺ T cells upregulated mRNA for anti-apoptotic genes including Bcl-2, Bcl-xL and FLIP, but not TRAIL mRNA, while the helpless CD8⁺ T cells failed to upregulate these anti-apoptotic genes and induced the expression of TRAIL mRNA (FIG. 3a).

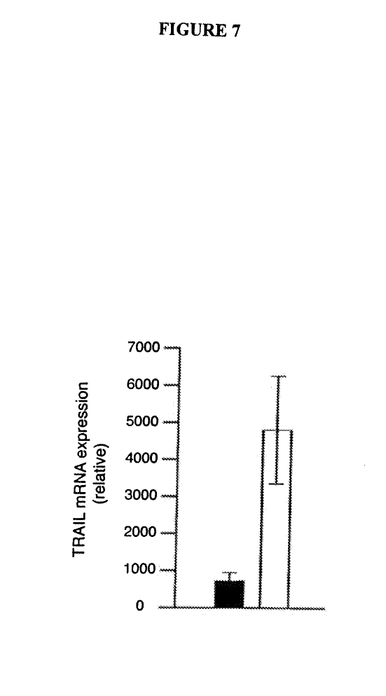

Intact and CD4-depleted mice were immunized with 2×10⁵ PFU LCMV (Armstrong). Fourteen days later, CD8⁺ T cells were purified by flow cytometry after staining with GP₃₃₋₄₁/K^{b} tetramers and the mRNA level for TRAIL was determined by real-time RT-PCR. Selective induction of TRAIL expression was also observed when helped and helpless GP₃₃₋₄₁-specific CD8⁺ T cells were isolated using GP₃₃₋₄₁/K^{b} tetramers (FIG. 7). Expression of DR5 mRNA, however, was induced in both helped and helpless CD8⁺ T cell populations (FIG. 3a).

Purified CD8⁺ T cells from immunized intact and CD4-depleted mice were cultured with LCMV-infected thioglycollate-induced macrophages in the presence or absence of soluble death receptors. After 6 days the fold expansion of was calculated as the increase in the absolute number of GP₃₃₋₄₁-specific CD8⁺ T cells. Secondary expansion of the helpless CD8⁺ T cells was restored when DR5-Fc or zVAD-fmk were added to the cultures, but not in the presence of Fas-Fc (FIG. 3b).

Purified CD8⁺ T cells from intact and CD4-depleted mice infected 28 days earlier were cotransfected with empty pGLOW or pGLOW containing the bp −397 of human TRAIL promoter in presence of the β-galactosidase-expressing vector. Transfected cells were incubated 13 hours before stimulation with GP₃₃₋₄₁ peptide. GFP mRNA was analyzed by real-time RT PCR and normalized to 18S and to LacZ mRNA (as an efficiency control) expression. The TRAIL promoter reporter was selectively induced in the restimulated helpless CD8⁺ T cells (FIG. 3c).

These data indicate that in both the cross-priming and viral infection models, TRAIL regulates secondary expansion of CD8⁺ T cells.

### Example 6

This example includes studies indicating that the TRAIL produced by helpless CD8⁺ T cells is soluble, and can therefore potentially act on cells (e.g., other lymphocytes) that are not in direct contact with the helpless CD8⁺ T cells.

Sensitivity to TRAIL-mediated apoptosis is regulated at different levels, including expression of receptor and signalling components, antagonists of receptor signalling, and differences in expression or activation of caspases and their inhibitors (Bhardwaj et al., J Clin Immunol 23:317 (2003)). In both the cross-priming and LCMV models, helpless and helped CD8⁺ T cells expressed DR5 following secondary antigenic stimulation (FIGS. 2, 3), but only the helped CD8⁺ T cells upregulated c-FLIP mRNA, encoding a protein that can potentially inhibit TRAIL mediated apoptosis (Aggarwal, Nat Rev Immunol 3:745 (2003)).

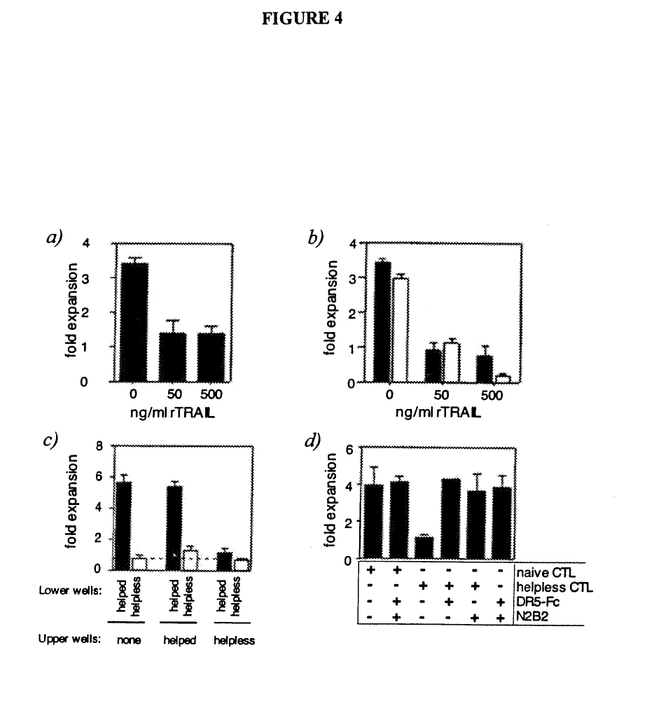

To determine if resistance to TRAIL receptor signalling contributes to secondary expansion of helped CD8⁺ T cells, effects of exogenous TRAIL in vitro was studied. Purified CD8+ T cells from intact wild type mice and intact and CD4-depleted TRAIL^{-/-} mice were cultured with Tap^{+/+} Ad5E1-MEC in the presence of absence of rmTRAIL for 6 days and the fold expansion of E1B₁₉₂₋₂₀₀-specific CD8⁺ T cells was determined. Addition of recombinant TRAIL to helped CD8⁺ T cell cultures completely inhibited secondary expansion, demonstrating that helped CD8⁺ T cells were sensitive to TRAIL-mediated effects (FIG. 4a). Helpless CD8⁺ T cells from TRAIL-deficient mice, which underwent secondary expansion (FIG. 4b), failed to do so in the presence of recombinant TRAIL.

These results indicate that helped and helpless CD8⁺ T cells are equally sensitive to the effects of TRAIL, and that the differences in their secondary response to antigen are therefore likely to be a consequence of TRAIL expression.

### Example 7

This example includes data indicating that the lack of response of helpless CD8⁺ T cells to secondary stimulation dominates over that of helped CD8⁺ T cells.

Purified helped and helpless CD8⁺ T cells were cultured with Tap^{+/+}Ad5E1-MEC in the bottom of transwell plates. In the top chamber purified helped or helpless CD8⁺ T cells were cultured with Tap^{+/+}Ad5E1-MEC. TRAIL blocking antibody or soluble DR5-Fc were added for the entire duration of culture. After 6 days the fold expansion of E1B₁₉₂₋₂₀₀-specific CD8⁺ T cells in the bottom chamber was determined as the increase in the absolute number of E1B₁₉₂₋₂₀₀-specific CD8⁺ T cells. The secondary proliferative response of each type in the lower wells was determined for helped and helpless CD8⁺ T cells (FIG. 4c). While addition of helped CD8⁺ T cells to the upper wells had no effect on the responses of either helped or helpless CD8⁺ T cells in the lower wells, the helpless CD8⁺ T cells effectively inhibited the programmed secondary expansion of the helped CD8⁺ T cells plated in the lower wells. This inhibitory effect was blocked by addition of either DR5-Fc or a neutralizing anti-TRAIL monoclonal antibody to the cultures (FIG. 4d). These studies indicate that the TRAIL produced by helpless CD8⁺ T cells is soluble, and can therefore potentially act on cells not in direct contact with the helpless CD8⁺ T cells.

The results herein shed new light on the physiological role of TRAIL in immune homeostasis. It has been previously reported that TRAIL can induce apoptotic death in a variety of transformed cells while sparing normal cells, leading to an interest in the clinical use of TRAIL for cancer immunotherapy (Pitti et al., J Biol Chem 271:12687 (1996), Smyth et al., Immunity 18:1 (2003)). Of relevance to immune function, TRAIL has been shown to contribute to activation-induced apoptosis of the human leukemia line Jurkat, in vitro activated human CD4⁺ T cell clones, and in peripheral blood lymphocytes (Zhang et al., Cell Death Differ 10:203 (2003), Martinez-Lorenzo et al., Eur J Immunol 28:2714 (1998)).

Here, it is demonstrated that TRAIL can regulate peripheral CD8⁺ T cell responses through CD4⁺ T help, a finding that may be relevant to the establishment and maintenance of peripheral tolerance. Like helpless CD8⁺ T cells, many potentially-autoreactive T cells in the periphery will encounter their cognate antigen on a non-professional or immature APC lacking sufficient costimulatory capacity, thereby potentially inducing a helpless phenotype. Re-encounter with self-antigen would then result in clonal deletion via TRAIL-mediated activation-induced cell death (AICD), thereby purging the peripheral repertoire of self-reactive clones. The transwell experiments (FIG. 4c, and d) indicate that the TRAIL produced by helpless CD8⁺ T cells is soluble and can therefore potentially act on other cells not in direct contact. During a normal response it is possible that not all CD8⁺ T cells receive the help necessary to develop into memory cells, and the emerging population would be a mix of helped and helpless cells. The capacity of helpless CD8⁺ T cells to secrete TRAIL and thereby suppress other cells in their proximity upon re-encounter with antigen may represent a novel mechanism of immune regulation.

In sum, the studies herein use a range of antigenic challenges revealing that the presence or absence of CD4⁺ T help is functionally imprinted in CD8⁺ T cells at an early time point during their clonal activation as they transit from the naïve to the primed state. Depending on the availability of help, CD8⁺ T cells will acquire distinct genetic programs that each begins with proliferation and effector differentiation, but which specify fundamentally different fates for their clonal progeny upon restimulation (AICD versus secondary expansion). The differential regulation of TRAIL expression at this later time point clearly represents a key feature of this mechanism and can account for the observed role of CD4⁺ T cells in the generation of CD8⁺ T cell memory. Helpless CD8⁺ T cells are not irretrievably consigned to death by TRAIL-mediated AICD upon restimulation, however, as cytokines such as IL-2 can permit their re-expansion when provided during secondary antigenic encounter (Janssen et al., Nature 421:852 (2003)). It is possible that other cytokines may mediate similar effects on the survival of helpless cells (Sun et al., Nat Immunol 5:927 (2004)). The signals through which the molecular signature of T help is transmitted to CD8⁺ T cells during their priming and the mechanism through which these are integrated in secondary responses remain intriguing questions.

### Example 8

This example describes studies indicating the presence of CD8⁺ T cell lethargy in HIV⁺CD4^{low} subjects.

Peripheral blood mononuclear cells (PBMC) was obtained from healthy, non-HIV-infected (n=22) and HIV-infected individuals (n=29). The latter were subdivided into two groups that either displayed less than 200 CD4⁺ T cells/mm³, (HIV⁺CD4^{low}, n=21) or more than 200 CD4⁺ T cells/mm³ (HIV⁺CD4^{high}, n=8). Subsequently, freshly isolated PBMC were analyzed without additional pre-treatment in 24 hour TRAIL or IFN-γ ELISPOT assays for HIV peptide-specific recall responses.

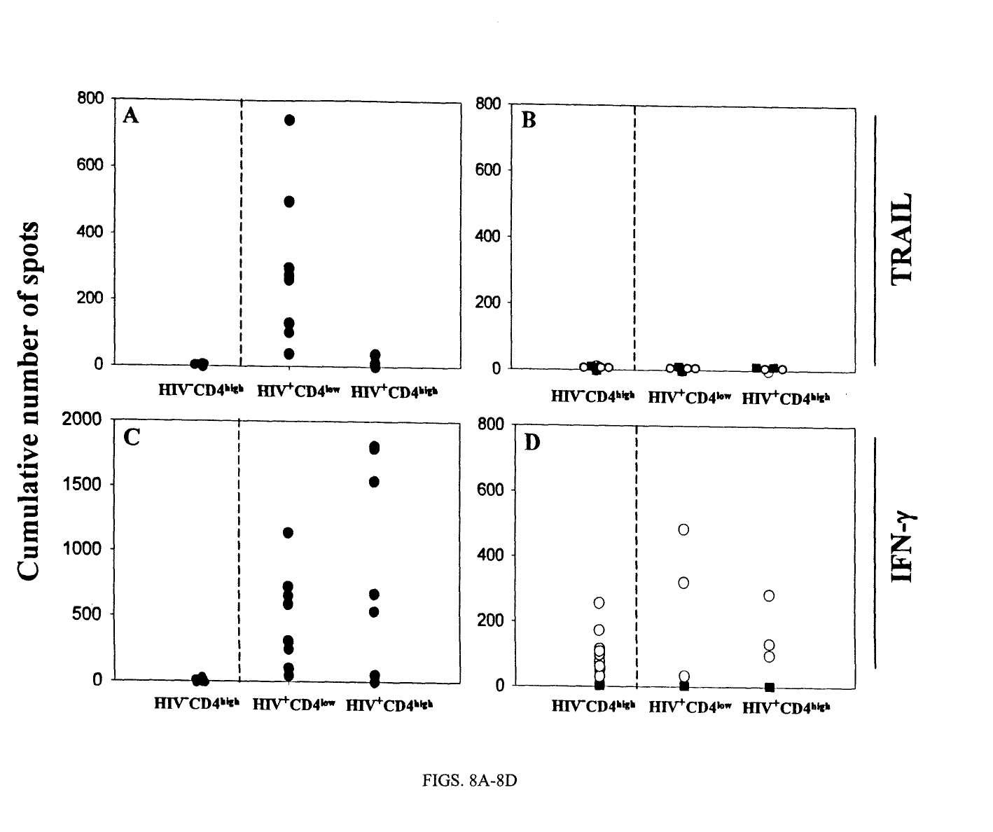

The HIV peptide library included 49 gag 20mers as well as four env, four pol and six nef peptides, the latter 15 amino acids long. These peptides were analyzed as individual peptides and in duplicate wells. A library of 16 hepatitis C virus (HCV) peptides (18mers) was used as negative control. All HIV-infected and healthy subjects were seronegative for hepatitis C. A CEF peptide library containing 23 peptides comprising sequences of Cytomegalo (CMV)—Epstein-Barr (EBV)—and Influenza (Flu) virus was employed as additional control. The results are summarized in FIG. 8.

The HIV peptides induced TRAIL-producing cells in all HIV⁺CD4^{low} donors, but in none of the HIV⁺CD4^{high} subjects (P=<0.001). Neither CEF- nor HCV peptides triggered TRAIL production in either of the HIV-infected subject groups or in any of the healthy controls. The HIV peptides did not induce TRAIL (P=<0.001) or IFN-γ (P=<0.001) in the healthy controls, but triggered IFN-γ producing cells in the HIV-infected individuals. The frequencies of HIV peptide-specific IFN-γ-secreting CD8⁺ T cells were diminished in HIV⁺CD4^{low} subjects relative to those subjects harbouring CD4⁺ T cells in higher numbers (FIG. 8), but did not reach statistical significance (P=0.170).

From the number of HIV-infected individuals analyzed 200 CD4⁺ T cells/mm³ appeared to be the cut-off value for a functional CD4⁺ T cell compartment in HIV infection. All HIV⁺CD4^{low} donors (n=21) showed TRAIL release, while none of the eight subjects with more than 200 CD4⁺ T cells/mm³ (CD4^{high}) produced TRAIL (P=<0.001). Moreover, TRAIL production was not observed in CD8⁺ T cell recall responses to CEF peptides in HIV⁺CD4^{high} subjects, or HIV⁻CD4^{high} controls. TRAIL production was antigen-specific: it was not induced by HCV peptides in HCV negative donors, or even by the CEF peptides in HIV⁺CD4^{low} subjects (FIG. 8b). This fits the notion established in murine models suggesting that the helped or helpless phenotype of CD8⁺ memory T cells is functionally imprinted during the actual priming process, (during the transition of the CD8⁺ T cells from the naïve to the memory/effector state), and subsequently becomes a stable feature of these memory CD8⁺ T cells (Janssen et al., Nature 434:88 (2005)). Infections with CMV, EBV and influenza in HIV-infected subjects have most likely occurred early in life, predating HIV infection.

Thus, the data indicates that the CEF-peptide-reactive CD8⁺ T cells in these individuals have been generated in an unimpaired CD4⁺ T cell environment, and consequently display the helped phenotype, that remains unaffected by subsequent CD4⁺ T cell loss.

### Example 9

This example describes data indicating that HIV peptide-induced TRAIL response is mediated by CD8⁺ T cells.

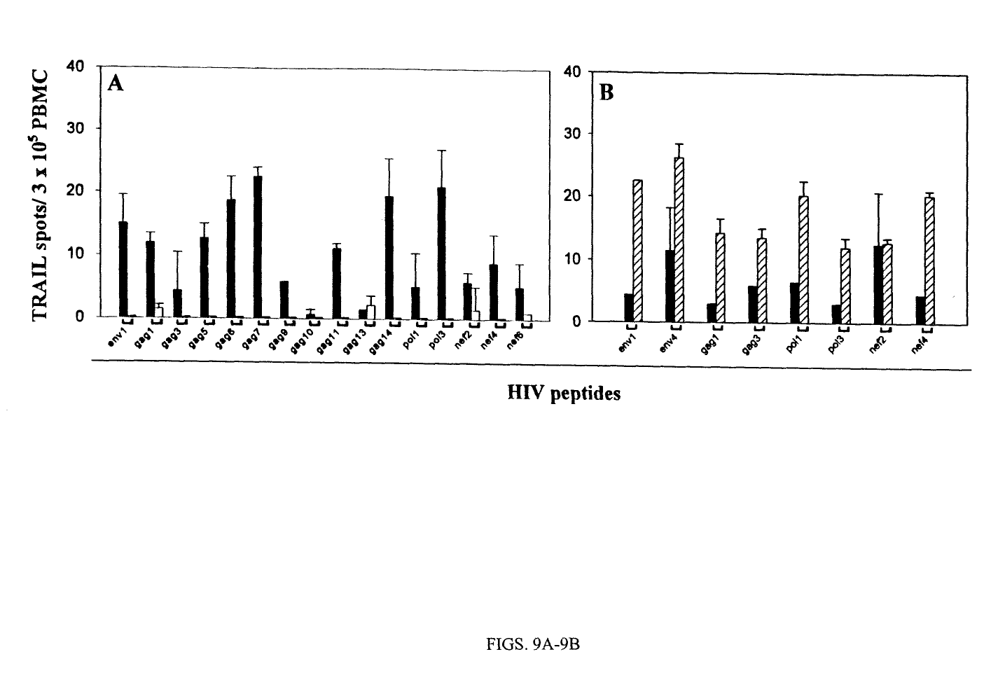

Two independent approaches were employed to define the phenotype of the TRAIL-producing cells in response to HIV peptide stimulation in HIV⁺CD4^{low} subjects. In the first approach (FIG. 9a), CD8⁺ T cells were depleted from the PBMC population reaching 100% depletion as confirmed by flow cytometry. The peptide-triggered TRAIL responses seen in the PBMC were no longer detectable in CD8 T cell-depleted PBMC. In contrast, CD4 T cell-depleted PBMC showed moderately increased numbers of HIV-peptide-induced TRAIL spots, reflecting the enrichment of CD8⁺ T cells. In the second approach (FIG. 9b), CD8⁺ T cells were enriched to <97% purity and analyzed for reactivity to the individual peptides against which the PBMC responded. The responses seen in the PBMC reproduced in the CD8⁺ T cell population while showing increased frequencies in the latter reflecting the increased frequencies of CD8⁺ T cells in the study cell population.

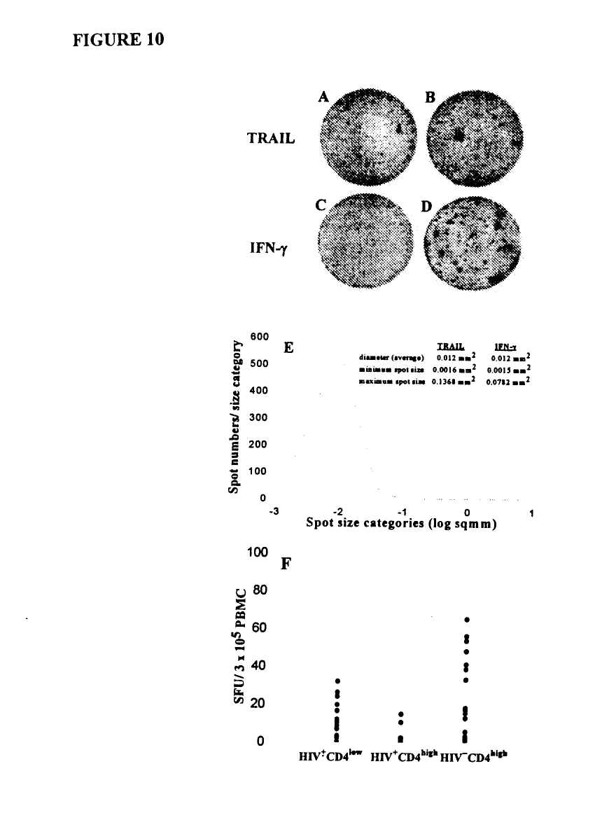

Characteristic TRAIL and IFN-γ spots induced in purified CD8⁺ T cells by HIV peptides are shown (FIGS. 10a and b). The sizes of TRAIL spots were similar for purified CD8⁺ T cells and PBMC. Size and morphology of the TRAIL spots were also similar to IFN-γ spots (FIG. 10, a to d). Spot size histograms are compared in FIG. 10e. Since spot size and morphology in ELISPOT assays reflect the per cell productivity, these data suggest similar per cell TRAIL and IFN-γ productivity. In HIV patients, TRAIL and IFN-γ release occurred over low spontaneous spot formation (FIGS. 10a and c) resulting in a clear signal to noise ratio. Background TRAIL spot formation was regularly elevated in PBMC of healthy donors relative to the HIV-infected subjects (FIG. 101), however in the healthy controls HIV, Hep C and CEF peptides did not induce TRAIL over this spontaneous background (FIGS. 8a and b).

### Example 10

This example describes studies indicating dissociated production of TRAIL and IFN-γ by HIV peptide-specific CD8⁺ T cells.

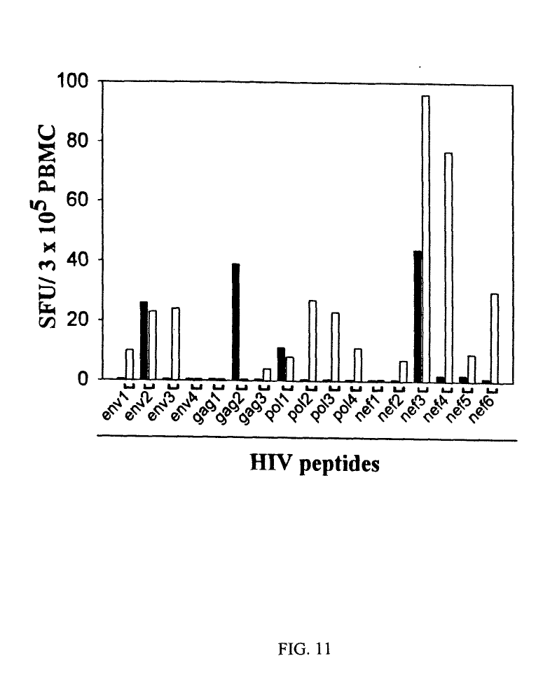

In murine models helped CD8⁺ T cells secrete IFN-γ, but no TRAIL while helpless CD8⁺ T cells release TRAIL and are impaired in IFN-γ production. Twenty-one HIV⁺CD4^{low} subjects were analyzed to determine whether this dichotomy would become detectable at the determinant specificity level. PBMC or CD8⁺ T cells from these donors were challenged with single HIV peptides performing IFN-γ and TRAIL ELISPOT assays in parallel. Peptides that induced TRAIL-producing cells typically did not induce IFN-γ production, and occasionally IFN-γ production was seen in the absence of TRAIL. Mixed responses also occurred, conceivably indicating a heterogeneous CD8⁺ T cell population. A characteristic example is illustrated in FIG. 11 showing TRAIL as well as IFN-γ responses of an individual HIV⁺CD4^{low} subject upon stimulation with HIV peptides derived from diverse viral regions. This TRAIL release profile is consistent with that of helpless CD8⁺ T cells. In seven CD4⁺HIV^{low} subjects with CD4⁺ T cell counts ranging from 0.005% to 0.08%, cumulatively only 5.4% of all HIV peptides analyzed (11/197) triggered an isolated IFN-γ response (Table 1), suggesting that “helplessness/lethargy” affected the majority of the HIV-reactive CD8⁺ T cells in those subjects.

TABLE 1
HIV peptide-induced release of IFN-γ and TRAIL in PBMC
of HIV-infected individuals.

| | HIV-infected subjects, | | | | | | | |
|---|---|---|---|---|---|---|---|---|
| | TRAIL/IFN-γ responses (%) | | | | | | | |
| | A | B | C | D | E | F | G | Mean ± SD |
| IFN-γ | 7 | 10.7 | 4.7 | 7.4 | 0 | 0 | 8.3 | 5.4 ± 3.8 |
| TRAIL | 74.4 | 67.9 | 76.3 | 78.6 | 33.3 | 58.3 | 50 | 62.7 ± 15.3 |
| IFN-γ/ | 18.6 | 21.4 | 19 | 14.3 | 66.7 | 41.7 | 41.7 | 31.9 ± 17.6 |
| TRAIL | | | | | | | | |

PBMC of seven HIV⁺CD4^{low} subjects (A-G) were analyzed in ELISPOT assays for HIV-peptide-induced production of IFN-γ or TRAIL. 78 HIV peptides were individually analyzed in duplicates. Peptides were identified that induced IFN-γ alone (in the absence of TRAIL), TRAIL alone (in the absence of IFN-γ), or the combination of both. For each individual donor the percentage of positive responses to all peptides screened in that particular subject are shown for each category. Mean and SD for all subjects have been calculated and are shown in the last column.

IFN-γ single positive CD8⁺ T cells might reflect determinants of the virus that have undergone mutations—they might have primed CD8⁺ T cells at a time when the host was still CD4^{high}. After the virus mutated, these determinants ceased to induce CD8⁺ T cells recognition. Functionally, such HIV peptides would behave similar to CEF peptides. In contrast, viral peptides that continue to be biosynthesized will cause ongoing immune stimulation. For this reason, the CD8⁺ T cell turnover rate is increased in HIV infection, comparing 1 to 2% in non-HIV-infected individuals to 1 to 10% in HIV-infected patients (Hellerstein et al., Nat Med 5:83 (1999); Sachsenberg et al., J Exp Med 187:1295 (1998)). Most CD8⁺ T cells in HIV⁺CD4^{low} subjects displayed the helpless phenotype suggesting a commitment of CD8⁺ T cells to the helped phenotype which is less stable, as was proposed based on the murine data. Studies of secondary CD8⁺ T cell responses were confined to one or few rounds of re-stimulation (Janssen et al., Nature 434:88 (2005); Bourgeois et al., Eur J Immunol 32:2199 (2002); Bourgeois et al., Science 297:2060 (2002); Sun et al., Science 300:339 (2003)). While also in humans the helped CD8⁺ T cell phenotype may be maintained for limited CD8⁺ T cell re-stimulation, e.g., after a booster immunization or a re-infection that is rapidly cleared in a short time, it seems to collapse in the absence of CD4⁺ T cell help under continuous stimulation caused by the persisting virus. Alternatively, helped CD8⁺ T cells might become exhausted due to viral persistence and thus decline in numbers. In this scenario, the HIV-reactive CD8⁺ T cell pool in HIV⁺CD4^{low} individuals primarily consists of recent thymic emigrants that have already been primed in a CD4⁺ T cell-deficient environment. Whichever mechanism may apply, the data clearly shows that the HIV-specific CD8⁺ T cells (but not the CEF-reactive CD8⁺ T cells) acquire the helpless phenotype in HIV⁺CD4^{low} subjects.

The helpless phenotype is likely as well to affect the ability of the HIV-specific CD8⁺ T cells to control HIV replication. Such cells produce decreased IFN-γ which inhibits viral replication (Creery et al., Clin Exp Immunol 137:156 (2004); Sarol et al., Biochem Biophys Res Commun 291:890 (2002)). If helpless cells behave identically in mice and man, they might have diminished cytolytic activity, in addition to reduced proliferative potential. Increased levels of soluble TRAIL have been measured in the serum of HIV-infected individuals (Herbeuval et al., Blood 105:2458 (2005); Liabakk et al., J Immunol Methods 259:119 (2002)). While monocytes and CD4⁺ T cells have been implicated in producing TRAIL (Herbeuval et al., Blood 105:2458 (2005); Herbeuval et al., Blood 106:3524 (2005)), the data indicates that the HIV-specific CD8⁺ T cells might be a primary source of it.

Since TRAIL has pro-apoptotic effects on virus-infected cells (Ishikawa et al., J Virol 79:7658 (2005)) its release by HIV-specific CD8⁺ T cells might be considered an anti-viral effector function, possibly complimenting perforin and granzyme, Fas-FasL and other TNF superfamily member-mediated cytolytic functions. This tissue culture shows that exposure of HIV-infected cells to TRAIL preferentially kills HIV-infected cells. Injection of TRAIL has been suggested as a therapeutic strategy (Lum et al., J Viral 75:11128 (2001)). In spite of the high numbers of TRAIL-releasing CD8⁺ T cells in advanced HIV infection (i.e. in HIV⁺CD4^{low} subjects) these CD8⁺ T cells do not seem to be able to control the infection. Therefore, apoptosis induction in HIV-infected cells via the TRAIL pathway does not seem to be a successful effector function of CD8⁺ T cells.

To the contrary, soluble TRAIL has been shown to induce apoptosis in uninfected CD4⁺ T cells (Herbeuval et al., Blood 106:3524 (2005)). It seems likely that the release of TRAIL by high numbers of HIV-specific CD8⁺ T cells further contributes to CD4⁺ T cell-depletion in these subjects. In this way, the drop in CD4+ T cell numbers in HIV infection might underlie a threshold-induced vicious circle: once CD4⁺ T cell numbers drop under 200, HIV-specific CD8⁺ T cells acquire the helpless phenotype and start secreting large amounts of TRAIL. This in turn further decreases CD4⁺ T cell counts.

It has been reported that CD4⁺ T cell counts and serum concentrations of TRAIL are inversely correlated (Herbeuval et al., Blood 106:3524 (2005); Sarol et al., Biochem Biophys Res Commun 291:890 (2002)). The neutralization of TRAIL by an inhibitory molecule, such as an antibody, or the temporary inactivation of CD8⁺ T cells could disrupt this vicious circle. In murine models, once CD8⁺ T cells have been primed in the presence of CD4⁺ T cell help, the memory cells become independent of CD4⁺ T cell help for subsequent booster responses (Rocha et al., Curr Opin Immunol 16:259 (2004)). It will have to be established whether re-infections or booster immunizations of such helped CD8⁺ T cells will maintain their helped phenotype in HIV⁺CD4^{low} subjects. The data provided here suggest that this might be the case.

De novo infections of HIV⁺CD4^{low} subjects, e.g., with HCV, are likely to result in a helpless CD8⁺ T cell response contributing to the inability to control HCV infection. For the same reason, vaccination or immunization of HIV⁺CD4^{low} subjects may not be advisable. Such immunizations will not only fail to induce an effective CD8⁺ T cell response, but commit the antigen-specific CD8⁺ T cell pool towards the helpless phenotype even after the patient's CD4⁺ T cells recover due to therapy. Such immunizations should be postponed until CD4⁺ T cell counts rise >200.

TRAIL-producing CD8⁺ T cells were specific for a multitude of peptides in each HIV-infected subject with different peptides being recognized by different donors. Such diverse repertoires have also been observed measuring IFN-γ in the same patients being characteristic of chronic HIV infection (Allen et al., AIDS 15(5):S117 (2001); Cao et al., J Virol 77:6867 (2003); Dalod et al., J Virol 73:7108 (1999); Draenert et al., J Immunol Methods 275:19 (2003); Kleen et al., AIDS 18:383 (2004)). This diversity of peptide recognition results, among other factors, from HLA-polygenism and -polymorphism in the human population. It requires extensive peptide library studies to obtain comprehensive information on the HIV-specific CD8⁺ T cells pool in each individual. IFN-γ ELISPOT assays have become prevalent in immune monitoring of HIV-specific CD8⁺ T cells since they are well suited for high throughput screening of peptide libraries as well as because of their unsurpassed sensitivity for detecting the low frequency HIV peptide-specific CD8⁺ T cells. While intracellular cytokine staining for IFN-γ can be considered an alternative for IFN-γ ELISPOT assays (measuring the antigen-induced de novo synthesis of the cytokine) TRAIL staining does not suit this purpose because of the constitutive membrane expression of this molecule. TRAIL ELISPOT assays might find their place next to IFN-γ ELISPOT assays for immune monitoring of CD8⁺ T cells in HIV infection, the former detecting the helpless CD8⁺ T cells but missing the helped CD8⁺ T cells, the latter detecting the helped CD8⁺ T cells, but missing the helpless CD8⁺ T cells. Enumerating both cell types at single cell resolution permits a precise determination of the prevalence of either cell type in the host.

Thus, the data shows that in HIV⁺CD4^{low} subjects helpless HIV-specific CD8⁺ T cells emerge as possible mediators and sensitive indicators of the immunodeficiency state. The data not only provide mechanistic insights into the immune pathology of HIV infection, but also defining the helpless state of HIV-infected individuals might have clinical relevance. Immunizations or infections in this state are likely to generate defective CD8⁺ T responses against the antigen or infectious agent.

TABLE 2

| PRODUCT | COMPANY |
|---|---|
| Rabbit Anti-TRAIL Polyclonal Antibody, | ABR--Affinity BioReagents |
| Unconjugated | 4620 Technology Drive, Suite 600 |
| | Golden, CO 80403 |
| | USA |
| Anti-Human TRAIL Monoclonal Antibody, | Active Motif |
| Unconjugated, Clone 55B709.3 | 1914 Palomar Oaks Way, Suite 150 |
| | Carlsbad CA 92008 |
| | USA |
| Anti-Human TRAIL (Apo2L) Antibody, | Antigenix America, Inc. |
| Unconjugated | P.O. Box 2666 |
| | Huntington Station, NY 11746 |
| | USA |
| Anti-Human TRAIL (Apo2L) Polyclonal | Antigenix America, Inc. |
| Antibody | P.O. Box 2666 |
| | Huntington Station, NY 11746 |
| | USA |
| Rabbit Anti-Human DR4/TRAIL-R1 NT | Aviva Systems Biology |
| Polyclonal Antibody | 11180 Roselle Street, Suite 300 |
| | San Diego, CA 92121 |
| | USA |
| Anti-TRAIL Monoclonal Antibody, Biotin | BD Biosciences Pharmingen |
| Conjugated, Clone RIK-2 | 2350 Qume Drive |
| | San Jose, CA 95131 |
| | USA |
| Anti-TRAIL Monoclonal Antibody, | BD Biosciences Pharmingen |
| Unconjugated, Clone B35-1 | 2350 Qume Drive |
| | San Jose, CA 95131 |
| | USA |
| Anti-TRAIL Monoclonal Antibody, | BD Biosciences Pharmingen |
| Unconjugated, Clone N2B2 | 2350 Qume Drive |
| | San Jose, CA 95131 |
| | USA |
| Anti-TRAIL Monoclonal Antibody, | BD Biosciences Pharmingen |
| Unconjugated, Clone RIK-1 | 2350 Qume Drive |
| | San Jose, CA 95131 |
| | USA |
| Armenian Hamster Anti-Mouse TRAIL-R2 | BD Biosciences Pharmingen |
| Monoclonal Antibody, Clone MD5-1 | 2350 Qume Drive |
| | San Jose, CA 95131 |
| | USA |
| Human recombinant soluble TRAIL/APO-2L Kit/ | Bender MedSystems Inc. |
| Enhancer Protein | 849 Hinckley Road |
| | Burlingame, CA 94010 |
| | USA |
| rh TRAIL | Bender MedSystems Inc. |
| | 849 Hinckley Road |
| | Burlingame, CA 94010 |
| | USA |
| Human TRAIL/APO2L, Recombinant | BIODESIGN International |
| | 60 Industrial Park Road |
| | Saco, Maine 04072 |
| | USA |
| Mouse Anti-Human DcR1 (TRAIL-R3) (TRAIL- | BioLegend |
| R3, TRID, LIT) Monoclonal Antibody, Clone | 8395 Camino Santa Fe |
| DJR3 | Suite E |
| | San Diego, CA 92121 |
| | USA |
| Mouse Anti-Human DcR2 (TRAIL-R4) (TRAIL- | BioLegend |
| R4, TRUNDD) Monoclonal Antibody, Clone | 8395 Camino Santa Fe |
| DJR4-1 | Suite E |
| | San Diego, CA 92121 |
| | USA |
| Mouse Anti-Human DcR2 (TRAIL-R4) (TRAIL- | BioLegend |
| R4, TRUNDD) Monoclonal Antibody, Clone | 8395 Camino Santa Fe |
| DJR4-2 | Suite E |
| | San Diego, CA 92121 |
| | USA |
| Mouse Anti-Human DR4 (TRAIL-R1) (TRAIL- | BioLegend |
| R1, Apo-2) Monoclonal Antibody, Clone DJR1 | 8395 Camino Santa Fe |
| | Suite E |
| | San Diego, CA 92121 |
| | USA |
| Mouse Anti-Human DR5 (TRAIL-R2) (TRAIL- | BioLegend |
| R2, KILLER, TRICK2, TNFRSF10B, Ly98) | 8395 Camino Santa Fe |
| Monoclonal Antibody, Clone DJR2-4 (7-8) | Suite E |
| | San Diego, CA 92121 |
| | USA |
| Mouse Anti-Human DR5 (TRAIL-R2) (TRAIL- | BioLegend |
| R2, KILLER, TRICK2, TNFRSF10B, Ly98) | 8395 Camino Santa Fe |
| Monoclonal Antibody, Clone DJR2-2 (2-6) | Suite E |
| | San Diego, CA 92121 |
| | USA |
| Anti-Human DR-4/TRAIL-R1 Antibody | Biosource International |
| | 542 Flynn Road |
| | Camarillo California 93012 |
| | USA |
| Mouse Anti-Human TRAIL Monoclonal | Biosource International |
| Antibody, Unconjugated, Clone B-T24 | 542 Flynn Road |
| | Camarillo California 93012 |
| | USA |
| TRAIL | Biosource International |
| | 542 Flynn Road |
| | Camarillo, California 93012 |
| | USA |
| Mouse Anti-TRAIL (APO-2L) Monoclonal | BioVendor Laboratory Medicine, Inc. |
| Antibody, Clone 2E5 | Palackeho tr. 56 |
| | 612 00 Brno |
| | Czech Republic |
| Rabbit Anti-TRAIL Polyclonal Antibody | BioVision |
| | 980 Linda Vista Avenue |
| | Mountain View, California 94043 |
| | USA |
| Human TRAIL/Apo2L | BioVision |
| | 980 Linda Vista Avenue |
| | Mountain View, California 94043 |
| | USA |
| Goat Anti-Human TRAIL Receptor-2, N- | Calbiochem |
| Terminal (68-94) Polyclonal Antibody | EMD Biosciences, Inc. |
| | P.O. Box 12087 |
| | La Jolla, CA 92039-2087 |
| | USA |
| Goat Anti-Human TRAIL Receptor-3, N- | Calbiochem |
| Terminal (73-103) Polyclonal Antibody | EMD Biosciences, Inc. |
| | P.O. Box 12087 |
| | La Jolla, CA 92039-2087 |
| | USA |
| Mouse Anti-Human TRAIL Receptor-2 (DR5) | Calbiochem |
| Monoclonal Antibody, Clone 21D8.F6 | EMD Biosciences, Inc. |
| | P.O. Box 12087 |
| | La Jolla, CA 92039-2087 |
| | USA |
| Rabbit Anti-Human DR4/TRAIL Receptor-1, C- | Calbiochem |
| Terminal (427-445) Polyclonal Antibody | EMD Biosciences, Inc. |
| | P.O. Box 12087 |
| | La Jolla, CA 92039-2087 |
| | USA |
| Rabbit Anti-Human DR4/TRAIL Receptor-1, N- | Calbiochem |
| Terminal (1-20) Polyclonal Antibody | EMD Biosciences, Inc. |
| | P.O. Box 12087 |
| | La Jolla, CA 92039-2087 |
| | USA |
| Rabbit Anti-Human TRAIL Receptor-1 (DR4) | Calbiochem |
| Polyclonal Antibody | EMD Biosciences, Inc. |
| | P.O. Box 12087 |
| | La Jolla, CA 92039-2087 |
| | USA |
| Rabbit Anti-Human TRAIL Receptor-2 (DR5) | Calbiochem |
| Polyclonal Antibody | EMD Biosciences, Inc. |
| | P.O. Box 12087 |
| | La Jolla, CA 92039-2087 |
| | USA |
| Rabbit Anti-Human TRAIL Receptor-4 (249-263) | Calbiochem |
| Polyclonal Antibody | EMD Biosciences, Inc. |
| | P.O. Box 12087 |
| | La Jolla, CA 92039-2087 |
| | USA |
| Rabbit Anti-Human TRAIL Receptor-4 (DcR2) | Calbiochem |
| Polyclonal Antibody | EMD Biosciences, Inc. |
| | P.O. Box 12087 |
| | La Jolla, CA 92039-2087 |
| | USA |
| Rabbit Anti-Human TRAIL, C-Terminal (261-277) | Calbiochem |
| Polyclonal Antibody | EMD Biosciences, Inc. |
| | P.O. Box 12087 |
| | La Jolla, CA 92039-2087 |
| | USA |
| TRAIL, Human, Recombinant, E. coli | Calbiochem |
| | EMD Biosciences, Inc. |
| | P.O. Box 12087 |
| | La Jolla, CA 92039-2087 |
| | USA |
| Anti-Human TRAIL Polyclonal Antibody | Cayman Chemical Company |
| | 1180 East Ellsworth Road |
| | Ann Arbor, Michigan 48108 |
| | USA |
| Anti-Human DcR1/TRAIL-R3 (ED) Polyclonal | Cell Sciences, Inc. |
| Antibody | 480 Neponset Street, Building 12A |
| | Canton, MA 02021 |
| | USA |
| Anti-Human DcR1/TRAIL-R3 (ED2) Polyclonal | Cell Sciences, Inc. |
| Antibody | 480 Neponset Street, Building 12A |
| | Canton, MA 02021 |
| | USA |
| Anti-Human DcR2/TRAIL-R4 (ID) Polyclonal | Cell Sciences, Inc. |
| Antibody | 480 Neponset Street, Building 12A |
| | Canton, MA 02021 |
| | USA |
| Anti-Human DR4/TRAIL-R1 (CT) Polyclonal | Cell Sciences, Inc. |
| Antibody | 480 Neponset Street, Building 12A |
| | Canton, MA 02021 |
| | USA |
| Anti-Human DR4/TRAIL-R1 (NT) Polyclonal | Cell Sciences, Inc. |
| Antibody | 480 Neponset Street, Building 12A |
| | Canton, MA 02021 |
| | USA |
| Anti-Human DR5/TRAIL-R2 (CT Polyclonal | Cell Sciences, Inc. |
| Antibody | 480 Neponset Street, Building 12A |
| | Canton, MA 02021 |
| | USA |
| Anti-Human TRAIL/Apo-2L (CT) Polyclonal | Cell Sciences, Inc. |
| Antibody | 480 Neponset Street, Building 12A |
| | Canton, MA 02021 |
| | USA |
| Anti-Human TRAIL/Apo2L Polyclonal | Cell Sciences, Inc. |
| Antibody | 480 Neponset Street, Building 12A |
| | Canton, MA 02021 |
| | USA |
| Anti-Human TRAIL Monoclonal Antibody, | Cell Sciences, Inc. |
| Clone B-S23 | 480 Neponset Street, Building 12A |
| | Canton, MA 02021 |
| | USA |
| Anti-Human TRAIL Monoclonal Antibody, | Cell Sciences, Inc. |
| Clone B-T24 | 480 Neponset Street, Building 12A |
| | Canton, MA 02021 |
| | USA |
| Anti-Mouse DcR1/TRAIL-R3 (ED) Polyclonal | Cell Sciences, Inc. |
| Antibody | 480 Neponset Street, Building 12A |
| | Canton, MA 02021 |
| | USA |
| Anti-Mouse DcR1/TRAIL-R3 (ED2) Polyclonal | Cell Sciences, Inc. |
| Antibody | 480 Neponset Street, Building 12A |
| | Canton, MA 02021 |
| | USA |
| Anti-Mouse DcR2/TRAIL-R4 (ID) Polyclonal | Cell Sciences, Inc. |
| Antibody | 480 Neponset Street, Building 12A |
| | Canton, MA 02021 |
| | USA |
| Anti-Mouse DR5/TRAIL-R2 (CT Polyclonal | Cell Sciences, Inc. |
| Antibody | 480 Neponset Street, Building 12A |
| | Canton, MA 02021 |
| | USA |
| Anti-Rat DcR1/TRAIL-R3 (ED) Polyclonal | Cell Sciences, Inc. |
| Antibody | 480 Neponset Street, Building 12A |
| | Canton, MA 02021 |
| | USA |
| Anti-Rat DcR1/TRAIL-R3 (ED2) Polyclonal | Cell Sciences, Inc. |
| Antibody, Unconjugated | 480 Neponset Street, Building 12A |
| | Canton, MA 02021 |
| | USA |
| Anti-Rat DcR2/TRAIL-R4 (ID) Polyclonal | Cell Sciences, Inc. |
| Antibody, Unconjugated | 480 Neponset Street, Building 12A |
| | Canton, MA 02021 |
| | USA |
| Human TRAIL/Apo2L | Cell Sciences, Inc. |
| | 480 Neponset Street, Building 12A |
| | Canton, MA 02021 |
| | USA |
| TRAIL/Apo2L, Recombinant, Human | CHEMICON International, Inc. |
| | 28820 Single Oak Drive |
| | Temecula, CA 92590 |
| | USA |
| Anti-DcR1 (Trail-R3, TRID, LIT), Extracellular | CHEMICON International, Inc. |
| Polyclonal Antibody, Unconjugated | 28820 Single Oak Drive |
| | Temecula, CA 92590 |
| | USA |
| Anti-DcR2 (TRAIL-R4, TRUNDD), Intracellular | CHEMICON International, Inc. |
| Domain Polyclonal Antibody, Unconjugated | 28820 Single Oak Drive |
| | Temecula, CA 92590 |
| | USA |
| Anti-DR4 (TRAIL-R1), C-terminus Polyclonal | CHEMICON International, Inc. |
| Antibody, Unconjugated | 28820 Single Oak Drive |
| | Temecula, CA 92590 |
| | USA |
| Anti-TRAIL (Apo 2 Ligand, Apo-2L) Polyclonal | CHEMICON International, Inc. |
| Antibody, Unconjugated | 28820 Single Oak Drive |
| | Temecula, CA 92590 |
| | USA |
| Anti-TRAIL (Apo 2 Ligand, Apo-2L), C-terminus | CHEMICON International, Inc. |
| Polyclonal Antibody, Unconjugated | 28820 Single Oak Drive |
| | Temecula, CA 92590 |
| | USA |
| Anti-TRAIL (Apo 2 Ligand, Apo-2L), N- | CHEMICON International, Inc. |
| Terminus Polyclonal Antibody, Unconjugated | 28820 Single Oak Drive |
| | Temecula, CA 92590 |
| | USA |
| Anti-Human TRAIL Polyclonal Antibody, | Delta Biolabs, LLC |
| Unconjugated | 8870 Muraoka Drive |
| | Gilroy, CA 95020 |
| | USA |
| Chicken Anti-TRAIL (TNF-related apoptosis | GenWay Biotech, Inc. |
| inducing ligand) Polyclonal Antibody | 6777 Nancy Ridge Drive |
| | San Diego, CA 92121 |
| | USA |
| Mouse Anti-DR5 Apo-2, Trail-R2, Trick2, killer | IMGENEX Corp. |
| Monoclonal Antibody, Unconjugated, Clone | 11175 Flintkote Ave., Suite E |
| 45B872.1 | San Diego, CA 92121 |
| | USA |
| Mouse Anti-DR5 Apo-2, Trail-R2, Trick2, killer | IMGENEX Corp. |
| Monoclonal Antibody, Unconjugated, Clone | 11175 Flintkote Ave., Suite E |
| 54B1005 | San Diego, CA 92121 |
| | USA |
| Mouse Anti-Trail/Apo-2 Ligand Monoclonal | IMGENEX Corp. |
| Antibody, Unconjugated, Clone 55B709.3 | 11175 Flintkote Ave., Suite E |
| | San Diego, CA 92121 |
| | USA |
| Rabbit Anti-TRAIL Polyclonal Antibody, | IMGENEX Corp. |
| Unconjugated | 11175 Flintkote Ave., Suite E |
| | San Diego, CA 92121 |
| | USA |
| Anti-DcR1/TRAIL Receptor3 Ab-1 Polyclonal | Lab Vision Corporation |
| Antibody, Unconjugated | 47777 Warm Springs Blvd. |
| | Fremont, CA 94539 |
| | USA |
| Rabbit Anti-Human DcR2/TRAIL-R4/ | Lab Vision Corporation |
| TRUNDD Ab-1 Polyclonal Antibody, | 47777 Warm Springs Blvd. |
| Unconjugated | Fremont, CA 94539 |
| | USA |
| Anti-Human DcR1 (TRAIL-R3, TRID, LIT) | Leinco Technologies, Inc. |
| (Decoy Receptor 1 for TRAIL) (ED, a.a. 149-167) | 359 Consort Drive |
| Antibody | St. Louis, Missouri 63011 |
| | USA |
| Anti-Human DcR1 (TRAIL-R3, TRID, LIT) | Leinco Technologies, Inc. |
| (Decoy Receptor 1 for TRAIL) (ED2, a.a. 111-123) | 359 Consort Drive |
| Antibody | St. Louis, Missouri 63011 |
| | USA |
| Anti-Human DcR2 (TRAIL-R4, TRUNDD) | Leinco Technologies, Inc. |
| (Decoy Receptor 2 for TRAIL) (ID) Antibody | 359 Consort Drive |
| | St. Louis, Missouri 63011 |
| | USA |
| Anti-Human DR4 (TRAIL-R1) (Death Receptor | Leinco Technologies, Inc. |
| for TRAIL) (CT) Antibody | 359 Consort Drive |
| | St. Louis, Missouri 63011 |
| | USA |
| Anti-Human DR4 (TRAIL-R1) (Death Receptor | Leinco Technologies, Inc. |
| for TRAIL) (NT) Antibody | 359 Consort Drive |
| | St. Louis, Missouri 63011 |
| | USA |
| Anti-Human DR5 (Apo2, TRAIL-R2, TRICK2, | Leinco Technologies, Inc. |
| KILLER) (CT) Antibody | 359 Consort Drive |
| | St. Louis, Missouri 63011 |
| | USA |
| Anti-Human TNF-Related Apoptosis Inducing | Leinco Technologies, Inc. |
| Ligand/Apo2 Ligand (TRAIL/Apo2L) | 359 Consort Drive |
| Antibody | St. Louis, Missouri 63011 |
| | USA |
| Anti-Human TRAIL (Apo2L) (CT) Antibody | Leinco Technologies, Inc. |
| | 359 Consort Drive |
| | St. Louis, Missouri 63011 |
| | USA |
| Recombinant Human TNF-Related Apoptosis | Leinco Technologies, Inc. |
| Inducing Ligand/Apo2 Ligand (TRAIL/Apo2L) | 359 Consort Drive |
| | St. Louis, Missouri 63011 |
| | USA |
| Anti-Human Trail-2 Receptor Polyclonal | Maine Biotechnolgy Services, Inc. |
| Antibody | 1037R Forest Avenue |
| | Portland, Maine 04103 |
| | USA |
| Anti-Human Trail-3 Receptor Polyclonal | Maine Biotechnolgy Services, Inc. |
| Antibody | 1037R Forest Avenue |
| | Portland, Maine 04103 |
| | USA |
| Goat Anti-Human TRAIL R2 Polyclonal | Novus Biologicals, Inc. |
| Antibody | PO Box 802 |
| | Littleton, CO 80160 |
| | USA |
| Goat Anti-Human TRAIL R3 Polyclonal | Novus Biologicals, Inc. |
| Antibody | PO Box 802 |
| | Littleton, CO 80160 |
| | USA |
| Mouse Anti-Human TRAIL Monoclonal | Novus Biologicals, Inc. |
| Antibody, Clone 2E5 | PO Box 802 |
| | Littleton, CO 80160 |
| | USA |
| Rabbit Anti-Human TRAIL Polyclonal Antibody | Novus Biologicals, Inc. |
| | PO Box 802 |
| | Littleton, CO 80160 |
| | USA |
| Rabbit Anti-TRAIL Polyclonal Antibody | Novus Biologicals, Inc. |
| | PO Box 802 |
| | Littleton, CO 80160 |
| | USA |
| Mouse Anti-Human Trail/Apo 2L (TNF-Related | PeproTech, Inc. |
| Apoptosis Inducing Ligand) Monoclonal | Princeton Business Park |
| Antibody | P.O. Box 275 |
| | Rocky Hill, NJ 08553 |
| | USA |
| Rabbit Anti-Human TRAIL/Apo2L (TNF- | PeproTech, Inc. |
| Related Apoptosis Inducing Ligand) Polyclonal | Princeton Business Park |
| Antibody | P.O. Box 275 |
| | Rocky Hill, NJ 08553 |
| | USA |
| Human Trail/Apo-II Ligand | PeproTech, Inc. |
| | Princeton Business Park, 5 Crescent Ave. #G-2, |
| | P.O. Box 275 |
| | Rocky Hill, New Jersey 08553 |
| | USA |
| Rabbit Anti-DcR1/TRAIL-R3 (ED) Polyclonal | ProSci, Inc |
| Antibody | 12170 Flint Place |
| | Poway, CA 92064 |
| | USA |
| Rabbit Anti-DcR1/TRAIL-R3 (ED2) Polyclonal | ProSci, Inc |
| Antibody | 12170 Flint Place |
| | Poway, CA 92064 |
| | USA |
| Rabbit Anti-DcR2/TRAIL-R4 (ID) Polyclonal | ProSci, Inc |
| Antibody | 12170 Flint Place |
| | Poway, CA 92064 |
| | USA |
| Rabbit Anti-DR4/TRAIL-R1 (CT) Polyclonal | ProSci, Inc |
| Antibody | 12170 Flint Place |
| | Poway, CA 92064 |
| | USA |
| Rabbit Anti-DR4/TRAIL-R1 (NT) Polyclonal | ProSci, Inc |
| Antibody | 12170 Flint Place |
| | Poway, CA 92064 |
| | USA |
| Rabbit Anti-DR5/TRAIL-R2 Polyclonal | ProSci, Inc |
| Antibody | 12170 Flint Place |
| | Poway, CA 92064 |
| | USA |
| Rabbit Anti-TRAIL/Apo-2L (CT) Polyclonal | ProSci, Inc |
| Antibody | 12170 Flint Place |
| | Poway, CA 92064 |
| | USA |
| Anti-Human TRAIL (CT) Antibody | QED Bioscience Inc. |
| | 10919 Technology Place, Suite C |
| | San Diego, CA 92127 |
| | USA |
| Goat Anti-Human TRAIL/TNFSF10 Polyclonal | R&D Systems, Inc. |
| Antibody | 614 McKinley Place NE |
| | Minneapolis, MN 55413 |
| | USA |
| Goat Anti-Human TRAIL R1/TNFRSF10A | R&D Systems, Inc. |
| Polyclonal Antibody | 614 McKinley Place NE |
| | Minneapolis, MN 55413 |
| | USA |
| Goat Anti-Human TRAIL R2/TNFRSF10B | R&D Systems, Inc. |
| Polyclonal Antibody | 614 McKinley Place NE |
| | Minneapolis, MN 55413 |
| | USA |
| Goat Anti-Human TRAIL R3/TNFRSF10C | R&D Systems, Inc. |
| Polyclonal Antibody | 614 McKinley Place NE |
| | Minneapolis, MN 55413 |
| | USA |
| Goat Anti-Human TRAIL R4/TNFRSF10D | R&D Systems, Inc. |
| Polyclonal Antibody | 614 McKinley Place NE |
| | Minneapolis, MN 55413 |
| | USA |
| Goat Anti-Mouse TRAIL/TNFSF10 Polyclonal | R&D Systems, Inc. |
| Antibody | 614 McKinley Place NE |
| | Minneapolis, MN 55413 |
| | USA |
| Goat Anti-Mouse TRAIL R2/TNFRSF10B | R&D Systems, Inc. |
| Polyclonal Antibody | 614 McKinley Place NE |
| | Minneapolis, MN 55413 |
| | USA |
| Mouse Anti-Human TRAIL/TNFSF10 | R&D Systems, Inc. |
| Monoclonal Antibody | 614 McKinley Place NE |
| | Minneapolis, MN 55413 |
| | USA |
| Mouse Anti-Human TRAIL/TNFSF10 | R&D Systems, Inc. |
| Monoclonal Antibody, Clone 75411 | 614 McKinley Place NE |
| | Minneapolis, MN 55413 |
| | USA |
| Mouse Anti-Human TRAIL R1/TNFRSF10A | R&D Systems, Inc. |
| Monoclonal Antibody, Clone 69036 | 614 McKinley Place NE |
| | Minneapolis, MN 55413 |
| | USA |
| Mouse Anti-Human TRAIL R2/TNFRSF10B | R&D Systems, Inc. |
| Monoclonal Antibody, Clone 152415 | 614 McKinley Place NE |
| | Minneapolis, MN 55413 |
| | USA |
| Mouse Anti-Human TRAIL R2/TNFRSF10B | R&D Systems, Inc. |
| Monoclonal Antibody, Clone 71903 | 614 McKinley Place NE |
| | Minneapolis, MN 55413 |
| | USA |
| Mouse Anti-Human TRAIL R2/TNFRSF10B | R&D Systems, Inc. |
| Monoclonal Antibody, Clone 71908 | 614 McKinley Place NE |
| | Minneapolis, MN 55413 |
| | USA |
| Mouse Anti-Human TRAIL R3/TNFRSF10C | R&D Systems, Inc. |
| Monoclonal Antibody, Clone 90906 | 614 McKinley Place NE |
| | Minneapolis, MN 55413 |
| | USA |
| Mouse Anti-Human TRAIL R3/TNFRSF10C | R&D Systems, Inc. |
| Monoclonal Antibody, Clone 90903 | 614 McKinley Place NE |
| | Minneapolis, MN 55413 |
| | USA |
| Mouse Anti-Human TRAIL R3/TNFRSF10C | R&D Systems, Inc. |
| Monoclonal Antibody, Clone 90905 | 614 McKinley Place NE |
| | Minneapolis, MN 55413 |
| | USA |
| Mouse Anti-Human TRAIL R4/TNFRSF10D | R&D Systems, Inc. |
| Monoclonal Antibody, Clone 104918 | 614 McKinley Place NE |
| | Minneapolis, MN 55413 |
| | USA |
| Rabbit Anti-TRAIL R2/TNFRSF10B Polyclonal | R&D Systems, Inc. |
| Antibody | 614 McKinley Place NE |
| | Minneapolis, MN 55413 |
| | USA |
| Rat Anti-Mouse TRAIL/TNFSF10 Monoclonal | R&D Systems, Inc. |
| Antibody, Clone 170533 | 614 McKinley Place NE |
| | Minneapolis, MN 55413 |
| | USA |
| Rat Anti-Mouse TRAIL R2/TNFRSF10B | R&D Systems, Inc. |
| Monoclonal Antibody, Clone 118929 | 614 McKinley Place NE |
| | Minneapolis, MN 55413 |
| | USA |
| TRAIL | R&D Systems, Inc. |
| | 614 McKinley Place NE |
| | Minneapolis, MN 55413 |
| | USA |
| Mouse Anti-Human TRAIL (aa95-281) | Serotec Inc |
| Monoclonal Antibody, Clone 2E5 | 3200 Atlantic Avenue, Suite 105 |
| | Raleigh, NC 27604 |
| | USA |
| Rabbit Anti-TRAIL (CT) Polyclonal Antibody | Serotec Inc |
| | 3200 Atlantic Avenue, Suite 105 |
| | Raleigh, NC 27604 |
| | USA |
| TRAIL/APO2L [HUMAN] (RECOMBINANT) | Serotec Inc. |
| | 3200 Atlantic Avenue, Suite 105 |
| | Raleigh, NC 27604 |
| Mouse Anti-TRAIL Monoclonal Antibody,, | Sigma-Aldrich |
| Clone 75411.11 | 3050 Spruce St. |
| | St. Louis, MO 63103 |
| | USA |
| Rabbit Anti-TRAIL Antibody | Sigma-Aldrich |
| | 3050 Spruce St. |
| | St. Louis, MO 63103 |
| | USA |
| Anti-TRAIL (TNF Related-Apoptosis Ligand) | Stressgen Bioreagents |
| Polyclonal Antibody | 800 Technology Drive |
| | Ann Arbor, Michigan 48108 |
| | USA |
| Anti-Human DcR1 (Decoy Receptors for TRAIL, | United States Biological |
| ED2, TRAIL-R3, TRID, LIT) Polyclonal | P.O. Box 261 |
| Antibody | Swampscott, MA 01907 |
| | USA |
| Anti-Human DcR1 ED (TRAIL-R3, TRID, LIT, | United States Biological |
| Decoy Receptor1) Polyclonal Antibody | P.O. Box 261 |
| | Swampscott, MA 01907 |
| | USA |
| Anti-Human DcR2 (CT) (TRAIL-R4, TRUNDD, | United States Biological |
| Decoy Receptor2) Polyclonal Antibody | P.O. Box 261 |
| | Swampscott, MA 01907 |
| | USA |
| Anti-Human DcR2 (ID) (TRAIL-R4, TRUNDD, | United States Biological |
| Decoy Receptor2) Polyclonal Antibody | P.O. Box 261 |
| | Swampscott, MA 01907 |
| | USA |
| Anti-Human DR4 NT (TRAIL R1, TRAIL | United States Biological |
| Receptor1, Death Receptor4) Polyclonal | P.O. Box 261 |
| Antibody | Swampscott, MA 01907 |
| | USA |
| Anti-Human DR4, CT (TRAIL R1, TRAIL | United States Biological |
| Receptor1, Death Receptor4) Polyclonal | P.O. Box 261 |
| Antibody | Swampscott, MA 01907 |
| | USA |
| Anti-Human DR4, NT (TRAIL R1, TRAIL | United States Biological |
| Receptor1, Death Receptor4) Polyclonal | P.O. Box 261 |
| Antibody | Swampscott, MA 01907 |
| | USA |
| Anti-Human DR5 (Apo2, Trail-R2, TRAIL | United States Biological |
| Receptor2, Trick2, Killer, Death Receptor 5) | P.O. Box 261 |
| Polyclonal Antibody | Swampscott, MA 01907 |
| | USA |
| Anti-Human TRAIL (TNF-Related Apoptosis | United States Biological |
| Inducing Ligand, Apo2L, TL2, TNFSF10) | P.O. Box 261 |
| Monoclonal Antibody, clone 2B2.108 | Swampscott, MA 01907 |
| | USA |
| Anti-Human TRAIL (TNF-Related Apoptosis | United States Biological |
| Inducing Ligand, Apo2L, TL2, TNFSF10) | P.O. Box 261 |
| Polyclonal Antibody | Swampscott, MA 01907 |
| | USA |
| Mouse Anti-TRAIL Monoclonal Antibody | Upstate |
| | 706 Forest Street, Suite 1 |
| | Charlottesville, VA 22903 |
| | USA |
| Rabbit Anti-DR4/TRAIL-R1 NT Polyclonal | Upstate |
| Antibody | 706 Forest Street, Suite 1 |
| | Charlottesville, VA 22903 |
| | USA |

| CODE | NAME | SOURCE | CLONALITY | APPLICATIONS | INFO. |
|---|---|---|---|---|---|
| ab2219 | TRAIL antibody | Mouse | Monoclonal | FuncS | Apo 2 ligand, Apo 2L, |
| | [2E5] | | | | APO2L, TL2, TNF |
| | | | | | Related Apoptosis |
| | | | | | Inducing Ligand, |
| | | | | | TNFSF10 . . . Cross- |
| | | | | | reacts with Human. Not |
| | | | | | yet tested in other |
| | | | | | species. |
| ab3921 | TRAIL antibody | Rabbit | Polyclonal | WB | Apo 2 ligand, Apo 2L, |
| | | | | | APO2L, TL2, TNF |
| | | | | | Related Apoptosis |
| | | | | | Inducing Ligand, |
| | | | | | TNFSF10 . . . Cross- |
| | | | | | reacts with Human. |
| | | | | | Expected to cross-react |
| | | | | | with Chicken (84% |
| | | | | | identity), Mouse (92% |
| | | | | | identity) and Rat (84% |
| | | | | | identity) due to |
| | | | | | sequence homology. |
| | | | | | Not yet tested in other |
| | | | | | species. |
| ab12124 | TRAIL antibody | Mouse | Monoclonal | WB | Apo 2 ligand, Apo 2L, |
| | [55B709.3] | | | | APO2L, TL2, TNF |
| | | | | | Related Apoptosis |
| | | | | | Inducing Ligand, |
| | | | | | TNFSF10 . . . Cross- |
| | | | | | reacts with Human. Not |
| | | | | | yet tested in other |
| | | | | | species. |
| ab2056 | TRAIL antibody | Rabbit | Polyclonal | WB | Apo 2 ligand, Apo 2L, |
| | | | | | APO2L, TL2, TNF |
| | | | | | Related Apoptosis |
| | | | | | Inducing Ligand, |
| | | | | | TNFSF10 . . . Cross- |
| | | | | | reacts with Human. Not |
| | | | | | yet tested in other |
| | | | | | species. |
| ab2435 | TRAIL antibody | Rabbit | Polyclonal | IHC-Fr, IP, WB | Apo 2 ligand, Apo 2L, |
| | | | | | APO2L, TL2, TNF |
| | | | | | Related Apoptosis |
| | | | | | Inducing Ligand, |
| | | | | | TNFSF10. Cross-reacts |
| | | | | | with Human, Rat and |
| | | | | | Mouse. Not yet tested in |
| | | | | | other species. |
| ab9959 | TRAIL antibody | Rabbit | Polyclonal | ELISA, Neut, WB | Apo 2 ligand, Apo 2L, |
| | | | | | APO2L, TL2, TNF |
| | | | | | Related Apoptosis |
| | | | | | Inducing Ligand, |
| | | | | | TNFSF10 Cross-reacts |
| | | | | | with Human. Not yet |
| | | | | | tested in other species. |
| ab12789 | TRAIL antibody | Rabbit | Polyclonal | ELISA, WB | Apo 2 ligand, Apo 2L, |
| | | | | | APO2L, TL2, TNF |
| | | | | | Related Apoptosis |
| | | | | | Inducing Ligand, |
| | | | | | TNFSF10 . . . Cross-reacts |
| | | | | | with Human. Not yet |
| | | | | | tested in other species. |
| ab9959 | TRAIL antibody | Rabbit | Polyclonal | ELISA, Neut, WB | Apo 2 ligand, Apo 2L, |
| | | | | | APO2L, TL2, TNF |
| | | | | | Related Apoptosis |
| | | | | | Inducing Ligand, |
| | | | | | TNFSF10 Cross-reacts |
| | | | | | with Human. Not yet |
| | | | | | tested in other species. |
| ab10516 | TRAIL antibody | Mouse | Monoclonal | ELISA, Inhib, | Apo 2 ligand, Apo 2L, |
| | [75411.11] | | | Neut, | APO2L, TL2, TNF |
| | | | | WB | Related Apoptosis |
| | | | | | Inducing Ligand, |
| | | | | | TNFSF10 . . . Cross-reacts |
| | | | | | with Human. Not yet |
| | | | | | tested in other species. |
| ab2036 | TNFRSF10C | Rabbit | Polyclonal | WB | Antagonist decoy |
| | antibody | | | | receptor for TRAIL Apo |
| | | | | | 2L, DCR1, Decoy |
| | | | | | receptor 1, Decoy |
| | | | | | TRAIL . . . Cross-reacts |
| | | | | | with Human. Not yet |
| | | | | | tested in other species. |
| ab5975 | DcR1 antibody | Rabbit | Polyclonal | WB | DcR 1, Decoy Receptor |
| | | | | | 1, TNF Related |
| | | | | | Apoptosis Inducing |
| | | | | | Ligand Receptor 3, |
| | | | | | TNFRS . . . Cross-reacts |
| | | | | | with Human, Mouse and |
| | | | | | Rat. Not yet tested in |
| | | | | | other species. |
| ab2087 | DcR1 antibody | Rabbit | Polyclonal | WB | DcR 1, Decoy Receptor |
| | | | | | 1, TNF Related |
| | | | | | apoptosis. |
| ab5808 | DcR1 antibody | Rabbit | Polyclonal | WB | DcR 1, Decoy Receptor |
| | | | | | 1, TNF Related |
| | | | | | Apoptosis Inducing |
| | | | | | Ligand Receptor 3, |
| | | | | | TNFRS Cross-reacts |
| | | | | | with Human and Rat. |
| | | | | | Not yet tested in other |
| | | | | | species. |
| ab5693 | DcR1 antibody | Rabbit | Polyclonal | WB | DcR 1, Decoy Receptor |
| | | | | | 1, TNF Related |
| | | | | | Apoptosis Inducing |
| | | | | | Ligand Receptor 3, |
| | | | | | TNFRS . . . Cross-reacts |
| | | | | | with Human, Mouse and |
| | | | | | Rat. Not yet tested in |
| | | | | | other species. |
| ab13863 | DcTRAILR1 | Rabbit | Polyclonal | WB | Dctrailr1, Decoy TRAIL |
| | antibody | | | | receptor 1, mSOB, TNF |
| | | | | | receptor family member |
| | | | | | SOB, TNF rec . . . Cross- |
| | | | | | reacts with Mouse. Not |
| | | | | | yet tested in other |
| | | | | | species. |
| ab16092 | DcTRAILR2 | Rat | Monoclonal | FACS | Decoy TRAIL receptor |
| | antibody [Lucy-1] | | | | 2, TNF receptor family |
| | | | | | member SOBa, TNF |
| | | | | | receptor homolog |
| | | | | | 2,.Cross-reacts with |
| | | | | | Mouse. Not yet tested in |
| | | | | | other species. |
| ab2019 | DcR2 antibody | Rabbit | Polyclonal | WB | Decoy Receptor 2, ID |
| | | | | | Cross-reacts with |
| | | | | | Human, Rat and Mouse. |
| | | | | | Not yet tested in other |
| | | | | | species. |
| ab1675 | TRAIL Receptor | Goat | Polyclonal | ELISA, FACS, | Cross-reacts with |
| | 2 antibody | | | ICC, WB | Human. Not yet tested |
| | | | | | in other species. |
| ab1674 | TRAIL Receptor | Goat | Polyclonal | ELISA, FACS, | Cross-reacts with |
| | 3 antibody | | | ICC, WB | Human. Not yet tested |
| | | | | | in other species. |
| ab8405 | DcR3 antibody | Rabbit | Polyclonal | WB | Decoy Receptor 3, TR6 |
| | | | | | Cross-reacts with |
| | | | | | Human, Rat and Mouse. |
| | | | | | Not yet tested in other |
| | | | | | species. |
| ab2061 | DR4 antibody | Rabbit | Polyclonal | WB | Apo2, Cytotoxic ligand |
| | | | | | TRAIL receptor, |
| | | | | | Cytotoxic TRAIL |
| | | | | | receptor, Death receptor . . . |
| | | | | | Cross-reacts with |
| | | | | | Human. Not yet tested |
| | | | | | in other species. |
| ab13890 | DR4 antibody | Mouse | Monoclonal | WB | Apo2, Cytotoxic ligand |
| | [32A242] | | | | TRAIL receptor, |
| | | | | | Cytotoxic TRAIL |
| | | | | | receptor, Death receptor. |
| | | | | | Cross-reacts with |
| | | | | | Human. Not yet tested |
| | | | | | in other species. |
| ab8415 | DR4 antibody | Rabbit | Polyclonal | WB | Apo2, Cytotoxic ligand |
| | | | | | TRAIL receptor, |
| | | | | | Cytotoxic TRAIL |
| | | | | | receptor, Death receptor |
| | | | | | Cross-reacts with |
| | | | | | Human. Not yet tested |
| | | | | | in other species. |
| ab8414 | DR4 antibody | Rabbit | Polyclonal | IHC-P, WB | Apo2, Cytotoxic ligand |
| | | | | | TRAIL receptor, |
| | | | | | Cytotoxic TRAIL |
| | | | | | receptor, Death receptor . . . |
| | | | | | Cross-reacts with |
| | | | | | Human, Rat and Mouse. |
| | | | | | Not yet tested in other |
| | | | | | species. |
| | | | | | Inducing Ligand |
| | | | | | Receptor 3, TNFRS . . . |
| | | | | | Cross-reacts with |
| | | | | | Human, Mouse and Rat. |
| | | | | | Not yet tested in other |
| | | | | | species. |
| ab11931 | DR4 antibody | Mouse | Monoclonal | WB | Apo2, Cytotoxic ligand |
| | [32A1380] | | | | TRAIL receptor, |
| | | | | | Cytotoxic TRAIL |
| | | | | | receptor, Death receptor . . . |
| | | | | | Cross-reacts with |
| | | | | | Human. Not yet tested |
| | | | | | in other species. |
| ab14738 | DR4 antibody | Mouse | Monoclonal | FACS, FuncS, IP | Apo2, Cytotoxic ligand |
| | [DR-4-02] - | | | | TRAIL receptor, |
| | Azide free | | | | Cytotoxic TRAIL |
| | | | | | receptor, Death receptor. |
| | | | | | Cross-reacts with |
| | | | | | Human. Not yet tested |
| | | | | | in other species. |
| ab16329 | DR5 antibody | Rabbit | Polyclonal | WB | Apoptosis inducing |
| | | | | | protein, Apoptosis |
| | | | | | inducing receptor, |
| | | | | | Cytotoxic TRAIL |
| | | | | | recepto . . . Cross-reacts |
| | | | | | with Human and Rat. |
| | | | | | Not yet tested in other |
| | | | | | species. |
| ab4106 | DR5 antibody | Rabbit | Polyclonal | WB | Apoptosis inducing |
| | | | | | protein, Apoptosis |
| | | | | | inducing receptor, |
| | | | | | Cytotoxic TRAIL |
| | | | | | receptor. Cross-reacts |
| | | | | | with Human. Not yet |
| | | | | | tested in other species. |
| ab11932 | DR5 antibody | Mouse | Monoclonal | WB | Apoptosis inducing |
| | [54B1005] | | | | protein, Apoptosis |
| | | | | | inducing receptor, |
| | | | | | Cytotoxic TRAIL |
| | | | | | recepto . . . Cross-reacts |
| | | | | | with Human. Not yet |
| | | | | | tested in other species. |
| ab8416 | DR5 antibody | Rabbit | Polyclonal | IHC-P, WB | Apoptosis inducing |
| | | | | | protein, Apoptosis |
| | | | | | inducing receptor, |
| | | | | | Cytotoxic TRAIL |
| | | | | | recepto . . . Cross-reacts |
| | | | | | with Human and Mouse. |
| | | | | | Not yet tested in other |
| | | | | | species. |
| ab8417 | DR6 antibody | Rabbit | Polyclonal | WB | BM018, Death receptor |
| | | | | | 6, TNFR related; Cross- |
| | | | | | reacts with Human. Not |
| | | | | | yet tested in other |
| | | | | | species. |

1-34. (canceled)

35. A method of promoting or inducing apoptosis or death of T cells, comprising contacting T cells with an amount of an activator of TRAIL receptor (DR4 or DR5) expression or activity sufficient to promote or induce apoptosis or death of T cells.

36. A method of treating a physiological condition, disorder, illness, disease or symptom of a subject that is ameliorated by promoting or inducing T cell apoptosis or death, comprising administering an amount of an activator of TRAIL receptor (DR4 or DR5) expression or activity effective to promote or induce T cell apoptosis or death, thereby ameliorating the physiological condition, disorder, illness, disease or symptom.

37. The method of claim 36, wherein activated CD8+ T cells contribute to, stimulate, enhance or mediate the physiological disorder or disease.

38. The method of claim 36, wherein the physiological condition, disorder, illness, disease or symptom comprises an autoimmune disorder or disease.

39. The method of claim 36, wherein the physiological disorder or disease comprises multiple sclerosis, autoimmune diabetes, autoimmune hepatitis, primary biliary cirrhosis, myelodysplastic syndrome, aplastic anemia or polymyostitis.

40. The method of claim 36, wherein the physiological disorder or disease comprises transplant rejection or graft-versus-host disease.

41-127. (canceled)

128. The method of claim 35 or 36, wherein the activator of TRAIL receptor (DR4 or DR5) is selected from Table 2.

129. The method of claim 35 or 36, wherein the activator of TRAIL receptor comprises an agonist antibody that specifically binds TRAIL (Apo-2L) or TRAIL receptor (DR4 or DR5).

130. The method of claim 129, wherein the antibody comprises a monoclonal antibody.

131. The method of claim 129, wherein the antibody comprises an IgG, IgA, IgM, IgE or IgD.

132. The method of claim 131, wherein the IgG is selected from IgG₁, IgG₂, IgG₃, and IgG₄.

133. The method of claim 129, wherein the antibody is human or humanized.

134. The method of claim 129, wherein the antibody comprises an antibody fragment that specifically binds TRAIL (Apo-2L) or TRAIL receptor (DR4 or DR5).

135. The method of claim 134, wherein the fragment that specifically binds TRAIL (Apo-2L) or TRAIL receptor (DR4 or DR5) comprises a single-chain Fv, Fab′, (Fab′)₂, Fd, disulfide-linked Fv, light chain variable (VL) or heavy chain variable (VH) sequence.

136. The method of claim 35 or 36, wherein the activator of TRAIL receptor comprises a TRAIL (Apo-2L) chimera.

137. The method of claim 136, wherein the TRAIL (Apo-2L) chimera comprises a polypeptide sequence.

138. The method of claim 137, wherein the polypeptide sequence further comprises an immunoglobulin sequence.


SEQUENCE LISTING (19 sequences)

SEQ ID NO: 1 - protein, 281 aa, Homo sapiens [sequence omitted]

SEQ ID NO: 2 - protein, 291 aa, Mus musculus
MPSSGALKDLSFSQHFRMMVICIVLLQVLLQAVSVAVTYMYFTNEMKQLQDNYSKIGLACFSKTDEDFWDSTDGEILNRPCLQVKRQLYQLIEEVTLRTFQDTISTVPEKQLSTPPLPRGGRPQKVAAHITGITRRSNSALIPISKDGKTLGQKIESWESSRKGHSFLNHVLFRNGELVIEQEGLYYIYSQTYFRFQEAEDASKMVSKDKVRTKQLVQYIYKYTSYPDPIVLMKSARNSCWSRDAEYGLYSIYQGGLFELKKNDRIFVSVTNEHLMDLDQEASFFGAFLIN

SEQ ID NO: 3 - DNA, 1769 bp, Homo sapiens
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 4 - DNA, 4944 bp, Mus musculus
aactgtgaccttctcaggcactgctgctgggctgcaagtctgcattgggaagtcagacctggacagcagtatgccttcctcaggggccctgaaggacctcagcttcagtcagcacttcaggatgatggtgatttgcatagtgctcctgcaggtgctcctgcaggctgtgtctgtggctgtgacttacatgtacttcaccaacgagatgaagcagctgcaggacaattactccaaaattggactagcttgcttctcaaagacggatgaggatttctgggactccactgatggagagatcttgaacagaccctgcttgcaggttaagaggcaactgtatcagctcattgaagaggtgactttgagaacctttcaggacaccatttctacagttccagaaaagcagctaagtactcctcccttgcccagaggtggaagacctcagaaagtggcagctcacattactgggatcactcggagaagcaactcagctttaattccaatctccaaggatggaaagaccttaggccagaagattgaatcctgggagtcctctcggaaagggcattcatttctcaaccacgtgctctttaggaatggagagctggtcatcgagcaggagggcctgtattacatctattcccaaacatacttccgatttcaggaagctgaagacgcttccaagatggtctcaaaggacaaggtgagaaccaaacagctggtgcagtacatctacaagtacaccagctatccggatcccatagtgctcatgaagagcgccagaaacagctgttggtccagagatgccgagtacggactgtactccatctatcagggaggattgttcgagctaaaaaaaaatgacaggatttttgtttctgtgacaaatgaacatttgatggacctggatcaagaagccagcttctttggagcctttttaattaactaaatgaccagtaaagatcaaacacagccctaaagtacccagtaatcttctaggttgaaggcatgcctggaaagcgactgaactggttaggatatggcctggctgtagaaacctcaggacagatgtgacagaaaggcagctggaactcagcagcgacaggccaacagtccagccacagacactttcggtgtttcatcgagagacttgctttctttccgcaaaatgagatcactgtagcctttcaatgatctacctggtatcagtttgcagagatctagaagacgtccagtttctaaatatttatgcaacaattgacaattttcacctttgttatctggtccaggggtgtaaagccaagtgctcacaggctgtgtgcagaccaggatagctatgaatgcaggtcagcataaaaatcacagaatatctcacctaccaaatcagagtgggtgtgcccctgtgtgtatatgcgtgtctgtgtgtgtgtgcatgtatgtgtgtgtgtgtgtgactgttctttatggtaactggttatgtttttctcaagtgaaaaacataactctatacatgataacataatatcccatcatcagtggaaccttgcccaaagaatgtatgaaatctccaggcaatgaatgagggcagcccaagaaagaggcccgcagagccataccacagggctgccccaccctgctggagctcagatcctgccactgctgcaggccctgggtaccaggtgtagagttggaggaggtcttgcctgtgggtctaggtctttggtgcctacctccttgatatggccccagtcctcctttgcttgtttgctagttttatcatgtttcccaggccggcctcaagtccaatatgtagtcaagagtgatctctaactgtgcaacctcctgcctccaagatctgctgagattataggcatgtgcccccctgtctgatttgtgtagagccaggcttcttgtacatgtgacaaccatgccaccctcagctctgtcccagctccatttcttcctttctgaatgcaagcatttactttgtgtccctatattctagaatgtgcaacagtgaagaatttgctctgactttcaggataaagtttgaactaggttcaccatgcttgctttgtccagattgcgactgtcacccagtcctctggctcttccatctgtctgtccactccacctaccaagatgttgaacacttgttctttttaagatgttggtgcctggagtttcattagagtaacacaaaactaactaaaaccaaacaactccaaaggagcccatatgtgttttaatgaaacattttttaagcctattgggggcctgaagagattgctcagaggaaaacagcacttccagaggacccaggttcaattctcatcgctgatgtgatagttaacagctgtaacttcagttccaaggggtctgactttctgccctttgcttgcaatgcatgtatgtgatacacagacatacattctgacaaaatatatccatacacaaaagtatttttttaaaagcttatttgaatgtaagagtatggctagctgtcacttctgataccccttcttatttttttatgactcaagcccttataaactagcaaatagaagtcacagctaccacttgaatataagcacttgaatacctcctctcactagaatacaacatagcttaatagtaaaaatcttgccttagtaaagtacttgcatgtcatgtctacatgaaccaaatgaatgtattaattaataatagacataatgatcacatcggaaaggctgtgagaaataatggagaacatttgaaagctcaagatggaagggaaaggcacttgtcaaaaatcttgacaacctgaatttgacctttggcagggctgaaaactaaacccagggtcttactcccagtaggcatgaacccccacactgagcggcaccacagccctaaggttttaaatggcagaaacaactgagcgcttttctaattcctggtccccaagtcatcactgatggcaaaattcacagcacttcatcttgtctgccaagctggcaagcgaaatggtgtctgatttcttttttgtttttttaaagatttattcattattatatctaagtacactgtagctgtcttcagatgcaccagaaaagggcgtcagatctcattacggatggttgtgagccaccatgtggttgctgggatttgaactcagaaacttcagaagagcagtcggtgctcttaaccactgagccacctctccagccccagtgtctgatttctaatgtttgtgttgaacgtatacattctttgtctgctgttaattctatagtaatctcctgtgaaagattttataacgcccaggcctggattcaaccacaatccaacaggatcggaatcctggggtggtgctcagagatctttttgtgcgtgcattttagttttttaaagtaattagcaggcagcctgcaggacacatctttgccaaaatgtcaaatgctgtgacttataaattaaatatttaagaatgcagcatacccagagaaatcattagatgttcaaagatttaattcgtgtgcaacattaaatgccatgacttgtttaaacaagcacacctaaatattctttcttctgctagaatctgaagcctgtgatgcaggaagcaagaatggagagaatattctagaataagaactttctgggcgattgtgagccctttaataaaaggcatgaatacttatctgtttgaaagagaaatcaatttggggtgggggcgggggttgtcctttctttgctgagttctagcccaacaagccatctgacttgaagactaagacgagcctgccacctgggcaagcacagggacaatgatcccagggcagaggccagctctgctgttttgagaagtgtctaggcaaaagctttctttctttttttttttttttcagaccaaaaggggcctctttaatgtcacacaccattagtgccaccaggtggcgaggttcccttaatttcttcagtatcaagtcttttaaaagtgtatgagtataaacatcttcaaattctcgggtttaaaaacaaaatcaaaaatgaaaaaaggtcgcttacctttagaccataggcaactggagctaaagacaagagataagctggtgtaccccgatggagcagcaggctttaactgaatctgtgaggaacagggtggggcttcacatggtaaaactgaatctgaggtgtgttcttttgcctgtttgggttgttgctgttgttactgctttgttttgtttttttgtttttaagtgaaacagaatttaagtcttttggaagacttttgtaaattctcttggtcccaagggagggctctgtgacgtctggagtctcaacagtttggtctttgccctgtctcctgttattatagactgtggcctttgccactccatcactgcctactgtttttcttggagacagggtctctcccattacgggcagccagcagtgccctgagagtctcttgacatgtggccatgaaggctttttacaaggatgctggggatttgaacccgggttctcatgcttgagccataactgctcttaccctagaactgtctccccagtccaatcactgctactatttttttttcttggcaaatcaattcagttctttagttttctttcaagtattgttttacctatttatcatatttatcactttcattatcaatgttagaaattttgtttagccagcctttttacttattcttgaaataaagggttaaatagatattttttcagttgtgagacatgaaaatcttagaaagaatttttcttttctttttcctttcattttaatagcagtgaatctagaaagagaattatttttcatggtgattatttcaaatgtattgataccattttgtgggctgggaaatgttaaactttgtaaactctgaaacgcacaaagtgttgctttgaatttcaccttaataaaaataacatcaagta

SEQ ID NO: 5 - DNA, 1407 bp, Homo sapiens
atggcgccaccaccagctagagtacatctaggtgcgttcctggcagtgactccgaatcccgggagcgcagcgagtgggacagaggcagccgcggccacacccagcaaagtgtggggctcttccgcggggaggattgaaccacgaggcgggggccgaggagcgctccctacctccatgggacagcacggacccagtgcccgggcccgggcagggcgcgccccaggacccaggccggcgcgggaagccagccctcggctccgggtccacaagaccttcaagtttgtcgtcgtcggggtcctgctgcaggtcgtacctagctcagctgcaaccatcaaacttcatgatcaatcaattggcacacagcaatgggaacatagccctttgggagagttgtgtccaccaggatctcatagatcagaacgtcctggagcctgtaaccggtgcacagagggtgtgggttacaccaatgcttccaacaatttgtttgcttgcctcccatgtacagcttgtaaatcagatgaagaagagagaagtccctgcaccacgaccaggaacacagcatgtcagtgcaaaccaggaactttccggaatgacaattctgctgagatgtgccggaagtgcagcacagggtgccccagagggatggtcaaggtcaaggattgtacgccctggagtgacatcgagtgtgtccacaaagaatcaggcaatggacataatatatgggtgattttggttgtgactttggttgttccgttgctgttggtggctgtgctgattgtctgttgttgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggagtga

SEQ ID NO: 6 - DNA, 1236 bp, Homo sapiens
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaa

SEQ ID NO: 7 - DNA, 3142 bp, Mus musculus
gtcgccgcgacaagaatccagaacttttctgggagtgaggaaatccagagaacttttttaggagtgaggggacagccatccttcgtggcttttgggagctgaagccgcagggtttcggatgagctgacaccatggagcctccaggacccagcacgcccacagcctctgccgctgcccgggcagatcactacaccccaggcctccggccactcccgaagcgcagacttctatatagctttgcgttgctgcttgctgtgctacaggctgtctttgttccagtaacagctaacccagcccataatcgtccagctggcctacagcggccggaggagagcccatcaagaggaccctgtctagcaggccagtacctgtcagaagggaactgcaagccttgcagagagggtattgactacaccagccattccaaccattctctggattcatgtattctctgcacagtctgtaaggaagataaagtcgtagaaacccgatgcaacataaccacaaatacggtgtgtcgatgcaaaccaggcacctttgaagataaagactcccctgagatctgccagtcatgctctaactgcactgacggggaagaggaactgacttcctgtacccccagagaaaaccggaagtgtgtctccaaaacggcttgggcatcttggcataagctaggcctctggataggactcctggttccagtagtgctgctgattggagctctgcttgtctggaagactggagcatggaggcaatggttgctctgtataaaaagaggctgtgaacgggatcccgaaagtgcgaactctgtgcattcgtctctcttggaccgacagacatctagcacgacaaatgactctaaccacaacacggaacctggcaagactcagaaaacaggaaagaagttgctggttccggtaaacggaaacgactcagctgacgacctgaagtttatcttcgagtattgttcggacatagtgccctttgactcctggaaccgtctcatgcggcagttgggcctcacagacaatcaaatccaaatggtcaaagccgaaacactggtcacacgtgaggccctgtaccaaatgctgctcaagtggcgccaccagactgggcgaagtgcctccatcaaccatctgctggatgccttggaagccgtggaagagagagatgccatggagaaaattgaagactacgcagtgaaatccgggaggtttacttatcagaacgctgcagcccaaccagagacagggccaggaggatctcagtgcgtttgaagtcagcctgatctacttagtgaactcaggacagccaaggctatgtagagagccccgaagatgcaggctcttcagtattatgagaatgtacttaattttttcttgtagtagttagtgtatcatattattgtattatttatattattactgttaagtactatgttctcttattagaagttgaacacagaacctctgagaacacatatgctacaagtgttctaacacacctccagcatcccggattacctttgttcctgaacaaggcacaattggtagggtatgatagggcctgcctatcatcctaacactccggtgatggagccaggaagatcaagagttcgaggccagctggttcacataagatcccatataatgtgcaggatggctaaacttgctgagagctgactctgtggtctcctgtcccagattctagcgatattcattactaagacccttgtccagagacaaaagaccacctctgtaacagagggaagaataaaacagccctagggtggaaactccttgtgaacacagccactgctgtttactgttagactactgctcagcactacacagctgcacggcacctccctgtgccaggtgctagtgggcagcctactgagggtacatctaacttgaatctaacacacttgaagtgagtttgctggtttggacacagagggagcattaagtgctacctggggtgacccttgaggaccacgccccctgtaagcatttgaccattgtgagagtaaacactgaaactcaccattgtcctgcctcagcctttctagagctggaatcataggtatgctgcaccggatccagaaggagaaataactacctttagatactgtgatagggatttctagaaagctgccacatacagatttttgtcctgtgtgaattcctattgttttttgtttgtttttaattttttattatttattttcttcattcacatttcaaatgctatcccaaaagtcccctatgccctccccccccctgccccccgccctgctcccctacccactcactcactcccacttttttttttttttttgagacagggtttctctgtgtagccctggctgtcctggaactcactctgtagaccaggctggcctcgaactcagaaattcacctgcctctgcctcccaagtgctgggattaaaggtgtgtgccaccatgcccggccttttttttttttttttttaattaggtatttatttcatttaaatttccaatgctatcccaaaagtcccccacatgctcccccacccactccccacctgactttatatgcctcactcccacttcttggccctggtgttcccctgtactggggcatataacgtttgcaagaccaaggggcgtctctttccactgatggccgactaggccatcttctgatacatatgcagctagagacacgagctctgggggggtactggttagttcatattgttgttccacctatagggttgccgaccccttcagctccttgggtactttctctagctcctccactaggggccctgtgttctatccaatagatgactgtgagcatccacttctgtatttgccaggcactggcatagcctcatacgagacagctatatcagggtccagttttgttttgttttgttttaaactagcatgctggagaggtagctcagcggttaagagcagtggctgctcttccagaggtcctgagttccaattcccagcagctacatagtggctcacaaccttctctaatgggatctgatgtccttttctagtgtgtttgaagccagtggcagtgtaattacatacacaaaataaataaatctttttaaaaaagaaaaaaaaaaaaaaaaaaa

SEQ ID NO: 8 - DNA, 1180 bp, Homo sapiens
gctgtgggaacctctccacgcgcacgaactcagccaacgatttctgatagatttttgggagtttgaccagagatgcaaggggtgaaggagcgcttcctaccgttagggaactctggggacagagcgccccggccgcctgatggccgaggcagggtgcgacccaggacccaggacggcgtcgggaaccataccatggcccggatccccaagaccctaaagttcgtcgtcgtcatcgtcgcggtcctgctgccagtcctagcttactctgccaccactgcccggcaggaggaagttccccagcagacagtggccccacagcaacagaggcacagcttcaagggggaggagtgtccagcaggatctcatagatcagaacatactggagcctgtaacccgtgcacagagggtgtggattacaccaacgcttccaacaatgaaccttcttgcttcccatgtacagtttgtaaatcagatcaaaaacataaaagttcctgcaccatgaccagagacacagtgtgtcagtgtaaagaaggcaccttccggaatgaaaactccccagagatgtgccggaagtgtagcaggtgccctagtggggaagtccaagtcagtaattgtacgtcctgggatgatatccagtgtgttgaagaatttggtgccaatgccactgtggaaaccccagctgctgaagagacaatgaacaccagcccggggactcctgccccagctgctgaagagacaatgaacaccagcccagggactcctgccccagctgctgaagagacaatgaccaccagcccggggactcctgccccagctgctgaagagacaatgaccaccagcccggggactcctgccccagctgctgaagagacaatgaccaccagcccggggactcctgcctcttctcattacctctcatgcaccatcgtagggatcatagttctaattgtgcttctgattgtgtttgtttgaaagacttcactgtggaagaaattccttccttacctgaaaggttcaggtaggcgctggctgagggcggggggcgctggacactctctgccctgcctccctctgctgtgttcccacagacagaaacgcctgcccctgccccaaaaaaaaaaaaaaaaaaaaaaaaaaaaaaaaaaaaaaaaaaaaaaaaaaaaaaaaaaaaaaaaaaaaa

SEQ ID NO: 9 - DNA, 1726 bp, Homo sapiens
cgagaacctttgcacgcgcacaaactacggggacgatttctgattgatttttggcgctttcgatccaccctcctcccttctcatgggactttggggacaaagcgtcccgaccgcctcgagcgctcgagcagggcgctatccaggagccaggacagcgtcgggaaccagaccatggctcctggaccccaagatccttaagttcgtcgtcttcatcgtcgcggttctgctgccggtccgggttgactctgccaccatcccccggcaggacgaagttccccagcagacagtggccccacagcaacagaggcgcagcctcaaggaggaggagtgtccagcaggatctcatagatcagaatatactggagcctgtaacccgtgcacagagggtgtggattacaccattgcttccaacaatttgccttcttgcctgctatgtacagtttgtaaatcaggtcaaacaaataaaagttcctgtaccacgaccagagacaccgtgtgtcagtgtgaaaaaggaagcttccaggataaaaactcccctgagatgtgccggacgtgtagaacagggtgtcccagagggatggtcaaggtcagtaattgtacgccccggagtgacatcaagtgcaaaaatgaatcagctgccagttccactgggaaaaccccagcagcggaggagacagtgaccaccatcctggggatgcttgcctctccctatcactaccttatcatcatagtggttttagtcatcattttagctgtggttgtggttggcttttcatgtcggaagaaattcatttcttacctcaaaggcatctgctcaggtggtggaggaggtcccgaacgtgtgcacagagtccttttccggcggcgttcatgtccttcacgagttcctggggcggaggacaatgcccgcaacgagaccctgagtaacagatacttgcagcccacccaggtctctgagcaggaaatccaaggtcaggagctggcagagctaacaggtgtgactgtagagtcgccagaggagccacagcgtctgctggaacaggcagaagctgaagggtgtcagaggaggaggctgctggttccagtgaatgacgctgactccgctgacatcagcaccttgctggatgcctcggcaacactggaagaaggacatgcaaaggaaacaattcaggaccaactggtgggctccgaaaagctcttttatgaagaagatgaggcaggctctgctacgtcctgcctgtgaaagaatctcttcaggaaaccagagcttccctcatttaccttttctcctacaaagggaagcagcctggaagaaacagtccagtacttgacccatgccccaacaaactctactatccaatatggggcagcttaccaatggtcctagaactttgttaacgcacttggagtaatttttatgaaatactgcgtgtgataagcaaacgggagaaatttatatcagattcttggctgcatagttatacgattgtgtattaagggtcgttttaggccacatgcggtggctcatgcctgtaatcccagcactttgataggctgaggcaggtggattgcttgagctcgggagtttgagaccagcctcatcaacacagtgaaactccatctcaatttaaaaagaaaaaaagtggttttaggatgtcattctttgcagttcttcatcatgagacaagtctttttttctgcttcttatattgcaagctccatctct

SEQ ID NO: 10 - DNA, 22 bp, Artificial Sequence
  Description of Artificial Sequence Bcl-2 forward primer
acttcgcagagatgtccagtca

SEQ ID NO: 11 - DNA, 17 bp, Artificial Sequence
  Description of Artificial Sequence Bcl-2 reverse primer
tggcaaagcgtcccctc

SEQ ID NO: 12 - DNA, 20 bp, Artificial Sequence
  Description of Artificial Sequence Bcl-XL forward primer
tcgggatggagtaaactggg

SEQ ID NO: 13 - DNA, 16 bp, Artificial Sequence
  Description of Artificial Sequence Bcl-XL reverse primer
ccacgcacagtgcccc

SEQ ID NO: 14 - DNA, 18 bp, Artificial Sequence
  Description of Artificial Sequence FLIP forward primer
agcaaccgtggaggacca

SEQ ID NO: 15 - DNA, 23 bp, Artificial Sequence
  Description of Artificial Sequence FLIP reverse primer
ccatcagcaggaccctataatca

SEQ ID NO: 16 - DNA, 18 bp, Artificial Sequence
  Description of Artificial Sequence L32 forward primer
gaaactggcggaaaccca

SEQ ID NO: 17 - DNA, 20 bp, Artificial Sequence
  Description of Artificial Sequence L32 reverse primer
ggatctggcccttgaacctt

SEQ ID NO: 18 - DNA, 30 bp, Artificial Sequence
  Description of Artificial Sequence Primer
cgacgcgtccacatatggaagtttcaggtc

SEQ ID NO: 19 - DNA, 36 bp, Artificial Sequence
  Description of Artificial Sequence Primer
ggaagatcttgaaagcgaatgagttgtttttctggg